SEQUENCE LISTING (33 sequences)

SEQ ID NO: 1 - protein, 195 aa, Mus musculus
MGNHSGKRELSAEKASKDGEIHRGEAGKKRSVGKLSQTASEDSDVFGEADAIQNNGTSAEDTAVTDSKHTADPKNNWQGAHPADPGNRPHLIRLFSRDAPGREDNTFKDRPSESDELQTIQEDPTAASGGLDVMASQKRPSQRSKYLATASTMDHARHGFLPRHRDTGILDSIGRFFSGDRGAPKRGSGKVSSEP

SEQ ID NO: 2 - protein, 248 aa, Mus musculus
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAA

SEQ ID NO: 3 - protein, 247 aa, Mus musculus
MACLWSFSLPSCFLSLLLLLLLQLSCSYAGQFRVIGPGYPIRALVGDEAELPCRISPGKNATGMEVGWYRSPFSRVVHLYRNGKDQDAEQAPEYRGRTELLKETISEGKVTLRIQNVRFSDEGGYTCFFRDHSYQEEAAMELKVEDPFYWVNPGVLTLIALVPTILLQVSVGLVFLFLQHRLRGKLRAEVENLHRTFDPHFLRVPCWKITLFVIVPVLGPLVALIICYNWLHRRLAGQFLEELRNPL

SEQ ID NO: 4 - protein, 13 aa, Artificial Sequence
  Synthetic
HCLGKWLGHPDKF

SEQ ID NO: 5 - protein, 11 aa, Artificial Sequence
  Synthetic
NTWTCQSIAFP

SEQ ID NO: 6 - protein, 21 aa, Artificial Sequence
  Synthetic
MEVGWYRSPFSRVVHLYRNGK

SEQ ID NO: 7 - protein, 15 aa, Artificial Sequence
  Synthetic
DEGGYTCFFRDHSYQ

SEQ ID NO: 8 - DNA, 744 bp, Mus musculus
atggcctgtttgtggagcttctctttgcccagctgcttcctctcccttctcctcctccttctcctccagttgtcatgcagctatgcaggacaattcagagtgataggaccagggtatcccatccgggctttagttggggatgaagcagagctgccgtgccgcatctctcctgggaaaaatgccacgggcatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatcccttctattgggtcaaccccggtgtgctgactctcatcgcacttgtgcctacgatcctcctgcaggtctctgtaggccttgtattcctcttcctgcagcacagactgagaggaaaacttcgtgcagaagtagagaatctccatcggacttttgatcctcacttcctgagggtgccctgctggaagataacactgtttgttattgtgcctgttcttggacccctggttgccttgatcatctgctacaactggctgcaccgaagactggcaggacagtttcttgaagagctaagaaaccccctttga

SEQ ID NO: 9 - protein, 247 aa, Mus musculus
MACLWSFSWPSCFLSLLLLLLLQLSCSYAGQFRVIGPGYPIRALVGDEAELPCRISPGKNATGMEVGWYRSPFSRVVHLYRNGKDQDAEQAPEYRGRTELLKETISEGKVTLRIQNVRFSDEGGYTCFFRDHSYQEEAAMELKVEDPFYWVNPGVLTLIALVPTILLQVSVGLVFLFLQHRLRGKLRAEVENLHRTFDPHFLRVPCWKITLFVIVPVLGPLVALIICYNWLHRRLAGQFLEELRNPF

SEQ ID NO: 10 - DNA, 834 bp, Mus musculus
atgggcttgttagagtgttgtgctagatgtctggtaggggccccctttgcttccctggtggccactggattgtgtttctttggagtggcactgttctgtggatgtggacatgaagctctcactggtacagaaaagctaattgagacctatttctccaaaaactaccaggactatgagtatctcattaatgtgattcatgctttccagtatgtcatctatggaactgcctctttcttcttcctttatggggccctcctgctggctgagggcttctacaccaccggcgctgtcaggcagatctttggcgactacaagaccaccatctgcggcaagggcctgagcgcaacggtaacagggggccagaaggggaggggttccagaggccaacatcaagctcattctttggagcgggtgtgtcattgtttgggaaaatggctaggacatcccgacaagtttgtgggcatcacctatgccctgactgttgtatggctcctggtgtttgcctgctcggctgtacctgtgtacatttacttcaatacctggaccacctgtcagtctattgccttccctagcaagacctctgccagtataggcagtctctgcgctgatgccagaatgtatggtgttctcccatggaatgctttccctggcaaggtttgtggctccaaccttctgtccatctgcaaaacagctgagttccaaatgaccttccacctgtttattgctgcgtttgtgggtgctgcggccacactagtttccctgctcaccttcatgattgctgccacttacaacttcgccgtccttaaactcatgggccgaggcaccaagttctga

SEQ ID NO: 11 - protein, 277 aa, Unknown
  Homo sapiens or Mus musculus
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 12 - DNA, 588 bp, Mus musculus
atgggaaaccactctggaaagagagaattatctgctgagaaggccagtaaggatggagagattcaccgaggagaggctggaaagaagagaagcgtgggcaagctttctcagacggcctcagaggacagtgatgtgtttggggaggcagatgcgatccagaacaatgggacctcggctgaggacacggcggtgacagactccaagcacacagcagacccaaagaataactggcaaggcgcccacccagctgacccagggaaccgcccccacttgatccgcctcttttcccgagatgccccgggaagggaggacaacaccttcaaagacaggccctcagagtccgacgagcttcagaccatccaagaagaccccacagcagcttccggaggcctggatgtgatggcatcacagaagagaccctcacagcgatccaagtacctggccacagcaagtaccatggaccatgccaggcatggcttcctcccaaggcacagagacacgggcatccttgactccatcgggcgcttctttagcggtgacaggggtgcgcccaagcggggctctggcaaggtgagctccgagccgtag

SEQ ID NO: 13 - protein, 14 aa, Artificial Sequence
  Synthetic
NTWTTCQSIAFPSK

SEQ ID NO: 14 - DNA, 744 bp, Homo sapiens
atggcaagcttatcgagaccctctctgcccagctgcctctgctccttcctcctcctcctcctcctccaagtgtcttccagctatgcagggcagttcagagtgataggaccaagacaccctatccgggctctggtcggggatgaagtggaattgccatgtcgcatatctcctgggaagaacgctacaggcatggaggtggggtggtaccgcccccccttctctagggtggttcatctctacagaaatggcaaggaccaagatggagaccaggcacctgaatatcggggccggacagagctgctgaaagatgctattggtgagggaaaggtgactctcaggatccggaatgtaaggttctcagatgaaggaggtttcacctgcttcttccgagatcattcttaccaagaggaggcagcaatggaattgaaagtagaagatcctttctactgggtgagccctggagtgctggttctcctcgcggtgctgcctgtgctcctcctgcagatcactgttggcctcgtcttcctctgcctgcagtacagactgagaggaaaacttcgagcagagatagagaatctccaccggacttttgatccccactttctgagggtgccctgctggaagataaccctgtttgtaattgtgccggttcttggacccttggttgccttgatcatctgctacaactggctacatcgaagactagcagggcaattccttgaagagctacgaaatcccttctga

SEQ ID NO: 15 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLVFLCLQYRLRGKLRAEIENLHRTFDPHFLRVPCWKITLFVIVPVLGPLVALIICYNWLHRRLAGQFLEELRNPF

SEQ ID NO: 16 - DNA, 915 bp, Homo sapiens
atgggaaaccacgcaggcaaacgagaattaaatgccgagaaggccagtacgaatagtgaaactaacagaggagaatctgaaaaaaagagaaacctgggtgaactttcacggacaacctcagaggacaacgaagtgttcggagaggcagatgcgaaccagaacaatgggacctcctctcaggacacagcggtgactgactccaagcgcacagcggacccgaagaatgcctggcaggatgcccacccagctgacccagggagccgcccccacttgatccgcctcttttcccgagatgccccggggagggaggacaacaccttcaaagacaggccctctgagtccgacgagctccagaccatccaagaagacagtgcagccacctccgagagcctggatgtgatggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgcccaagcggggctctggcaaggactcacaccacccggcaagaactgctcactacggctccctgccccagaagtcacacggccggacccaagatgaaaaccccgtagtccacttcttcaagaacattgtgacgcctcgcacaccacccccgtcgcagggaaaggggagaggactgtccctgagcagatttagctggggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgctga

SEQ ID NO: 17 - protein, 304 aa, Homo sapiens
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 18 - DNA, 594 bp, Homo sapiens
atggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgcccaagcggggctctggcaaggtaccctggctaaagccgggccggagccctctgccctctcatgcccgcagccagcctgggctgtgcaacatgtacaaggactcacaccacccggcaagaactgctcactacggctccctgccccagaagtcacacggccggacccaagatgaaaaccccgtagtccacttcttcaagaacattgtgacgcctcgcacaccacccccgtcgcagggaaaggggagaggactgtccctgagcagatttagctggggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgctga

SEQ ID NO: 19 - protein, 197 aa, Homo sapiens
MASQKRPSQRHGSKYLATASTMDHARHGFLPRHRDTGILDSIGRFFGGDRGAPKRGSGKVPWLKPGRSPLPSHARSQPGLCNMYKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 20 - DNA, 561 bp, Homo sapiens
atggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgcccaagcggggctctggcaaggtaccctggctaaagccgggccggagccctctgccctctcatgcccgcagccagcctgggctgtgcaacatgtacaaggactcacaccacccggcaagaactgctcactacggctccctgccccagaagtcacacggccggacccaagatgaaaaccccgtagtccacttcttcaagaacattgtgacgcctcgcacaccacccccgtcgcagggaaagggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgctga

SEQ ID NO: 21 - protein, 186 aa, Homo sapiens
MASQKRPSQRHGSKYLATASTMDHARHGFLPRHRDTGILDSIGRFFGGDRGAPKRGSGKVPWLKPGRSPLPSHARSQPGLCNMYKDSHHPARTAHYGSLPQKSHGRTQDENPVVHFFKNIVTPRTPPPSQGKGAEGQRPGFGYGGRASDYKSAHKGFKGVDAQGTLSKIFKLGGRDSRSGSPMARR

SEQ ID NO: 22 - DNA, 516 bp, Homo sapiens
atggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgcccaagcggggctctggcaaggactcacaccacccggcaagaactgctcactacggctccctgccccagaagtcacacggccggacccaagatgaaaaccccgtagtccacttcttcaagaacattgtgacgcctcgcacaccacccccgtcgcagggaaaggggagaggactgtccctgagcagatttagctggggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgctga

SEQ ID NO: 23 - protein, 171 aa, Homo sapiens
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 24 - DNA, 483 bp, Homo sapiens
atggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgcccaagcggggctctggcaaggactcacaccacccggcaagaactgctcactacggctccctgccccagaagtcacacggccggacccaagatgaaaaccccgtagtccacttcttcaagaacattgtgacgcctcgcacaccacccccgtcgcagggaaagggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgctga

SEQ ID NO: 25 - protein, 160 aa, Homo sapiens
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGAEGQRPGFGYGGRASDYKSAHKGFKGVDAQGTLSKIFKLGGRDSRSGSPMARR

SEQ ID NO: 26 - DNA, 594 bp, Homo sapiens
atgggaaaccacgcaggcaaacgagaattaaatgccgagaaggccagtacgaatagtgaaactaacagaggagaatctgaaaaaaagagaaacctgggtgaactttcacggacaacctcagaggacaacgaagtgttcggagaggcagatgcgaaccagaacaatgggacctcctctcaggacacagcggtgactgactccaagcgcacagcggacccgaagaatgcctggcaggatgcccacccagctgacccagggagccgcccccacttgatccgcctcttttcccgagatgccccggggagggaggacaacaccttcaaagacaggccctctgagtccgacgagctccagaccatccaagaagacagtgcagccacctccgagagcctggatgtgatggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgcccaagcggggctctggcaaggtgagctctgaggagtag

SEQ ID NO: 27 - protein, 197 aa, Homo sapiens
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKVSSEE

SEQ ID NO: 28 - DNA, 834 bp, Homo sapiens
atgggcttgttagagtgctgtgcaagatgtctggtaggggccccctttgcttccctggtggccactggattgtgtttctttggggtggcactgttctgtggctgtggacatgaagccctcactggcacagaaaagctaattgagacctatttctccaaaaactaccaagactatgagtatctcatcaatgtgatccatgccttccagtatgtcatctatggaactgcctctttcttcttcctttatggggccctcctgctggctgagggcttctacaccaccggcgcagtcaggcagatctttggcgactacaagaccaccatctgcggcaagggcctgagcgcaacggtaacagggggccagaaggggaggggttccagaggccaacatcaagctcattctttggagcgggtgtgtcattgtttgggaaaatggctaggacatcccgacaagtttgtgggcatcacctatgccctgaccgttgtgtggctcctggtgtttgcctgctctgctgtgcctgtgtacatttacttcaacacctggaccacctgccagtctattgccttccccagcaagacctctgccagtataggcagtctctgtgctgatgccagaatgtatggtgttctcccatggaatgctttccctggcaaggtttgtggctccaaccttctgtccatctgcaaaacagctgagttccaaatgaccttccacctgtttattgctgcatttgtgggggctgcagctacactggtttccctgctcaccttcatgattgctgccacttacaactttgccgtccttaaactcatgggccgaggcaccaagttctga

SEQ ID NO: 29 - protein, 222 aa, Homo sapiens
MDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 30 - DNA, 729 bp, Homo sapiens
atgggcttgttagagtgctgtgcaagatgtctggtaggggccccctttgcttccctggtggccactggattgtgtttctttggggtggcactgttctgtggctgtggacatgaagccctcactggcacagaaaagctaattgagacctatttctccaaaaactaccaagactatgagtatctcatcaatgtgatccatgccttccagtatgtcatctatggaactgcctctttcttcttcctttatggggccctcctgctggctgagggcttctacaccaccggcgcagtcaggcagatctttggcgactacaagaccaccatctgcggcaagggcctgagcgcaacgtttgtgggcatcacctatgccctgaccgttgtgtggctcctggtgtttgcctgctctgctgtgcctgtgtacatttacttcaacacctggaccacctgccagtctattgccttccccagcaagacctctgccagtataggcagtctctgtgctgatgccagaatgtatggtgttctcccatggaatgctttccctggcaaggtttgtggctccaaccttctgtccatctgcaaaacagctgagttccaaatgaccttccacctgtttattgctgcatttgtgggggctgcagctacactggtttccctgctcaccttcatgattgctgccacttacaactttgccgtccttaaactcatgggccgaggcaccaagttctga

SEQ ID NO: 31 - protein, 242 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 32 - DNA, 669 bp, Homo sapiens
atggactatgagtatctcatcaatgtgatccatgccttccagtatgtcatctatggaactgcctctttcttcttcctttatggggccctcctgctggctgagggcttctacaccaccggcgcagtcaggcagatctttggcgactacaagaccaccatctgcggcaagggcctgagcgcaacggtaacagggggccagaaggggaggggttccagaggccaacatcaagctcattctttggagcgggtgtgtcattgtttgggaaaatggctaggacatcccgacaagtttgtgggcatcacctatgccctgaccgttgtgtggctcctggtgtttgcctgctctgctgtgcctgtgtacatttacttcaacacctggaccacctgccagtctattgccttccccagcaagacctctgccagtataggcagtctctgtgctgatgccagaatgtatggtgttctcccatggaatgctttccctggcaaggtttgtggctccaaccttctgtccatctgcaaaacagctgagttccaaatgaccttccacctgtttattgctgcatttgtgggggctgcagctacactggtttccctgctcaccttcatgattgctgccacttacaactttgccgtccttaaactcatgggccgaggcaccaagttctga

SEQ ID NO: 33 - protein, 9 aa, Artificial Sequence
  Synthetic
GRVRVNSAY

(19) United States
(12) Patent Application Publication
(10) Pub. No.: US 2023/0263909 A1
(43) Pub. Date: Aug. 24, 2023

(54) ENHANCED EFFECTS OF GENE-IMMUNOTHERAPY AND IMMUNOSUPPRESSANTS IN MULTIPLE SCLEROSIS

(71) Applicant: University of Florida Research Foundation, Incorporated, Gainesville, FL (US)

(72) Inventor: Brad E. Hoffman, Gainesville, FL (US)

(73) Assignee: University of Florida Research Foundation, Incorporated, Gainesville, FL (US)

(21) Appl. No.: 17/919,052

(22) PCT Filed: Apr. 13, 2021
(86) PCT No.: PCT/US2021/027167

§ 371 Date: Oct. 14, 2022

Related U.S. Application Data
(60) Provisional application No. 63/010,028, filed on Apr. 14, 2020.

(51) Int. Cl. A61K 48/00; A61K 31/573; C12N 15/86; A61K 31/436; A61K 31/137; A61K 9/00; A61P 25/00; A61P 37/06

(52) U.S. Cl. CPC A61K 48/0058; A61K 9/0019; A61K 9/0053; A61K 31/137; A61K 31/436; A61K 31/573; A61P 25/00; A61P 37/06; C12N 15/86; C12N 2750/14143

(57) ABSTRACT

Disclosed are methods of administering AAV viral-based vector compositions useful in delivering a variety of nucleic acid segments and compositions comprising an agent, such as an immunosuppressive agent. Methods and compositions comprising a combination therapy are provided. The disclosed AAV vector compositions include those encoding therapeutic polypeptides to selected mammalian host cells for use in therapeutic autoimmune modalities, including, for example, the in vivo induction of immunological tolerance via a liver-directed AAV -based gene therapeutic regimen for treating and/or ameliorating autoimmune disorders such as multiple sclerosis. The compositions comprising an agent may comprise a sphingosine analog, a glucococorticoid, an mTOR inhibitor, and/or a targeted biologic.

### RELATED APPLICATIONS

This application claims the benefit of the filing date of U.S. Provisional Application No. 63/010,028, filed Apr. 14, 2020, the entire contents of which are incorporated by reference.

### FIELD OF THE INVENTION

The present invention relates generally to the fields of molecular biology and virology, and in particular, to the development of gene therapy vectors and methods for treatment of autoimmune diseases, such as multiple sclerosis (MS).

### BACKGROUND OF THE INVENTION

Multiple Sclerosis (MS). MS is a multifocal demyelinating disease with progressive neurodegeneration caused by an autoimmune response to self-antigens in a genetically susceptible individual. Depending on where in the CNS the damage occurs, symptoms may include problems with muscle control, balance, vision, or speech. It is estimated that MS affects 250,000 to 350,000 people in the US alone. MS is an autoimmune disease that develops (in part) from a failure of central and peripheral tolerance mechanisms (particularly regulatory T cells, i.e., Tregs) to maintain self-tolerance and control potentially pathogenic autoreactive lymphocytes.^{2,3} It is characterized by chronic lymphocyte infiltration and inflammation of the CNS, resulting in demyelination.

Gene Therapy. Major advances in the field of gene therapy have been achieved by using viruses to deliver therapeutic genetic material. The adeno-associated virus (AAV) has attracted considerable attention as a highly effective viral vector for gene therapy due to its low immunogenicity and ability to effectively transduce non-dividing cells. AAV has been shown to infect a variety of cell and tissue types, and significant progress has been made over the last decade to adapt this viral system for use in human gene therapy.

Recombinant adeno-associated virus (rAAV) vectors have been used successfully for in vivo gene transfer in numerous pre-clinical animal models of human disease, and have been used successfully for long-term expression of a wide variety of therapeutic genes (Daya and Berns, 2008; Niemeyer et al., 2009; Owen et al., 2002; Keen-Rhinehart et al., 2005; Scallan et al., 2003; Song et al., 2004). rAAV vectors have also generated long-term clinical benefit in humans when targeted to immune-privileged sites, e.g., ocular delivery for Leber’s congenital amaurosis (Bainbridge et al., 2008; Maguire et al., 2008; Cideciyan et al., 2008). A major advantage of this vector is its comparatively low immune profile, eliciting only limited inflammatory responses and, in some cases, even directing immune tolerance to transgene products (LoDuca et al., 2009). Nonetheless, the therapeutic efficiency, when targeted to non-immune privileged organs, has been limited in humans due to antibody and CD8⁺ T cell responses against the viral capsid. Adaptive responses to the transgene product have also been reported in animal models (Manno et al., 2006; Mingozzi et al., 2007; Muruve et al., 2008; Vandenberghe and Wilson, 2007; Mingozzi and High, 2007).

In its normal “wild type” form, AAV DNA is packaged into the viral capsid as a single-stranded molecule about 4600 nucleotides (nt) in length. Following infection of the cell by the virus, the molecular machinery of the cell converts the single-stranded DNA into a double-stranded form. Only this double-stranded DNA form can be transcribed by cellular enzymes into RNA, which is then translated into polypeptides by additional cellular pathways.

### SUMMARY OF THE INVENTION

The present disclosure provides viral vector-based gene therapy methods for treating and/or ameliorating one or more of symptoms of autoimmune diseases in human subjects. In particular, the present disclosure provides recombinant AAV (rAAV)-based gene therapy methods for tolerization of immune cells that are implicated in autoimmune diseases, such as multiple sclerosis (MS). The development of such vectors, and compositions comprising them, provides a major advancement in medicine, and particularly in the development of a gene therapy-based treatment modality for MS.

The inventor has discovered that the use of specific immunosuppressive agents (or immunsuppressants) with neuropeptide-encoding rAAV vectors in a combination therapy provides for a synergistic effect on transduction and tolerization. Accordingly, the disclosed methods and compositions overcomes limitations of the prior art by providing compositions of AAV nucleic acid vectors and an immunosuppressive agent, and methods of treatment comprising administration of these compounds, that are capable of inducing a robust antigen-specific immune tolerance.. The disclosed methods of treatment abrogate the need for identifying HLA-MHC-specific epitopes required for inducing antigen-specific Tregs. In some embodiments, these methods permits each patient undergoing treatment to generate his/her own unique antigen-specific Tregs, which makes the treatment more universally applicable and more clinically feasible than existing technologies.

2 million people worldwide are living with MS. Diagnosis generally occurs at ages of 20 to 40, but documented cases of MS in children as young as two have been reported. Schilder’s disease is a rare progressive demyelinating disorder that usually begins in childhood. While there is currently no cure for MS, there are various MS treatment options which have shown a decrease in the severity and frequency of relapses and a delay in disease progression in numerous studies. Therapies with predominantly immunomodulating properties include e.g., Beta-interferons and Glatiramer acetate. Therapies with predominantly immunosuppressive properties include e.g., Natalizumab, Fingolimod, and Mitoxantrone. The development of protocols that stimulates Treg numbers and/or their function has become a significant focus in treating autoimmune diseases. In fact, many of the beneficial effects of currently FDA approved immune-modulators used in the treatment of MS are associated with restored Treg homeostasis.^{2,4,5}

AAV gene therapy has been proven to be a powerful new tool for the treatment of a broad spectrum of diseases, including restoration of vision in patients with Leber congenital amaurosis by retinal gene transfer, and treatment of hemophilia B by hepatic gene therapy.^{6,7} According to aspects of the disclosure, it has been demonstrated that hepatic gene therapy transfer with AAV vectors can reliably induce a robust, antigen-specific immune tolerance to a variety of proteins in experimental animals, even when the antigen is subsequently expressed in a highly immunogenic manner in other organs. Together, these results demonstrate that liver-directed gene therapy can abrogate potentially cytotoxic CD8⁺ T cell responses.^{1,8-13} Importantly, it has also been shown that this protocol can even eliminate pre-existing antibodies.¹ This finding is quite significant since there is an increasing body of evidence that B cells and auto-antibodies may play a pathogenic role in demyelinating disease.^{14,15} Others have shown that transgenic mice or transient transfection by plasmid or adenovirus vectors expressing myelin basic protein could prevent the onset of Experimental autoimmune encephalomyelitis (EAE) disease in mice.^{16,17} Suppression was dependent on hepatic gene expression and was mediated by induction of antigen-specific Tregs. In contrast, aspects of the disclosure relate to treatment of certain autoimmune conditions, e.g., MS, utilizing AAV delivery of nucleic acids encoding one or more host proteins to the liver.

Hepatocyte-restricted transgene expression from an optimized AAV vector can reliably induce immune tolerance to various therapeutic proteins (e.g., mediated by antigen-specific CD4⁺CD25⁺FoxP3⁺ Tregs). The process suppresses antibody formation and cytotoxic CD8⁺ T cell response against the transgene product. Hepatic transgene expression is maintained even when the antigen is subsequently expressed in a highly immunogenic manner in other organs. The process efficiently and rapidly reverses pre-existing high antibody titers, and provids long-term correction of haemostasis in a murine hemophilia B model. Importantly, the method does not require protein to be secreted to be functional.

Accordingly, in some aspects, the disclosure provides methods and compositions for treating subjects having, or suspected of having, an autoimmune disease (e.g., multiple sclerosis) with a combination of an rAAV that encodes one or more neuropeptides (e.g., tolerizing neuropeptides) and one or more immunosuppressive agents (e.g., an mTOR inhibitor, a sphingosine analog, a targeted biologic, or a glucocorticoid). In various embodiments, the disclosure provides for compositions comprising an mTOR inhibitor immunosuppressive agent, e.g., rapamycin. In various embodiments, the disclosure provides for compositions comprising a sphingosine analog immunosuppressive agent, e.g., an oral sphingosine analog agent such as fingolimod. In various embodiments, the disclosure provides for compositions comprising a glucocorticoid immunosuppressive agent, e.g., prednisolone.

In some embodiments of the compositions and methods provided herein, any of the disclosed rAAV particles are co-administered with rapamycin. In some embodiments of the compositions and methods provided herein, any of the disclosed rAAV particles are co-administered with fingolimod.

In some embodiments of the compositions and methods provided herein, any of the disclosed rAAV particles are co-administered with prednisolone. In some embodiments of the compositions and methods provided herein, any of the disclosed rAAV particles are co-administered with natalizumab.

Advantageously, the disclosed compositions of i) rAAV nucleic acid vectors, and infectious virions and viral particles comprising them, and ii) immunosuppressive agents disclosed herein may have an improved efficiency in transducing one or more mammalian liver cells to provide persistent expression of one or more genes of interest. The compositions of rAAV nucleic acid vectors and immunosuppressive agents provided herein may transduce mammalian cells with sufficient transduction efficiency to suppress the immune response associated with MS in patients, and thus abrogate CNS inflammation, and immune-mediated damage that occurs in MS patients. Unlike current therapies, this gene-therapy based approach represents a persistent, long-term treatment that reduces the clinical disability experienced by MS patients.

The present disclosure further provides compositions and formulations that include one or more of the proteins, nucleic acid segments, viral vectors, host cells, or viral particles of the present invention together with one or more pharmaceutically-acceptable buffers, diluents, or excipients. Such compositions may be included in one or more diagnostic or therapeutic kits, for diagnosing, preventing, treating or ameliorating one or more symptoms of a mammalian inflammatory disease, such as autoimmune disease, and in particular, for delivery of a therapeutic agent for the treatment of MS in a human.

The present disclosure further includes a method for providing a mammal in need thereof with a diagnostically- or therapeutically-effective amount of a selected therapeutic agent, the method comprising administering to a cell, tissue or organ of a mammal in need thereof, an amount of one or more of the disclosed rAAV nucleic acid vectors; and for a time effective to provide the mammal with a diagnostically- or a therapeutically-effective amount of the selected therapeutic agent.

The present disclosure further provides a method for diagnosing, preventing, treating, or ameliorating at least one or more symptoms of a disease, a disorder, a dysfunction, an injury, an abnormal condition, or trauma in a mammal (e.g., a human). In an overall and general sense, the method includes at least the step of administering to a mammal in need thereof one or more of the disclosed rAAV nucleic acid vectors, in an amount and for a time sufficient to diagnose, prevent, treat or ameliorate the one or more symptoms of the disease, disorder, dysfunction, injury, abnormal condition, or trauma in the mammal. The disclosed methods may be particularly useful for treating human subjects in need thereof, for example human MS patients.

The present disclosure also provides a method of transducing a population of mammalian cells. In an overall and general sense, the method includes at least the step of introducing into one or more cells of the population, a composition that comprises an effective amount of one or more of the rAAV nucleic acid vectors disclosed herein.

In some embodiments, the present disclosure also provides isolated nucleic acid segments that encode one or more of the rAAV vector-based gene therapy constructs as described herein, and provides recombinant vectors, virus particles, infectious virions, and isolated host cells that comprise one or more of the rAAV nucleic acid vectors described herein.

Additionally, the present invention provides compositions, as well as therapeutic and/or diagnostic kits that include one or more of the disclosed AAV nucleic acid vector or AAV particle compositions, formulated with one or more additional ingredients, or prepared with one or more instructions for their use.

In some aspect, the present disclosure provides compositions comprising recombinant adeno-associated viral (AAV) nucleic acid vectors, virions, and viral particles, and pharmaceutical formulations thereof, useful in methods for delivering genetic material encoding one or more beneficial or therapeutic product(s) to mammalian cells and tissues. In some embodiments, the compositions and methods of the present disclosure provide a significant advancement in the art through their use in the treatment, prevention, and/or amelioration of symptoms of one or more mammalian inflammatory diseases, including autoimmune diseases such as MS and the like.

Some embodiments contemplate a method of treating a mammal in need thereof comprising administering to the mammal a therapeutically-effective amount of: (a) a first composition comprising a recombinant adeno-associated viral (rAAV) vector comprising a polynucleotide that encodes a mammalian myelin basic protein (MBP), a proteolipid protein (PLP), or a myelin oligodendrocyte glycoprotein (MOG) operably linked to a promoter that is capable of expressing the nucleic acid segment in one or more cells of a mammalian liver; and (b) a second composition comprising an agent comprising a sphingosine analog. In some embodiments, the nucleic acid segment encodes a first therapeutic molecule that comprises one of a myelin basic protein (MBP), a myelin oligodendrocyte glycoprotein (MOG), and a proteolipid protein (PLP).

In some aspects, the second composition comprises an mTOR inhibitor. In other aspects, the second composition comprises a monoclonal antibody.

In some embodiments, the nucleic acid segment encodes a myelin basic protein (MBP), a proteolipid protein (PLP), or a myelin oligodendrocyte glycoprotein (MOG) that comprises an amino acid sequence that is at least 90% identical to the sequence as set forth in SEQ ID NO:1, SEQ ID NO:2, or SEQ ID NO:3. In some embodiments, the nucleic acid segment encodes a myelin basic protein (MBP), a proteolipid protein (PLP), or a myelin oligodendrocyte glycoprotein (MOG) that comprises an amino acid sequence as set forth in SEQ ID NO:1, SEQ ID NO:2, or SEQ ID NO:3.

In some embodiments, the nucleic acid segment encodes a myelin basic protein (MBP), proteolipid protein (PLP), or myelin oligodendrocyte glycoprotein (MOG) that comprises an amino acid sequence that is at least 90% identical to the sequence as set forth in SEQ ID NO:17, SEQ ID NO:11, or SEQ ID NO:15. In some embodiments, the nucleic acid segment encodes a myelin basic protein (MBP), proteolipid protein (PLP), or myelin oligodendrocyte glycoprotein (MOG) that comprises an amino acid sequence as set forth in SEQ ID NO:17, SEQ ID NO:11, or SEQ ID NO:15. In some embodiments, the MBP, PLP or MOG are of human origin.

In some embodiments, the promoter is a hepatocyte-specific promoter. In some embodiments, the hepatocyte-specific promoter comprises an albumin promoter, a human α₁-antitrypsin promoter, a transthyretin (TTR) promoter, a hepatic combinatorial bundle (HCB) promoter, or an apolipoprotein E (apoE) promoter. In some embodiments, the hepatocyte-specific promoter comprises a hepatic combinatorial bundle (HCB) promoter. In some embodiments, the hepatocyte-specific promoter comprises a human apolipoprotein E (hapoE) promoter.

The present invention also concerns rAAV nucleic acid vectors, wherein the nucleic acid segment further comprises a promoter, an enhancer, a post-transcriptional regulatory sequence, a polyadenylation signal, or any combination thereof, operably linked to the nucleic acid segment that encodes the selected polynucleotide of interest. Thus, in some embodiments, the polynucleotide further comprises an enhancer, a post-transcriptional regulatory sequence, a polyadenylation signal, or any combination thereof, operably linked to the nucleic acid segment. In some embodiments, the polynucleotide comprises AAV2 inverted terminal repeat sequences (ITRs).

In some embodiments, the polynucleotide comprises a second nucleic acid segment (or sequence) encoding a second therapeutic molecule. In some embodiments, the second therapeutic molecule is MBP or PLP if the first therapeutic molecule is MOG. In some embodiments, the second therapeutic molecule is MBP or MOG if the first therapeutic molecule is PLP. In some embodiments, the second therapeutic molecule is PLP or MOG if the first therapeutic molecule is MBP.

In some embodiments, the second nucleotide sequence or the second autoimmune disease therapeutic molecule of interest is not necessary for the full therapeutic function of the rAAV.

The present disclosure further provides compositions and formulations that include one or more of the proteins, nucleic acid segments, viral vectors, host cells, or viral particles of the present invention, together with one or more pharmaceutically-acceptable buffers, diluents, or excipients. Such compositions may be included in one or more diagnostic or therapeutic kits for diagnosing, preventing, treating or ameliorating one or more symptoms of a mammalian inflammatory disease, such as autoimmune disease, and in particular, for delivery of a therapeutic agent for the treatment of MS in a human.

The present disclosure further includes a method for providing a mammal (e.g., a human) in need thereof with a diagnostically- or therapeutically-effective amount of a selected therapeutic agent, the method comprising administering to a cell, tissue or organ of a mammal in need thereof an amount of one or more of the disclosed rAAV nucleic acid vectors. In some embodiments, administration is continued for a time which is effective to provide the mammal with a diagnostically- or a therapeutically-effective amount of the selected therapeutic agent.

In some embodiments, an rAAV vector of the present disclosure is used to treat an autoimmune disease. In some embodiments, the autoimmune disease is selected from multiple sclerosis, disseminated sclerosis, encephalomyelitis disseminata, optic neuritis, celiac disease, diabetes, Graves’ disease, Hashimoto’s disease, hyperthyroidism, or an allergic disease. In some embodiments, the autoimmune disease is multiple sclerosis.

The present disclosure further provides a method for diagnosing, preventing, treating, and/or ameliorating at least one symptom of a disease, a disorder, a dysfunction, an injury, an abnormal condition, and/or trauma in a mammal (e.g., a human). In an overall and general sense, the method includes at least the step of administering to the mammal in need thereof one or more of the disclosed rAAV nucleic acid vectors, in an amount and for a time sufficient to diagnose, prevent, treat or ameliorate the one or more symptoms of the disease, disorder, dysfunction, injury, abnormal condition, or trauma in the mammal.

### BRIEF DESCRIPTION OF THE DRAWINGS

The following drawings form part of the present specification and are included to demonstrate certain aspects of the present invention. The present disclosure may be better understood by reference to the following description taken in conjunction with the accompanying drawings, in which like reference numerals identify like elements, and in which:

FIG. 1 describes aspects of an experimental autoimmune encephalomyelitis (EAE) murine model employed in the present study as an animal model for MS.

FIG. 2A and FIG. 2B show a mouse model and mean clinical score criteria for the EAE study.

FIG. 3 shows a comparison of exemplary methods of the present invention as contrasted with the cell-based delivery methods of the prior art.

FIG. 4A and FIG. 4B show the AAV8 expression of MOG. FIG. 4A shows the Western blot analysis from protein extracted from liver, while FIG. 4B shows the analysis of transcriptional levels using real-time RT-PCR.

FIG. 5A and FIG. 5B show the mean clinical score of EAE mice. In FIG. 5A, five female mice were injected subcutaneously with antigen in complete Freund’s adjuvant (Ag/CFA) emulsion. Mean clinical score (± standard error of measurement (SEM)) was recorded starting at day 12. In FIG. 5B, five female C57BL/6 mice were injected subcutaneously with MOG/CFA emulsion. Mean clinical score (±SEM) was recorded.

FIG. 6A, FIG. 6B, and FIG. 6C show AAV8-MOG prevented development of EAE in C57BL/6 mice. C57BL/6 mice (n=5) were injected with AAV8-MOG or control. EAE was induced 2 weeks later. FIG. 6A: Mean clinical score, FIG. 6B: anti-MOG IgG1, FIG. 6C: anti-MOG IgG2c.

FIG. 7A, FIG. 7B, and FIG. 7C show that AAV8-vectored gene therapy prevents the onset of EAE in the animal model of MS.

FIG. 8 shows that AAV8-MOG ameliorated the disease in the animal model of MS.

FIG. 9 shows a PLP-induced EAE naive control group to demonstrate disease progression.

FIG. 10 shows effective suppression of pre-existing disease using the AAV8-vectored MOG treatment.

FIG. 11 shows hepatic transgene expression of MOG. Western blot analysis from protein extracted from liver of MOG induced EAE mice injected with AAV8-MOG.

FIG. 12 shows Luxol Fast Blue (LFB) staining of spinal cords from mice that received AAV8-GFP and had EAE induced (left) or not (right).

FIG. 13A, FIG. 13B, and FIG. 13C show mean clinical score (MCS) in EAE-induced C57BL/6 mice that received AAV8-MOG or control vector after the mice reached a specific MCS. FIG. 13A shows MCS in EAE-induced C57BL/6 mice that received AAV8-MOG or control vector after the mice reached a MCS of about 0.3. FIG. 13B shows mean clinical score (MCS) in EAE-induced C57BL/6 mice that received AAV8-MOG or control vector after the mice reached a MCS of about 0.8. FIG. 13C shows mean clinical score (MCS) in EAE-induced C57BL/6 mice that received AAV8-MOG or control vector after the mice reached a MCS of about 1.3. Bar graphs show statistical significance between final scores and peak-to-final scores throughout.

FIG. 14A and FIG. 14B show serial sections of spinal cord from an EAE induced female mouse ~35 days after receiving control vector (MCS=4.0). FIG. 14A is a hematoxylin and eosin (H&E) stain showing areas of high inflammatory infiltration. FIG. 14B is a Luxol fast blue stain showing areas of demyelination. Circled areas highlight the co-localization of inflammation and loss of myelin.

FIG. 15A and FIG. 15B show serial sections of spinal cord from an EAE induced female mouse ~35 days after receiving AAV-MOG vector (MCS =1.25). FIG. 15A is a hematoxylin and eosin stain showing diminished infiltration. FIG. 15B is a Luxol fast blue stain which shows that the section has less areas of demyelination as a result of the suppression of the inflammation.

FIG. 16A and FIG. 16B show that Tregs isolated from spleens of AAV-MOG treated mice are functionally suppressive.

FIG. 17A, FIG. 17B, FIG. 17C and FIG. 17D show that AAV-MOG vector induces antigen specific Tregs. Splenocytes from mice injected with AAV-MOG vector 8 weeks prior showed an increase in frequencies of I-Ab MOG₃₅₋₅₅ Tetramer positive CD4+ (FIG. 17A) and Treg+ (FIG. 17C) compared to control tetramer positive CD+ (FIG. 17B) and Treg+ (FIG. 17D).

FIG. 18 shows that AAV8-PLP reduces clinical severity in mice with PLP-induced relapsing-remitting EAE.

FIG. 19A and FIG. 19B show testing of a MBP vector. FIG. 19A shows a Western blot analysis from protein extracted from liver of mice injected with AAV-MBP. FIG. 19B shows analysis of transcriptional levels of RNA obtained from the liver of mice treated with AAV-MBP or control by real-time RT-PCR.

FIG. 20A, FIG. 20B, FIG. 20C, FIG. 20D, FIG. 20E, and FIG. 20F show that functional Ag-specific Tregs are induced following AAV8.MOG injection. C56B⅙ mice were injected with 10¹¹ vg of AAV8-MOG via tail vein. FIG. 20A shows Western blot analysis from liver lysates obtained from mice injected with AAV8.MOG 200 days after EAE or control (AAV8.GFP; lane C). Lane M is a molecular size marker in kDa. FIG. 20B shows real-time qPCR analysis to estimate the transgene copy number from liver lysates (±SD) (n = 4). FIG. 20C shows representative flow cytometry analysis of freshly isolated splenocytes from FOXP3^{gfp+} reporter mice tolerized with AAV8.MOG vector that were stained ex vivo with MOG/I-A^{b} or h.CLIP/I-A^{b} (control) tetramers. FIG. 20D shows statistical comparison of I-A^{b} MOG and I-A^{b} h.CLIP (control) tetramer populations of CD4⁺CD25⁺FOXP3⁺ Tregs from mice that received AAV.MOG vector (n = 8; U = 0; p = 0.0002, two-tailed Mann-Whitney U test). FIG. 20E and FIG. 20F show an in vitro Treg suppression assay. FIG. 20E shows FOXP3^{gfp+} Tregs isolated from mice after being tolerized with AAV.MOG were co-cultured at indicated concentrations with MOG-specific 2D2 T cells in the presence of 1 µg/µL MOG₃₅₋₅₅ peptide. FIG. 20F shows the mean % suppression of Tregs (n = 3; 1:160 versus 1:10: t = 9.753, df = 3.967, p = 0.0006; 1:40 versus 1:10: t = 4.565, df = 2.705, p = 0.0246, unpaired t test with Welch’s correction; experiment was repeated twice). Data are presented with mean values as indicated; error bars show ± SD. *p < 0.05; ***p < 0.001.

FIG. 21A, FIG. 21B, FIG. 21C, FIG. 21D, FIG. 21E and FIG. 21F show that the prophylactic administration of AAV8.MOG protects mice from EAE. C57BL/6 mice (9 weeks old) were intravenously injected with 10¹¹ vg/mouse via the tail vein with either AAV8.MOG or AAV8.GFP/control vector (day -14). Two weeks later (day 0), EAE was induced with MOG₃₅₋₅₅/CFA. FIG. 21A shows the experimental scheme and initial timeline in days. FIG. 21B shows MCS (± SEM) of AAV8.MOG-treated mice and control mice (n = 5 per group; ****p < 0.0001, two-tailed t test, Mann-Whitney test). Experiments were reproduced at least twice. FIG. 21C shows anti-MOG₃₅₋₅₅ titers measured via ELISA (mean ± SEM) (n = 3 per group). FIG. 21D shows IgG2c antibody titers measured via ELISA (mean ± SEM) (n = 3 per group). FIG. 21E shows the frequency of CD4⁺CD25⁺FOXP3⁺ Tregs (mean ± SD) present in blood at 5 weeks after vector administration (n = 6 group; U = 4; p = 0.0260, two-tailed Mann-Whitney U test). FIG. 21F shows plasma alanine aminotransferase (ALT) enzyme levels (IU/L) from age-matched naive control mice and vector-treated mice at 105 days post-injection (n = 10 per group).

FIG. 22A, FIG. 22B, FIG. 22C and FIG. 22D show that AAV8.MOG-induced immune tolerance is robust. Age-matched C57BL/6 mice (9-10 weeks old) were intravenously injected with 10¹¹ vg/mouse via the tail vein with either AAV8.MOG or PBS/control vector. EAE was induced with MOG₃₅₋₅₅/CFA 200 days later and re-challenged after 84 more days. FIG. 22A shows the experimental scheme and initial timeline in days. FIG. 22B shows MCS (±SEM) of AAV8.MOG-treated mice and control mice (n = 9-10 per group; p < 0.0001, two-tailed t test, Mann-Whitney test). Right panel: blow-out-treated mice showing only 2 of 10 developed relapsing-remitting EAE. FIG. 22C shows the survival curve of mice (p > 0.0001, log rank [Mantel-Cox] test). FIG. 22D shows plasma ALT levels (IU/L) from age-matched naive control mice and vector treated at various time points. Dashed line is time of re-challenge.

FIG. 23A, FIG. 23B, FIG. 23C and FIG. 23D show that AAV8.MOG induces clinical and pathological remission of EAE. EAE was induced in 9-week-old female C57BL/6 mice using MOG₃₅₋₅₅ in CFA. MCS (mean ± SEM) was recorded, and as mice developed increasing neurological symptoms, was recorded as increasing MCS. Mice were intravenously injected with either 10¹¹ vector genomes (vg) AAV8.MOG or control via the tail vein in an alternating fashion. FIG. 23A shows MCS 0.3, loss of tail tonality (n = 5; final control versus final AAV8.MOG: q = 0.9342, degrees of freedom (D.F.) 12, p < 0.0001; peak AAV8.MOG versus final AAV8.MOG: q = 10.74, D.F. 12, p < 0.0001). FIG. 23B shows MCS 0.8, tail paralysis (n = 9-10; final control versus final AAV8.MOG: q = 9.042, D.F. 30, p < 0.0001; peak AAV8.MOG versus final AAV8.MOG: t = 8.627, D.F. 30, p < 0.0001). FIG. 23C shows MCS 1.3, tail paralysis with hind-leg paresis (n = 5; final control versus final AAV8.MOG: q = 4.358, D.F. 12, p = 0.0412; peak AAV8.MOG versus final AAV8.MOG: q = 6.9, D.F. 124, p = 0.0019). Dashed line indicates MCS at time of treatment. Statistical analysis was determined using two-way ANOVA Tukey’s multiple comparisons test. Gray symbols in the top panels of FIGS. 23A-23C represent non-responding mice. *p < 0.05; **p < 0.01; ***p < 0.001; ****p < 0.0001. FIG. 23D shows representative histological images of two different regions of spinal cord demonstrating multiple foci of inflammation in the white matter of control mice (H&E staining, top row) and serial section of spinal cord from the same mouse showing multifocal demyelination associated with the areas of inflammation (Luxol fast blue (LFB) staining, bottom row). In contrast, despite having reached a higher peak clinical score, there was an absence of infiltrates in the CNS of AAV8.MOG-treated mice. Certain regions of the spinal cord sections are magnified at right.

FIG. 24A, FIG. 24B, FIG. 24C, FIG. 24D, FIG. 24E, FIG. 24F, FIG. 24G, and FIG. 24H show that therapeutic effects of therapy are enhanced following transient rapamycin immunosuppression. EAE was induced as in FIGS. 23A-23D. FIGS. 24A-24C show that mice developed neurological symptoms. FIG. 24A shows MCS 1.4, tail paralysis with hind-leg paresis (n = 10; final control versus final AAV8.MOG: q = 12.03, D.F. 34, p < 0.0001; peak AAV8.MOG versus final AAV8.MOG: q = 9.95, D.F. 34, p < 0.0001). FIG. 24B shows MCS 3.0, hind-leg paralysis (n = 7-8; final control versus final AAV8.MOG: q = 11, D.F. 14, p < 0.0001; peak AAV8.MOG versus final AAV8.MOG: q = 8.085, D.F. 14, p = 0.0003). FIG. 24C shows MCS 3.5, hind-leg paralysis with forearm paresis (n = 5; final control versus final AAV8.MOG: q = 7.439, D.F. 12, p = 0.0010; peak AAV8.MOG versus final AAV8.MOG: q = 7.123, D.F. 12, p = 0.0014). Mice were intravenously injected with either AAV8.MOG and rapamycin (rapa) or rapamycin alone (control). Clinical scores (mean ± SEM) were recorded. Graphical representation of peak and endpoint MCS are shown above group statistics. Dashed lines indicate MCS at time of treatment. Arrows indicate time of vector and rapamycin injections. Data are representative of at least two repeat experiments. FIG. 24D shows a representative FACS analysis of CD25^{hi}FOXP3⁺ Tregs in blood (isolated from mice in group A) after rapamycin treatment. FIG. 24E shows the percentage of Tregs (mean ± SEM) (post-rapa control versus post-rapa AAV8.MOG: n = 3, t = 3.996, df = 4, p = 0.0162, unpaired two-tailed Student’s t test) and FIG. 24F shows activated CD44⁺ Tregs obtained from peripheral blood at the indicated times (post-rapa control versus post-rapa AAV8.mog: n = 3, q = 5.368, df = 8, p = 0.0219; pre-rapa AAV8.MOG versus post-rapa AAV8.MOG: n = 3, q = 7.698, df = 8, p = 0.0027, two-way ANOVA Tukey’s multiple comparisons test) (pre-EAE = naive mice; pre-Rapa = day 0; post-Rapa = day 10). FIG. 24G shows plasma alanine aminotransferase (ALT) activity from AAV8.MOG-treated and control mice following rapamycin treatment with MCS 3.0 (n=10). FIG. 24H shows plasma alanine aminotransferase (ALT) activity from AAV8.MOG-treated and control mice following rapamycin treatment with MCS 3.5 (n = 10). Statistical analysis was determined for the responders by two-way ANOVA with Tukey’s multiple comparisons test. Plots indicated with gray symbols and smaller circles in the top panels of FIGS. 24A-24C indicate non-responding mice. *p < 0.05; **p < 0.01; ***p < 0.001; ****p < 0.0001.

FIG. 25 shows a gating scheme to identify live CD4⁺CD25⁺FoxP3^{gfp+} cells.

FIG. 26 shows that AAV8.MOG/Rapamycin co-treatment reverses clinical signs of pre-existing disease. EAE was induced as previously described. As mice developed neurological symptoms and presented with complete tail paralysis, they were intravenously injected with either AAV8.MOG/rapamycin or rapamycin alone (control). Clinical scores (mean± SEM) were recorded. Dashed line indicates MCS at time of treatment. Arrows indicate time of vector and rapamycin injections. (n=3/group).

FIG. 27A and FIG. 27B show that AAV.MOG is able to prevent (FIG. 27A) and reverse (FIG. 27B) disease even when induced with multiple immunogenic epitopes simultaneously (MOG₃₅₋₅₅ + MOG₁₁₉₋₁₃₂). AAV.MOG is capable of preventing and reversing EAE induced by multiple MOG epitopes simultaneously. EAE was induced by injecting MOG₃₅₋₅₅ + MOG₁₁₉₋₁₃₂/CFA. Mean clinical scores reported as mean ±SEM. Clinical symptoms were either prevented or significantly lower in the treated group as compared to the control group.

FIG. 28A, FIG. 28B, FIG. 28C and FIG. 28D show that the same AAV.MOG vector is effective in genetically diverse strains of mice with different immunodominant epitopes. AAV.MOG vector is thus effective in mice of a different genetic background. Using DBA-1 (H-2^{q}) mice, AAV8.MOG vector was administered two weeks prior to EAE induction using MOG₇₉₋₉₆ (FIG. 28A) or given after early disease onset (FIG. 28C). Treatment both prevented and reversed clinical symptoms as control mice developed severe EAE and had to be euthanized (FIG. 28B). Compared to control mice, mice administered treatment remained symptom free (FIG. 28A) or quickly recovered (FIG. 28C). FIG. 28D shows areas of inflammation (left) and demyelination (right) in control subjects (top) and subjects treated with AAV.MOG (bottom).

FIGS. 29A-29C show sustained reversal of EAE following geneimmunotherapy and fingolimod treatment. Using 8-week-old Female C57BL/6 mice, EAE was induced with a myelin oligodendrocyte glycoprotein epitope (MOG₃₅₋₅₅). At first sign of clinical disease, mice were injected with a hepatocyte directed viral vector encoding MOG (e.g., AAV.MOG vector) and began daily administration of fingolimod via oral gavage until day 24. Control mice were treated with fingolimod only, without the administration of vector or with Null vector. Initially, all mice treated with fingolimod had recovered from EAE symptoms. At day 24, fingolimod treatment was discontinued, except for the half of the control group receiving treatment which continued receiving fingolimod. FIG. 29A shows the effects of stopping and continuing fingolimod treatment on an AAV-MOG therapy. By day 20 post treatment, all treated mice had recovered. However, after treatment was stopped mice that were only receiving fingolimod relapsed and developed severe EAE. Thus, fingolimod-continued treatment had poor clinical score results, while fingolimod-stopped showed a spike in therapeutic results just before the experiment was stopped at day 40 post-gavage. FIG. 29B shows a fingolimod-only treatment that showed therapeutic results from ~day 32 to day 50 post-gavage. Within days, non-MOG vectored mice treated with fingolimod relapsed and developed severe EAE. Whereas mice treated with MOG vector and fingolimod remained nearly disease free. FIG. 29C shows the synergistic effect of vector and fingolimod. The data demonstrates a synergistic effect of vector and fingolimod that results in a significant long-term reversal of disease upon withdrawal of DMT treatment.

FIG. 30 shows the combination treatment of fingolimod and gene-immunotherapy to treat RR-disease in PLP induce EAE. Fingolimod treatment was administered following the initial remittance of disease. Therapeutic vector expressing PLP transgene was subsequently given 2-weeks later, and fingolimod treatment continued for another 2-weeks. As before, shortly after fingolimod treatment was stopped, non-therapeutically vectored mice quickly developed severe EAE disease, whereas treated mice remained disease free.

FIG. 31 shows prednisolone (PRDL) immunosuppression with AAV.MOG induction of tolerance in C57BL/6 mice, where PRDL and AAV.MOG (or AAV.Null) were co-administered together at disease onset, or a mean clinical score (MCS) of 2.0. AAV.Null group represents the control for monitoring disease progression.

FIG. 32 shows prednisolone (PRDL) immunosuppression after AAV.MOG induction of tolerance in C57BL/6 mice, where PRDL was administered at peak disease (MCS > 3.0), after AAV.MOG (or AAV.Null) was administered at disease onset.

FIGS. 33A-33B show a synergistic effect of Prednisolone (PRDL) immunosuppression with AAV.MOG induction of tolerance in C57BL/6 mice. At MCS <2.5 (FIG. 33A) or MCS ≥2.5 (FIG. 33B) mice received AAV.MOG or AAV.Null. At peak of disease (MCS ≥3.0), experimental mice were started on oral gavage of PRDL (10 mg/kg) every 24 hours for 7 days.

### DETAILED DESCRIPTION

The present disclosure provides methods of administering recombinant AAV vectors having enhanced tolerization properties in combination with an immunosuppresive agent. In particular embodiments, the immunosuppresive agent is a small molecule analog. In particular embodiments, the rAAV vectors encode therapeutic peptides, such as MOG, MBP, and/or PLP. The therapeutic peptides encoded in the disclosed vectors are useful for induction of immunological tolerance. Accordingly, the disclosed vectors are particularly useful for the in vivo induction of immunological tolerance via a liver-directed AAV-based gene therapeutic regimen for treating and/or ameliorating autoimmune disorders such as multiple sclerosis. Further disclosed are pharmaceutical compositions comprising the disclosed rAAV vectors and immunosuppressive agents. Further provided herein are methods for preventing an autoimmune disease (e.g., MS) or inhibiting progression of the disease in a mammal, the method comprising administering to the mammal any one of the disclosed compositions, as well as uses of these compositions as medicaments.

In some embodiments, a rAAV nucleic acid vector described herein comprises inverted terminal repeat sequences (ITRs), such as those derived from a wild-type AAV genome, such as the AAV2 genome. In some embodiments, the rAAV nucleic acid vector further comprises a polynucleotide that includes a nucleic acid segment (also referred to as a heterologous nucleic acid molecule or a transgene) operably linked to a promoter and optionally, other regulatory elements, wherein the ITRs flank the polynucleotide containing the nucleic acid segment. In some embodiments, the ITRs flank a polynucleotide containg two, three, or more than three nucleic acid segments.

In some embodiments, the promoter is a mammalian cell-specific or a mammalian tissue-specific promoter. In some embodiments, the promoter is a promoter that is capable of expressing the nucleic acid segment in one or more cells of a mammalian liver, such as hepatocyte cells. Exemplary hepatocyte specific promoters and enhancer elements include, e.g., albumin, human α1-antitrypsin (hAAT), transthyretin (TTR), HCB and apolipoprotein E (apoE) promoters or enhancer elements.

In some embodiments, the rAAV nucleic acid vector comprises a polynucleotide that comprises a first nucleic acid segment that is at least 95%, at least 98%, at least 99%, or at least 99.5% identical to any one of the sequences of SEQ ID NOs: 1-3, 11, 15, and 17. In some embodiments, the rAAV nucleic acid vector comprises a polynucleotide that comprises any one of the sequences of SEQ ID NOs: 1-3, 11, 15, and 17.

In some embodiments, the polynucleotide comprises a first nucleic acid segment (or sequence) that encodes a first autoimmune disease therapeutic molecule of interest (e.g., an “autoimmune therapeutic molecule”). As used herein, an autoimmune therapeutic molecule includes any antigen (such as a protein, fragment thereof, or a peptide) that contributes to initiation and/or progression of an autoimmune disease. Exemplary autoimmune therapeutic molecules include myelin basic protein (MBP, e.g., for multiple sclerosis), proteolipid protein (PLP, e.g., for multiple sclerosis), myelin oligodendrocyte glycoprotein (MOG, e.g., for multiple sclerosis), myelin-associated glycoprotein (MAG, e.g., for Anti-MAG Peripheral Neuropathy), insulin (e.g., for type 1 diabetes), islet-specific glucose-6-phosphatase catalytic subunit-related protein (IGRP, e.g., for type 1 diabetes), Preproinsulin (e.g., for type 1 diabetes), Glutamic decarboxylase (GAD, e.g., for type 1 diabetes), tyrosine phosphatase like autoantigen (e.g., for type 1 diabetes), insulinoma antigen-2 (e.g., for type 1 diabetes), Islet cell antigen (e.g., for type 1 diabetes), thyroid stimulating hormone (TSH) receptor (e.g., for Graves’ disease), thyrotropin receptor (e.g., for Graves’ disease), Aggrecan (e.g., for rheumatoid arthritis), CD4+T cell epitope (GRVRVNSAY (SEQ ID NO: 33), e.g., for proteoglycan induced arthritis (PGIA) or rheumatoid arthritis), or acetylcholine receptor (e.g., for Myasthenia gravis). In some embodiments, the autoimmune therapeutic molecule of interest is a human protein, such as human myelin basic protein (MBP), a human proteolipid protein (PLP), or a human myelin oligodendrocyte glycoprotein (MOG).

In some embodiments, the polynucleotide comprises a first nucleic acid segment that encodes a first autoimmune disease therapeutic molecule of interest, such as a mammalian myelin basic protein (MBP), proteolipid protein (PLP), or myelin oligodendrocyte glycoprotein (MOG). In some embodiments, the nucleic acid segment encodes a human MBP, a human MOG, or a human PLP. In particular embodiments, a full-length MBP, MOG, and/or PLP is encoded in the polynucleotide. In some embodiments, a full-length mammalian MBP, MOG, and/or PLP is encoded in the polynucleotide.

In particular embodiments, a full-length human MBP, a full-length human MOG, and/or a full-length human PLP is encoded in the polynucleotide. In some embodiments, where the first therapeutic molecule is MOG, the first therapeutic molecule is encoded by any one of SEQ ID NOs: 3 and 15. In some embodiments, where the first therapeutic molecule is PLP, the first therapeutic molecule is encoded by any one of SEQ ID NOs: 2 and 11. In some embodiments, where the first therapeutic molecule is MBP, the first therapeutic molecule is encoded by any one of SEQ ID NOs: 1 and 17.

In some embodiments, the polynucleotide encodes a second nucleic acid segment (or sequence) encoding a second autoimmune disease therapeutic molecule of interest, such as a mammalian myelin basic protein (MBP), proteolipid protein (PLP), or myelin oligodendrocyte glycoprotein (MOG). In some embodiments, the second therapeutic molecule is a MBP or a PLP if the first therapeutic molecule is MOG. In some embodiments, the second therapeutic molecule is a MBP or a MOG if the first therapeutic molecule is PLP. In some embodiments, the second therapeutic molecule is a PLP or a MOG if the first therapeutic molecule is MBP. In some embodiments, where the second therapeutic molecule is MOG, the second therapeutic molecule is encoded by any one of SEQ ID NOs: 3 and 15. In some embodiments, where the second therapeutic molecule is PLP, the second therapeutic molecule is encoded by any one of SEQ ID NOs: 2 and 11. In some embodiments, where the second therapeutic molecule is MBP, the second therapeutic molecule is encoded by any one of SEQ ID NOs: 1 and 17.

In some embodiments, the MOG, the PLP, and/or the MBP comprises a full-length polypeptide. In some embodiments, the second nucleic acid segment encodes a polypeptide, a peptide, a ribozyme, a peptide nucleic acid, an siRNA, an RNAi, an antisense oligonucleotide, an antisense polynucleotide, an antibody, an antigen binding fragment, or any combination thereof. In some embodiments, the second nucleic acid sequence encodes a proteolipid protein, a myelin oligodendrocyte, a glycoprotein, a myelin-associated glycoprotein, insulin, an islet-specific glucose-6-phosphatase catalytic subunit-related protein, a Preproinsulin, a glutamic decarboxylase, a tyrosine phosphatase like autoantigen, an insulinoma antigen-2, an Islet cell antigen, a thyroid stimulating hormone (TSH) receptor, a thyrotropin receptor, an Aggrecan, a CD4+ T cell epitope, a porin, or an acetylcholine receptor.

In some embodiments, the second nucleotide sequence or the second autoimmune disease therapeutic molecule of interest is not necessary for the full therapeutic function of the rAAV.

In some embodiments, the polynucleotide encodes a third nucleic acid segment (or sequence) encoding a third autoimmune disease therapeutic molecule of interest, such as a human myelin basic protein (MBP), proteolipid protein (PLP), or myelin oligodendrocyte glycoprotein (MOG). In some embodiments, the third therapeutic molecule is a MOG, if the first and second therapeutic molecules comprise a MBP and a PLP. In some embodiments, the third therapeutic molecule is a PLP, if the first and second therapeutic molecules comprises a MBP and a MOG. In some embodiments, the third therapeutic molecule is a MBP, if the first and second therapeutic molecule comprises a MOG and a PLP. Thus, in some embodiments, the polynucleotide encodes a MOG, a MBP, and a PLP.

In some embodiments, the MOG, the PLP, and/or the MBP comprises a full-length polypeptide. In some embodiments, the third nucleic acid sequence encodes a polypeptide, a peptide, a ribozyme, a peptide nucleic acid, an siRNA, an RNAi, an antisense oligonucleotide, an antisense polynucleotide, an antibody, an antigen binding fragment, or any combination thereof. In some embodiments, the third nucleic acid sequence encodes a proteolipid protein, a myelin oligodendrocyte, a glycoprotein, a myelin-associated glycoprotein, insulin, an islet-specific glucose-6-phosphatase catalytic subunit-related protein, a Preproinsulin, a glutamic decarboxylase, a tyrosine phosphatase like autoantigen, an insulinoma antigen-2, an Islet cell antigen, a thyroid stimulating hormone (TSH) receptor, a thyrotropin receptor, an Aggrecan, a CD4+ T cell epitope, a porin, or an acetylcholine receptor.

In some embodiments, the third nucleotide sequence or the third autoimmune disease therapeutic molecule of interest is not necessary for the full therapeutic function of the rAAV

cDNA sequences and protein sequences that may be encoded by the transgene are provided below. In some embodiments, the transgene comprises a sequence that is at least 80%, 85%, 90%, 91%, 92%, 93%, 94%, 95%, 96%, 97%, 98%, or 99% identical to any one of the cDNA sequences provided below (SEQ ID NOs: 8, 10, 12, 14, 16, 18, 20, 22, 24, 26, 28, 30, or 32). In some embodiments, the transgene comprises a sequence that is any one of the cDNA sequences provided below (SEQ ID NOs: 8, 10, 12, 14, 16, 18, 20, 22, 24, 26, 28, 30, or 32). In some embodiments, the transgene contains a nucleotide sequence that encodes at least 20, 30, 40, 50, 60, 70, 80, 90, 100, 110, 120, 130, 140, 150, 160, 170, 180, 190, 200, 210, 220, 230, 240 or more contiguous amino acids of any one of the protein sequences provided herein (e.g., any one of SEQ ID NOs: 1, 2, 3, 9, 11, 15, 17, 19, 21, 23, 25, 27, 29, or 31). In some embodiments, the transgene contains a nucleotide sequence that encodes a protein that is at least 80%, 85%, 90%, 91%, 92%, 93%, 94%, 95%, 96%, 97%, 98%, or 99% identical to any one of the protein sequences provided herein (e.g., any one of SEQ ID NOs: 1, 2, 3, 9, 11, 15, 17, 19, 21, 23, 25, 27, 29, or 31). In some embodiments, the transgene contains a nucleotide sequence that encodes any one of the protein sequences provided herein (e.g., any one of SEQ ID NOs: 1, 2, 3, 9, 11, 15, 17, 19, 21, 23, 25, 27, 29, or 31).

Exemplary Mus musculus myelin oligodendrocyte glycoprotein (MOG) cDNA

1 atggcctgtt tgtggagctt ctctttgccc agctgcttcc tctcccttct cctcctcctt
61 ctcctccagt tgtcatgcag ctatgcagga caattcagag tgataggacc agggtatccc
121 atccgggctt tagttgggga tgaagcagag ctgccgtgcc gcatctctcc tgggaaaaat
181 gccacgggca tggaggtggg ttggtaccgt tctcccttct caagagtggt tcacctctac
241 cgaaatggca aggaccaaga tgcagagcaa gcacctgaat accggggacg cacagagctt
301 ctgaaagaga ctatcagtga gggaaaggtt acccttagga ttcagaacgt gagattctca
361 gatgaaggag gctacacctg cttcttcaga gaccactctt accaagaaga ggcagcaatg
421 gagttgaaag tggaagatcc cttctattgg gtcaaccccg gtgtgctgac tctcatcgca
481 cttgtgccta cgatcctcct gcaggtctct gtaggccttg tattcctctt cctgcagcac
541 agactgagag gaaaacttcg tgcagaagta gagaatctcc atcggacttt tgatcctcac
601 ttcctgaggg tgccctgctg gaagataaca ctgtttgtta ttgtgcctgt tcttggaccc
661 ctggttgcct tgatcatctg ctacaactgg ctgcaccgaa gactggcagg acagtttctt
721 gaagagctaa gaaaccccct ttga (SEQ ID NO: 8)

Exemplary mus musculus myelin-oligodendrocyte glycoprotein (MOG) protein

1 mac1wsfswp scf1s11111 11q1scsyag qfrvigpgyp iralvgdeae 1pcrispgkn
61 atgmevgwyr spfsrvvhly rngkdqdaeq apeyrgrtel lketisegkv tlriqnvrfs
121 deggytcffr dhsyqeeaam elkvedpfyw vnpgvltlia lvptillqvs vglvflflqh
181 rlrgklraev enlhrtfdph flrvpcwkit lfvivpvlgp lvaliicynw lhrrlagqfl
241 eelrnpf (SEQ ID NO: 9)

Exemplary Mus musculus proteolipid protein 1 (PLP) cDNA

ATGGGCTTGTTAGAGTGTTGTGCTAGATGTCTGGTAGGGGCCCCCTTTGC
TTCCCTGGTGGCCACTGGATTGTGTTTCTTTGGAGTGGCACTGTTCTGTG
GATGTGGACATGAAGCTCTCACTGGTACAGAAAAGCTAATTGAGACCTAT
TTCTCCAAAAACTACCAGGACTATGAGTATCTCATTAATGTGATTCATGC
TTTCCAGTATGTCATCTATGGAACTGCCTCTTTCTTCTTCCTTTATGGGG
CCCTCCTGCTGGCTGAGGGCTTCTACACCACCGGCGCTGTCAGGCAGATC
TTTGGCGACTACAAGACCACCATCTGCGGCAAGGGCCTGAGCGCAACGGT
AACAGGGGGCCAGAAGGGGAGGGGTTCCAGAGGCCAACATCAAGCTCATT
CTTTGGAGCGGGTGTGTCATTGTTTGGGAAAATGGCTAGGACATCCCGAC
AAGTTTGTGGGCATCACCTATGCCCTGACTGTTGTATGGCTCCTGGTGTT
TGCCTGCTCGGCTGTACCTGTGTACATTTACTTCAATACCTGGACCACCT
GTCAGTCTATTGCCTTCCCTAGCAAGACCTCTGCCAGTATAGGCAGTCTC
TGCGCTGATGCCAGAATGTATGGTGTTCTCCCATGGAATGCTTTCCCTGG
CAAGGTTTGTGGCTCCAACCTTCTGTCCATCTGCAAAACAGCTGAGTTCC
AAATGACCTTCCACCTGTTTATTGCTGCGTTTGTGGGTGCTGCGGCCACA
CTAGTTTCCCTGCTCACCTTCATGATTGCTGCCACTTACAACTTCGCCGT
CCTTAAACTCATGGGCCGAGGCACCAAGTTCTGA (SEQ IDNO: 10)

Exemplary Mus musculus proteolipid protein 1 (PLP) protein

1 mglleccarc lvgapfaslv atglcffgva lfcgcgheal tgtekliety fsknyqdyey
61 linvihafqy viygtasfff lygalllaeg fyttgavrqi fgdyktticg kglsatvtgg
121 qkgrgsrgqh qahslervch clgkwlghpd kfvgityalt vvwllvfacs avpvyiyfnt
181 wttcqsiafp sktsasigsl cadarmygvl pwnafpgkvc gsnllsickt aefqmtfhlf
241 iaafvgaaat lvslltfmia atynfavlkl mgrgtkf (SEQ ID NO: 11)

Exemplary Mus musculus myelin basic protein (MBP) cDNA

ATGGGAAACCACTCTGGAAAGAGAGAATTATCTGCTGAGAAGGCCAGTAA
GGATGGAGAGATTCACCGAGGAGAGGCTGGAAAGAAGAGAAGCGTGGGCA
AGCTTTCTCAGACGGCCTCAGAGGACAGTGATGTGTTTGGGGAGGCAGAT
GCGATCCAGAACAATGGGACCTCGGCTGAGGACACGGCGGTGACAGACTC
CAAGCACACAGCAGACCCAAAGAATAACTGGCAAGGCGCCCACCCAGCTG
ACCCAGGGAACCGCCCCCACTTGATCCGCCTCTTTTCCCGAGATGCCCCG
GGAAGGGAGGACAACACCTTCAAAGACAGGCCCTCAGAGTCCGACGAGCT
TCAGACCATCCAAGAAGACCCCACAGCAGCTTCCGGAGGCCTGGATGTGA
TGGCATCACAGAAGAGACCCTCACAGCGATCCAAGTACCTGGCCACAGCA
AGTACCATGGACCATGCCAGGCATGGCTTCCTCCCAAGGCACAGAGACAC
GGGCATCCTTGACTCCATCGGGCGCTTCTTTAGCGGTGACAGGGGTGCGC
CCAAGCGGGGCTCTGGCAAGGTGAGCTCCGAGCCGTAG (SEQ ID NO:
12)

Exemplary Mus musculus myelin basic protein (MBP) protein

1 mgnhsgkrel saekaskdge ihrgeagkkr svgklsqtas edsdvfgead aiqnngtsae
61 dtavtdskht adpknnwqga hpadpgnrph lirlfsrdap gredntfkdr psesdelqti
121 qedptaasgg ldvmasqkrp sqrskylata stmdharhgf lprhrdtgil dsigrffsgd
181 rgapkrgsgk vssep (SEQ ID NO: 1)

Exemplary Homo sapiens myelin oligodendrocyte glycoprotein (MOG) cDNA

1 atggcaagct tatcgagacc ctctctgccc agctgcctct gctccttcct cctcctcctc
61 ctcctccaag tgtcttccag ctatgcaggg cagttcagag tgataggacc aagacaccct
121 atccgggctc tggtcgggga tgaagtggaa ttgccatgtc gcatatctcc tgggaagaac
181 gctacaggca tggaggtggg gtggtaccgc ccccccttct ctagggtggt tcatctctac
241 agaaatggca aggaccaaga tggagaccag gcacctgaat atcggggccg gacagagctg
301 ctgaaagatg ctattggtga gggaaaggtg actctcagga tccggaatgt aaggttctca
361 gatgaaggag gtttcacctg cttcttccga gatcattctt accaagagga ggcagcaatg
421 gaattgaaag tagaagatcc tttctactgg gtgagccctg gagtgctggt tctcctcgcg
481 gtgctgcctg tgctcctcct gcagatcact gttggcctcg tcttcctctg cctgcagtac
541 agactgagag gaaaacttcg agcagagata gagaatctcc accggacttt tgatccccac
601 tttctgaggg tgccctgctg gaagataacc ctgtttgtaa ttgtgccggt tcttggaccc
661 ttggttgcct tgatcatctg ctacaactgg ctacatcgaa gactagcagg gcaattcctt
721 gaagagctac gaaatccctt ctga (SEQ ID NO: 14)

Exemplary Homo sapiens myelin oligodendrocyte glycoprotein (MOG) protein

1 maslsrpslp sclcsfllll llqvsssyag qfrvigprhp iralvgdeve lpcrispgkn
61 atgmevgwyr ppfsrvvhly rngkdqdgdq apeyrgrtel lkdaigegkv tlrirnvrfs
121 deggftcffr dhsyqeeaam elkvedpfyw vspgvlvlla vlpvlllqit vglvflclqy
181 rlrgklraei enlhrtfdph flrvpcwkit lfvivpvlgp lvaliicynw lhrrlagqfl
241 eelrnpf (SEQ ID NO: 15)

Exemplary Homo sapiens myelin basic protein (MBP), transcript variant 7, cDNA

ATGGGAAACCACGCAGGCAAACGAGAATTAAATGCCGAGAAGGCCAGTAC
GAATAGTGAAACTAACAGAGGAGAATCTGAAAAAAAGAGAAACCTGGGTG
AACTTTCACGGACAACCTCAGAGGACAACGAAGTGTTCGGAGAGGCAGAT
GCGAACCAGAACAATGGGACCTCCTCTCAGGACACAGCGGTGACTGACTC
CAAGCGCACAGCGGACCCGAAGAATGCCTGGCAGGATGCCCACCCAGCTG
ACCCAGGGAGCCGCCCCCACTTGATCCGCCTCTTTTCCCGAGATGCCCCG
GGGAGGGAGGACAACACCTTCAAAGACAGGCCCTCTGAGTCCGACGAGCT
CCAGACCATCCAAGAAGACAGTGCAGCCACCTCCGAGAGCCTGGATGTGA
TGGCGTCACAGAAGAGACCCTCCCAGAGGCACGGATCCAAGTACCTGGCC
ACAGCAAGTACCATGGACCATGCCAGGCATGGCTTCCTCCCAAGGCACAG
AGACACGGGCATCCTTGACTCCATCGGGCGCTTCTTTGGCGGTGACAGGG
GTGCGCCCAAGCGGGGCTCTGGCAAGGACTCACACCACCCGGCAAGAACT
GCTCACTACGGCTCCCTGCCCCAGAAGTCACACGGCCGGACCCAAGATGA
AAACCCCGTAGTCCACTTCTTCAAGAACATTGTGACGCCTCGCACACCAC
CCCCGTCGCAGGGAAAGGGGAGAGGACTGTCCCTGAGCAGATTTAGCTGG
GGGGCCGAAGGCCAGAGACCAGGATTTGGCTACGGAGGCAGAGCGTCCGA
CTATAAATCGGCTCACAAGGGATTCAAGGGAGTCGATGCCCAGGGCACGC
TTTCCAAAATTTTTAAGCTGGGAGGAAGAGATAGTCGCTCTGGATCACCC
ATGGCTAGACGCTGA (SEQ ID NO: 16)

Exemplary Homo sapiens myelin basic protein (MBP), transcript variant 7, protein

1 mgnhagkrel naekastnse tnrgesekkr nlgelsrtts ednevfgead anqnngtssq
61 dtavtdskrt adpknawqda hpadpgsrph lirlfsrdap gredntfkdr psesdelqti
121 qedsaatses ldvrnasqkrp sqrhgskyla tastmdharh gflprhrdtg ildsigrffg
181 gdrgapkrgs gkdshhpart ahygslpqks hgrtqdenpv vhffknivtp rtpppsqgkg
241 rglslsrfsw gaegqrpgfg yggrasdyks ahkgfkgvda qgtlskifkl ggrdsrsgsp
301 marr (SEQ ID NO: 17)

Exemplary Homo sapiens myelin basic protein (MBP), transcript variant 1, cDNA

ATGGCGTCACAGAAGAGACCCTCCCAGAGGCACGGATCCAAGTACCTGGC
CACAGCAAGTACCATGGACCATGCCAGGCATGGCTTCCTCCCAAGGCACA
GAGACACGGGCATCCTTGACTCCATCGGGCGCTTCTTTGGCGGTGACAGG
GGTGCGCCCAAGCGGGGCTCTGGCAAGGTACCCTGGCTAAAGCCGGGCCG
GAGCCCTCTGCCCTCTCATGCCCGCAGCCAGCCTGGGCTGTGCAACATGT
ACAAGGACTCACACCACCCGGCAAGAACTGCTCACTACGGCTCCCTGCCC
CAGAAGTCACACGGCCGGACCCAAGATGAAAACCCCGTAGTCCACTTCTT
CAAGAACATTGTGACGCCTCGCACACCACCCCCGTCGCAGGGAAAGGGGA
GAGGACTGTCCCTGAGCAGATTTAGCTGGGGGGCCGAAGGCCAGAGACCA
GGATTTGGCTACGGAGGCAGAGCGTCCGACTATAAATCGGCTCACAAGGG
ATTCAAGGGAGTCGATGCCCAGGGCACGCTTTCCAAAATTTTTAAGCTGG
GAGGAAGAGATAGTCGCTCTGGATCACCCATGGCTAGACGCTGA (SEQ
ID NO: 18)

Exemplary Homo sapiens myelin basic protein (MBP), transcript variant 1, protein

1 masqkrpsqr hgskylatas tmdharhgfl prhrdtgild sigrffggdr gapkrgsgkv
61 pwlkpgrspl psharsqpgl cnmykdshhp artahygslp qkshgrtqde npvvhffkni
121 vtprtpppsq gkgrglslsr fswgaegqrp gfgyggrasd yksahkgfkg vdaqgtlski
181 fklggrdsrs gspmarr (SEQ ID NO: 19)

Exemplary Homo sapiens myelin basic protein (MBP), transcript variant 2, cDNA

ATGGCGTCACAGAAGAGACCCTCCCAGAGGCACGGATCCAAGTACCTGGC
CACAGCAAGTACCATGGACCATGCCAGGCATGGCTTCCTCCCAAGGCACA
GAGACACGGGCATCCTTGACTCCATCGGGCGCTTCTTTGGCGGTGACAGG
GGTGCGCCCAAGCGGGGCTCTGGCAAGGTACCCTGGCTAAAGCCGGGCCG
GAGCCCTCTGCCCTCTCATGCCCGCAGCCAGCCTGGGCTGTGCAACATGT
ACAAGGACTCACACCACCCGGCAAGAACTGCTCACTACGGCTCCCTGCCC
CAGAAGTCACACGGCCGGACCCAAGATGAAAACCCCGTAGTCCACTTCTT
CAAGAACATTGTGACGCCTCGCACACCACCCCCGTCGCAGGGAAAGGGGG
CCGAAGGCCAGAGACCAGGATTTGGCTACGGAGGCAGAGCGTCCGACTAT
AAATCGGCTCACAAGGGATTCAAGGGAGTCGATGCCCAGGGCACGCTTTC
CAAAATTTTTAAGCTGGGAGGAAGAGATAGTCGCTCTGGATCACCCATGG
CTAGACGCTGA (SEQ ID NO: 20)

Exemplary Homo sapiens myelin basic protein (MBP), transcript variant 2, protein

1 masqkrpsqr hgskylatas tmdharhgfl prhrdtgild sigrffggdr gapkrgsgkv
61 pwlkpgrspl psharsqpgl cnmykdshhp artahygslp qkshgrtqde npvvhffkni
121 vtprtpppsq gkgaegqrpg fgyggrasdy ksahkgfkgv daqgtlskif klggrdsrsg
181 spmarr (SEQ ID NO: 21)

Exemplary Homo sapiens myelin basic protein (MBP), transcript variant 3, cDNA

ATGGCGTCACAGAAGAGACCCTCCCAGAGGCACGGATCCAAGTACCTGGC
CACAGCAAGTACCATGGACCATGCCAGGCATGGCTTCCTCCCAAGGCACA
GAGACACGGGCATCCTTGACTCCATCGGGCGCTTCTTTGGCGGTGACAGG
GGTGCGCCCAAGCGGGGCTCTGGCAAGGACTCACACCACCCGGCAAGAAC
TGCTCACTACGGCTCCCTGCCCCAGAAGTCACACGGCCGGACCCAAGATG
AAAACCCCGTAGTCCACTTCTTCAAGAACATTGTGACGCCTCGCACACCA
CCCCCGTCGCAGGGAAAGGGGAGAGGACTGTCCCTGAGCAGATTTAGCTG
GGGGGCCGAAGGCCAGAGACCAGGATTTGGCTACGGAGGCAGAGCGTCCG
ACTATAAATCGGCTCACAAGGGATTCAAGGGAGTCGATGCCCAGGGCACG
CTTTCCAAAATTTTTAAGCTGGGAGGAAGAGATAGTCGCTCTGGATCACC
CATGGCTAGACGCTGA (SEQ ID NO: 22)

Exemplary Homo sapiens myelin basic protein (MBP), transcript variant 3, protein

1 masqkrpsqr hgskylatas tmdharhgfl prhrdtgild sigrffggdr gapkrgsgkd
61 shhpartahy gslpqkshgr tqdenpvvhf fknivtprtp ppsqgkgrgl slsrfswgae
121 gqrpgfgygg rasdyksahk gfkgvdaqgt lskifklggr dsrsgspmar r (SEQ ID NO: 23)

Exemplary Homo sapiens myelin basic protein (MBP), transcript variant 4, cDNA

ATGGCGTCACAGAAGAGACCCTCCCAGAGGCACGGATCCAAGTACCTGGC
CACAGCAAGTACCATGGACCATGCCAGGCATGGCTTCCTCCCAAGGCACA
GAGACACGGGCATCCTTGACTCCATCGGGCGCTTCTTTGGCGGTGACAGG
GGTGCGCCCAAGCGGGGCTCTGGCAAGGACTCACACCACCCGGCAAGAAC
TGCTCACTACGGCTCCCTGCCCCAGAAGTCACACGGCCGGACCCAAGATG
AAAACCCCGTAGTCCACTTCTTCAAGAACATTGTGACGCCTCGCACACCA
CCCCCGTCGCAGGGAAAGGGGGCCGAAGGCCAGAGACCAGGATTTGGCTA
CGGAGGCAGAGCGTCCGACTATAAATCGGCTCACAAGGGATTCAAGGGAG
TCGATGCCCAGGGCACGCTTTCCAAAATTTTTAAGCTGGGAGGAAGAGAT
AGTCGCTCTGGATCACCCATGGCTAGACGCTGA (SEQ IDNO: 24)

Exemplary Homo sapiens myelin basic protein (MBP), transcript variant 4, protein

1 masqkrpsqr hgskylatas tmdharhgfl prhrdtgild sigrffggdr gapkrgsgkd
61 shhpartahy gslpqkshgr tqdenpvvhf fknivtprtp ppsqgkgaeg qrpgfgyggr
121 asdyksahkg fkgvdaqgtl skifklggrd srsgspmarr (SEQ ID NO: 25)

Exemplary Homo sapiens myelin basic protein (MBP), transcript variant 8, cDNA

ATGGGAAACCACGCAGGCAAACGAGAATTAAATGCCGAGAAGGCCAGTAC
GAATAGTGAAACTAACAGAGGAGAATCTGAAAAAAAGAGAAACCTGGGTG
AACTTTCACGGACAACCTCAGAGGACAACGAAGTGTTCGGAGAGGCAGAT
GCGAACCAGAACAATGGGACCTCCTCTCAGGACACAGCGGTGACTGACTC
CAAGCGCACAGCGGACCCGAAGAATGCCTGGCAGGATGCCCACCCAGCTG
ACCCAGGGAGCCGCCCCCACTTGATCCGCCTCTTTTCCCGAGATGCCCCG
GGGAGGGAGGACAACACCTTCAAAGACAGGCCCTCTGAGTCCGACGAGCT
CCAGACCATCCAAGAAGACAGTGCAGCCACCTCCGAGAGCCTGGATGTGA
TGGCGTCACAGAAGAGACCCTCCCAGAGGCACGGATCCAAGTACCTGGCC
ACAGCAAGTACCATGGACCATGCCAGGCATGGCTTCCTCCCAAGGCACAG
AGACACGGGCATCCTTGACTCCATCGGGCGCTTCTTTGGCGGTGACAGGG
GTGCGCCCAAGCGGGGCTCTGGCAAGGTGAGCTCTGAGGAGTAG (SEQ
ID NO: 26)

Exemplary Homo sapiens myelin basic protein (MBP), transcript variant 8, protein

1 mgnhagkrel naekastnse tnrgesekkr nlgelsrtts ednevfgead anqnngtssq
61 dtavtdskrt adpknawqda hpadpgsrph lirlfsrdap gredntfkdr psesdelqti
121 qedsaatses ldvrnasqkrp sqrhgskyla tastmdharh gflprhrdtg ildsigrffg
181 gdrgapkrgs gkvssee (SEQ ID NO: 27)

Exemplary Homo sapiens proteolipid protein 1 (PLP1), transcript variant 1, cDNA

ATGGGCTTGTTAGAGTGCTGTGCAAGATGTCTGGTAGGGGCCCCCTTTGC
TTCCCTGGTGGCCACTGGATTGTGTTTCTTTGGGGTGGCACTGTTCTGTG
GCTGTGGACATGAAGCCCTCACTGGCACAGAAAAGCTAATTGAGACCTAT
TTCTCCAAAAACTACCAAGACTATGAGTATCTCATCAATGTGATCCATGC
CTTCCAGTATGTCATCTATGGAACTGCCTCTTTCTTCTTCCTTTATGGGG
CCCTCCTGCTGGCTGAGGGCTTCTACACCACCGGCGCAGTCAGGCAGATC
TTTGGCGACTACAAGACCACCATCTGCGGCAAGGGCCTGAGCGCAACGGT
AACAGGGGGCCAGAAGGGGAGGGGTTCCAGAGGCCAACATCAAGCTCATT
CTTTGGAGCGGGTGTGTCATTGTTTGGGAAAATGGCTAGGACATCCCGAC
AAGTTTGTGGGCATCACCTATGCCCTGACCGTTGTGTGGCTCCTGGTGTT
TGCCTGCTCTGCTGTGCCTGTGTACATTTACTTCAACACCTGGACCACCT
GCCAGTCTATTGCCTTCCCCAGCAAGACCTCTGCCAGTATAGGCAGTCTC
TGTGCTGATGCCAGAATGTATGGTGTTCTCCCATGGAATGCTTTCCCTGG
CAAGGTTTGTGGCTCCAACCTTCTGTCCATCTGCAAAACAGCTGAGTTCC
AAATGACCTTCCACCTGTTTATTGCTGCATTTGTGGGGGCTGCAGCTACA
CTGGTTTCCCTGCTCACCTTCATGATTGCTGCCACTTACAACTTTGCCGT
CCTTAAACTCATGGGCCGAGGCACCAAGTTCTGA (SEQ IDNO: 28)

Exemplary Homo sapiens proteolipid protein 1 (PLP1), transcript variant 1, protein

1 mglleccarc lvgapfasIv atglcffgva lfcgcgheal tgtekliety fsknyqdyey
61 linvihafqy viygtasfff lygalllaeg fyttgavrqi fgdyktticg kglsatvtgg
121 qkgrgsrgqh qahslervch clgkwlghpd kfvgityalt vvwllvfacs avpvyiyfnt
181 wttcqsiafp sktsasigsl cadarmygvl pwnafpgkvc gsnllsickt aefqmtfhlf
241 iaafvgaaat lvslltfmia atynfavlkl mgrgtkf (SEQ ID NO: 11)

Exemplary Homo sapiens proteolipid protein 1 (PLP1), transcript variant 2, cDNA

ATGGGCTTGTTAGAGTGCTGTGCAAGATGTCTGGTAGGGGCCCCCTTTGC
TTCCCTGGTGGCCACTGGATTGTGTTTCTTTGGGGTGGCACTGTTCTGTG
GCTGTGGACATGAAGCCCTCACTGGCACAGAAAAGCTAATTGAGACCTAT
TTCTCCAAAAACTACCAAGACTATGAGTATCTCATCAATGTGATCCATGC
CTTCCAGTATGTCATCTATGGAACTGCCTCTTTCTTCTTCCTTTATGGGG
CCCTCCTGCTGGCTGAGGGCTTCTACACCACCGGCGCAGTCAGGCAGATC
TTTGGCGACTACAAGACCACCATCTGCGGCAAGGGCCTGAGCGCAACGTT
GTGGGCATCACCTATGCCCTGACCGTTGTGTGGCTCCTGGTGTTTGCCTG
CTCTGCTGTGCCTGTGTACATTTACTTCAACACCTGGACCACCTGCCAGT
CTATTGCCTTCCCCAGCAAGACCTCTGCCAGTATAGGCAGTCTCTGTGCT
GATGCCAGAATGTATGGTGTTCTCCCATGGAATGCTTTCCCTGGCAAGGT
TTGTGGCTCCAACCTTCTGTCCATCTGCAAAACAGCTGAGTTCCAAATGA
CCTTCCACCTGTTTATTGCTGCATTTGTGGGGGCTGCAGCTACACTGGTT
TCCCTGCTCACCTTCATGATTGCTGCCACTTACAACTTTGCCGTCCTTAA
ACTCATGGGCCGAGGCACCAAGTTCTGA (SEQ ID NO: 30)

Exemplary Homo sapiens proteolipid protein 1 (PLP1), transcript variant 2, protein

1 mglleccarc lvgapfaslv atglcffgva lfcgcgheal tgtekliety fsknyqdyey
61 linvihafqy viygtasfff lygalllaeg fyttgavrqi fgdyktticg kglsatfvgi
121 tyaltvvwll vfacsavpvy iyfntwttcq siafpsktsa sigslcadar mygvlpwnaf
181 pgkvcgsnll sicktaefqm tfhifiaafv gaaatlvsll tfmiaatynf avlklmgrgt
241 kf (SEQ ID NO: 31)

Exemplary Homo sapiens proteolipid protein 1 (PLP1), transcript variant 3, cDNA

ATGGGCTTGTTAGAGTGCTGTGCAAGATGTCTGGTAGGGGCCCCCTTTGC
TTCCCTGGTGGCCACTGGATTGTGTTTCTTTGGGGTGGCACTGTTCTGTG
GCTGTGGACATGAAGCCCTCACTGGCACAGAAAAGCTAATTGAGACCTAT
TTCTCCAAAAACTACCAAGACTATGAGTATCTCATCAATGTGATCCATGC
CTTCCAGTATGTCATCTATGGAACTGCCTCTTTCTTCTTCCTTTATGGGG
CCCTCCTGCTGGCTGAGGGCTTCTACACCACCGGCGCAGTCAGGCAGATC
TTTGGCGACTACAAGACCACCATCTGCGGCAAGGGCCTGAGCGCAACGGT
AACAGGGGGCCAGAAGGGGAGGGGTTCCAGAGGCCAACATCAAGCTCATT
CTTTGGAGCGGGTGTGTCATTGTTTGGGAAAATGGCTAGGACATCCCGAC
AAGTTTGTGGGCATCACCTATGCCCTGACCGTTGTGTGGCTCCTGGTGTT
TGCCTGCTCTGCTGTGCCTGTGTACATTTACTTCAACACCTGGACCACCT
GCCAGTCTATTGCCTTCCCCAGCAAGACCTCTGCCAGTATAGGCAGTCTC
TGTGCTGATGCCAGAATGTATGGTGTTCTCCCATGGAATGCTTTCCCTGG
CAAGGTTTGTGGCTCCAACCTTCTGTCCATCTGCAAAACAGCTGAGTTCC
AAATGACCTTCCACCTGTTTATTGCTGCATTTGTGGGGGCTGCAGCTACA
CTGGTTTCCCTGCTCACCTTCATGATTGCTGCCACTTACAACTTTGCCGT
CCTTAAACTCATGGGCCGAGGCACCAAGTTCTGA (SEQ IDNO: 28)

Exemplary Homo sapiens proteolipid protein 1 (PLP1), transcript variant 3, protein

1 mglleccarc lvgapfaslv atglcffgva lfcgcgheal tgtekliety fsknyqdyey
61 linvihafqy viygtasfff lygalllaeg fyttgavrqi fgdyktticg kglsatvtgg
121 qkgrgsrgqh qahslervch clgkwlghpd kfvgityalt vvwllvfacs avpvyiyfnt
181 wttcqsiafp sktsasigsl cadarmygvl pwnafpgkvc gsnllsickt aefqmtfhlf
241 iaafvgaaat lvslltfmia atynfavlkl mgrgtkf (SEQ ID NO: 11)

Exemplary Homo sapiens proteolipid protein 1 (PLP1), transcript variant 4, cDNA

ATGGACTATGAGTATCTCATCAATGTGATCCATGCCTTCCAGTATGTCAT
CTATGGAACTGCCTCTTTCTTCTTCCTTTATGGGGCCCTCCTGCTGGCTG
AGGGCTTCTACACCACCGGCGCAGTCAGGCAGATCTTTGGCGACTACAAG
ACCACCATCTGCGGCAAGGGCCTGAGCGCAACGGTAACAGGGGGCCAGAA
GGGGAGGGGTTCCAGAGGCCAACATCAAGCTCATTCTTTGGAGCGGGTGT
GTCATTGTTTGGGAAAATGGCTAGGACATCCCGACAAGTTTGTGGGCATC
ACCTATGCCCTGACCGTTGTGTGGCTCCTGGTGTTTGCCTGCTCTGCTGT
GCCTGTGTACATTTACTTCAACACCTGGACCACCTGCCAGTCTATTGCCT
TCCCCAGCAAGACCTCTGCCAGTATAGGCAGTCTCTGTGCTGATGCCAGA
ATGTATGGTGTTCTCCCATGGAATGCTTTCCCTGGCAAGGTTTGTGGCTC
CAACCTTCTGTCCATCTGCAAAACAGCTGAGTTCCAAATGACCTTCCACC
TGTTTATTGCTGCATTTGTGGGGGCTGCAGCTACACTGGTTTCCCTGCTC
ACCTTCATGATTGCTGCCACTTACAACTTTGCCGTCCTTAAACTCATGGG
CCGAGGCACCAAGTTCTGA (SEQ ID NO: 32)

Exemplary Homo sapiens proteolipid protein 1 (PLP1), transcript variant 4, protein

1 mdyeylinvi hafqyviygt asffflygal llaegfyttg avrqifgdyk tticgkglsa
61 tvtggqkgrg srgqhqahsl ervchclgkw 1ghpdkfvgi tyaltvvwll vfacsavpvy
121 iyfntwttcq siafpsktsa sigslcadar mygvlpwnaf pgkvcgsnll sicktaefqm
181 tfhifiaafv gaaatlvsll tfmiaatynf avlklmgrgt kf (SEQ ID NO: 29)

In some embodiments, the polynucleotide comprises a third nucleic acid segment (or sequence) encoding a third therapeutic molecule. In some embodiments, the third therapeutic molecule is MOG, if the first and second therapeutic molecules comprise MBP and PLP. In some embodiments, the third therapeutic molecule is PLP, if the first and second therapeutic molecules comprises MBP and MOG. In some embodiments, the third therapeutic molecule is MBP, if the first and second therapeutic molecule comprises MOG and PLP. In some embodiments, the polynucleotide encodes MOG, MBP, and PLP.

In some embodiments, the third nucleotide sequence or the second autoimmune disease therapeutic molecule of interest is not necessary for the full therapeutic function of the rAAV.

In some embodiments, the second therapeutic molecule and/or the third therapeutic molecule comprises an amino acid sequence as set forth in SEQ ID NO:1, SEQ ID NO:2, or SEQ ID NO:3. In some embodiments, the MOG, the PLP, and/or the MBP comprises a full-length polypeptide.

Some embodiments contemplate a pharmaceutical composition for treating or ameliorating one or more symptoms of an autoimmune disease in a mammal, that comprises an effective amount of the rAAV vector as described herein.

Some embodiments contemplate a method of treating a mammal in need thereof (e.g., a human subject) comprising systemically administering to the mammal a therapeutically-effective amount of the rAAV vector as described herein or the pharmaceutical composition as described herein.

Some embodiments contemplate a method for preventing an autoimmune disease or inhibiting progression of the disease in a mammal, the method comprising systemically administering to the mammal, the rAAV vector as described herein or the pharmaceutical composition as described herein in an amount and for a time sufficient to prevent or inhibit progression of the autoimmune disease in the mammal.

In some embodiments, the mammal (e.g., a human mammal) has, is suspected of having, is at risk for developing, or has been diagnosed with the autoimmune disease. In some embodiments, the autoimmune disease is multiple sclerosis, disseminated sclerosis, encephalomyelitis disseminata, optic neuritis, celiac disease, or an allergic disease. Uses of any of the disclosed compositions as a medicament to treat multiple sclerosis, disseminated sclerosis, encephalomyelitis disseminata, optic neuritis, celiac disease, or an allergic disease are als contemplated. In some embodiments, the mammal is a newborn, an infant, a juvenile, an adult, or a young adult.

In some embodiments, expression of the therapeutic molecule in the mammal reduces CNS inflammation, inhibits demyelination, re-establishes immune tolerance to one or more neuroproteins, stimulates the production of endogenous antigen-specific regulatory T cells, or any combination thereof. In some embodiments, the autoimmune disease is multiple sclerosis. In some embodiments, progression of the autoimmune disease in the mammal is inhibited or reversed for at least 50 days, at least 75 days, at least 100 days, at least 125 days, at least 150 days, at least 175 days, at least 200 days, or more than 200 days after administration of the rAAV vector. In some embodiments, progression of the autoimmune disease in the mammal is inhibited or reversed for at least 150 days after administration of the rAAV vector.

In some embodiments, the rAAV vector or the pharmaceutical composition is able to provide therapeutic results following administration to the mammal after a single injection (e.g., a single systemic injection) of vector. In particular embodiments, the injection comprises less than 10¹³, less than 10¹², or less than 10¹¹ vector genomes/ml of rAAV vector.

In some embodiments, expression of the therapeutic molecule in the mammal re-establishes immune tolerance to at least two different neuroprotein epitopes. In some embodiments, the at least two different neuroprotein epitopes comprise different epitopes of a single neuroprotein, e.g. a MOG protein.

Some embodiments contemplate the use of the rAAV vector as disclosed herein, or the pharmaceutical composition vector as disclosed herein as a medicament. Some embodiments contemplate the rAAV vector as disclosed herein, or the pharmaceutical composition vector as disclosed herein for use in treating or ameliorating one or more symptoms of multiple sclerosis in a mammal.

In certain embodiments, the nucleic acid segments cloned into the novel rAAV expression vectors described herein will express or encode one or more polypeptides, peptides, ribozymes, peptide nucleic acids, siRNA’s, RNAi’s, antisense oligonucleotides, antisense polynucleotides, antibodies, antigen binding fragments, or any combination thereof.

As noted herein, the therapeutic agents useful in the present disclosure may include one or more agonists, antagonists, anti-apoptosis factors, inhibitors, receptors, cytokines, cytotoxins, erythropoietic agents, glycoproteins, growth factors, growth factor receptors, hormones, hormone receptors, interferons, interleukins, interleukin receptors, nerve growth factors, neuroactive peptides, neuroactive peptide receptors, proteases, protease inhibitors, protein decarboxylases, protein kinases, protein kinase inhibitors, enzymes, receptor binding proteins, transport proteins or one or more inhibitors thereof, serotonin receptors, or one or more uptake inhibitors thereof, serpins, serpin receptors, tumor suppressors, diagnostic molecules, chemotherapeutic agents, cytotoxins, or any combination thereof.

In related embodiments, the present disclosure further provides populations and pluralities of rAAV nucleic acid vectors, virions, infectious viral particles, or host cells that include one or more nucleic acid segments that encode an autoimmune disease therapeutic agent.

In some embodiments, the second and/or third nucleic acid sequence encodes a polypeptide, a peptide, a ribozyme, a peptide nucleic acid, an siRNA, an RNAi, an antisense oligonucleotide, an antisense polynucleotide, an antibody, an antigen binding fragment, or any combination thereof. In some embodiments, the second and/or third nucleic acid sequence encodes a proteolipid protein, a myelin oligodendrocyte, a glycoprotein, a myelin-associated glycoprotein, insulin, an islet-specific glucose-6-phosphatase catalytic subunit-related protein, a Preproinsulin, a glutamic decarboxylase, a tyrosine phosphatase like autoantigen, an insulinoma antigen- 2, an Islet cell antigen, a thyroid stimulating hormone (TSH) receptor, a thyrotropin receptor, an Aggrecan, a CD4+T cell epitope, a porin, or an acetylcholine receptor.

In some embodiments, the rAAV vector is used to treat an autoimmune disease. In some embodiments, the autoimmune disease is selected from multiple sclerosis, disseminated sclerosis, encephalomyelitis disseminata, optic neuritis, celiac disease, or an allergic disease. In some embodiments, the autoimmune disease is multiple sclerosis.

In some embodiments, the present disclosure provides rAAV-based expression constructs that encode one or more mammalian therapeutic agent(s) (including, but not limited to, for example, protein(s), polypeptide(s), peptide(s), enzyme(s), antibodies, antigen binding fragments, as well as variants, and/or active fragments thereof), for use in the treatment, prophylaxis, and/or amelioration of one or more symptoms of a mammalian disease, dysfunction, injury, and/or disorder.

In some embodiments, the rAAV vector is of serotype AAV1, AAV3, AAV4, AAV5, AAV6, AAV7, AAV8, AAV9, AAV10, AAV11, AAV12, AAV2-AAV3 hybrid, AAVrh.10, AAVrh.74, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShHIO, AAV2(Y➜F), AAV8(Y733F), AAV2.15, AAV2.4, AAVM41, or AAVr3.45; or a derivative thereof, or any other serotype as known to one of ordinary skill in the viral arts. In some embodiments, the rAAV vector is of serotype AAV8. In some embodiments, the rAAV vector is pseudotyped.

In some aspects, the agent of the second composition is an immunosuppressive agent. In some embodiments, the agent of the second composition is an mTOR inhibitor. In some embodiments, the agent is rapamycin. In some embodiments of any one of the methods provided, the method further comprises administering an mTOR inhibitor, e.g., rapamycin. In some embodiments, the mTOR inhibitor is administered in a dose of 0.5 mg, 1 mg, 1.25 mg, 1.5 mg, 1.75 mg, 2 mg, 2.25 mg, 2.5 mg, 2.75 mg, 3 mg, 4 mg, 5 mg, or 6 mg per day. In some embodiments, the mTOR inhibitor is administered in a dose of 0.1 mg per day. In some embodiments, the agent is an oral mTOR inhibitor, such as an oral, FDA-approved mTOR inhibitor. In some embodiments, the mTOR inhibitor is a rapalog, e.g., temsirolimus (CCI-779), everolimus (RAD001), and ridaforolimus (AP-23573).

In some embodiments, the agent of the second composition is a sphingosine analog. In some embodiments, the agent is a sphingosine analog comprising fingolimod (e.g., Gilenya®). In some embodiments, the sphingosine analog is administered in a dose of 0.025 mg or 0.05 mg per day. In some embodiments, the agent is an oral sphingosine analog, such as an oral, FDA-approved sphingosine analog. In some embodiments, the agent is a sphingosine-1-phosphate receptor modulator or inhibitor. In some embodiments, the sphingosine-1-phosphate receptor modulator is fingolimod, ozanimod, or siponimod.

In some embodiments, the agent of the second composition is a monoclonal antibody. In some embodiments, the agent is natalizumab, alemtuzumab, or ocrelizumab. In some embodiments, the monoclonal antibody is administered in a dose of 300 mg every 28 days. In some embodiments, the agent is an intravenously administered biologic, such as an FDA-approved intravenously administered biologic.

In some embodiments, the agent of the second composition is a glucocorticoid. In some embodiments, the agent is a glucocorticoid comprising prednisone or prednisolone. In particular embodiments, the agent is prednisolone. In particular embodiments, the agent is prednisone. In some embodiments, the glucocorticoid is administered in a dose of 2.5 mg, 5 mg, 10 mg, 20 mg, 25 mg, or 50 mg daily. In some embodiments, the glucocorticoid is administered in a dose of 20 mg daily. In some embodiments, the glucocorticoid is administered in a dose of 20 mg per kg weight of the subject, daily. In some embodiments, the agent is an oral glucocorticoid, such as an oral, FDA-approved glucocorticoid. In some embodiments, the agent is prednisolone, and the agent administered in a dose of 5 mg, 10 mg, 15 mg, 20 mg, 25 mg, 30 mg, 35 mg, 40 mg, 45 mg, 50, mg, 55 mg, or 60 mg daily.

In some embodiments, the first composition is administered by intravenous injection. In some embodiments, the second composition is administered orally. In some embodiments, the second composition is administered by intravenous injection.

In some embodiments, the first composition and/or the second composition further comprises one or more pharmaceutically acceptable excipients.

The improved nucleic acid vectors and expression systems of the present invention may also optionally further include a polynucleotide that comprises, consists essentially of, or consists of, one or more polylinkers, restriction sites, and/or multiple cloning region(s) to facilitate insertion (cloning) of one or more selected genetic elements, genes of interest, or therapeutic or diagnostic constructs into the rAAV vector at a selected site within the vector.

In further aspects of the present invention, the exogenous polynucleotide(s) that may be delivered into suitable host cells by the rAAV nucleic acid vectors disclosed herein are of mammalian origin, with polynucleotides encoding one or more polypeptides or peptides of human, non-human primate, porcine, bovine, ovine, feline, canine, equine, epine, caprine, or lupine origin. In particular embodiments, the polynucleotides are of human origin.

The exogenous polynucleotide(s) that may be delivered into host cells by the disclosed viral nucleic acid vectors may, in certain embodiments, encode one or more proteins, one or more polypeptides, one or more peptides, one or more enzymes, or one or more antibodies (or antigen-binding fragments thereof), or alternatively, may express one or more siRNAs, ribozymes, antisense oligonucleotides, PNA molecules, or any combination thereof.

When combinational gene therapies are desired, two or more different molecules may be produced from a single rAAV expression system, or alternatively, a selected host cell may be transfected with two or more unique rAAV expression systems, each of which may comprise one or more distinct polynucleotides that encode a therapeutic agent. In some embodiments, a combination of two or more rAAV particles are administered to a mammalian subject to reverse or prevent proression of an autoimmune disease. In some embodiments, the mammalian subject is treated with any one of an rAAV.MOG, rAAV.PLP, rAAV.MBP, or a combination of two or three of these vectors. Such combination therapies, or cocktails, may comprise a composition comprising two or three of these vectors, or two or three compositions each comprising one of these vectors. In some embodiments, the serotype of the rAAV particles (capsids) of the combination therapy are the same (e.g., rAAV8). In some embodiments, the serotypes of the rAAV particles of the combination are different (e.g., rAAV8 and rAAV2).

In other embodiments, the present disclosure also provides rAAV nucleic acid vectors that are comprised within an infectious adeno-associated viral particle or a virion, as well as pluralities of such virions or infectious particles. Such vectors, particles, and virions may be comprised within one or more diluents, buffers, physiological solutions or pharmaceutical vehicles, or formulated for administration to a mammal (e.g., a human) in one or more diagnostic, therapeutic, and/or prophylactic regimens. The vectors, virus particles, virions, and pluralities thereof of the present invention may also be provided in excipient formulations that are acceptable for veterinary administration to selected livestock, exotics, domesticated animals, and companion animals (including pets and such like), as well as to non-human primates, zoological or otherwise captive specimens, and such like.

The present disclosure also concerns host cells that comprise at least one of the disclosed rAAV nucleic acid expression vectors, or one or more virus particles or virions that comprise such an expression vector. Such host cells are particularly mammalian host cells, such as human liver cells, and may be either isolated, in cell or tissue culture. In the case of genetically modified animal models, the transformed host cells may even be comprised within the body of a non-human animal itself.

Compositions comprising one or more of the disclosed rAAV nucleic acid vectors, expression systems, infectious rAAV particles, or host cells also form part of the present invention, and particularly those compositions that further comprise at least a first pharmaceutically-acceptable excipient for use in therapy, and for use in the manufacture of medicaments for the treatment of one or more mammalian inflammatory diseases, disorders, dysfunctions, or trauma. Such pharmaceutical compositions may optionally further comprise one or more diluents, buffers, liposomes, a lipid, a lipid complex. Alternatively, the rAAV nucleic acid vectors or rAAV particles of the present invention may be comprised within a plurality of microspheres, nanoparticles, liposomes, or any combination thereof.

Kits comprising one or more of the disclosed rAAV nucleic acid vectors (as well as one or more virions, viral particles, transformed host cells or pharmaceutical compositions comprising such vectors, virions, particle, or host cells); and instructions for using such kits in one or more therapeutic, diagnostic, and/or prophylactic clinical embodiments are also provided by the present invention. Such kits may further comprise one or more reagents, restriction enzymes, peptides, therapeutics, pharmaceutical compounds, or means for delivery of the composition(s) to host cells, or to an animal (e.g., syringes, injectables, and the like). Exemplary kits include those for treating, preventing, or ameliorating the symptoms of a disease, deficiency, dysfunction, and/or injury, or may include components for the large-scale production of the viral vectors themselves, such as for commercial sale, or for use by others, including e.g., virologists, medical professionals, and the like.

Another important aspect of the present invention concerns methods of using the disclosed rAAV nucleic acid vectors, virions, expression systems, compositions, and host cells described herein in the preparation of medicaments for diagnosing, preventing, treating or ameliorating at least one or more symptoms of a disease, a dysfunction, a disorder, an abnormal condition, a deficiency, injury, or trauma in an animal, and in particular, one or more autoimmune diseases in humans.

Compositions comprising one or more of the disclosed rAAV nucleic acid vectors, expression systems, infectious rAAV particles, and host cells also form part of the present invention, and particularly those compositions that further comprise at least a first pharmaceutically-acceptable excipient for use in the manufacture of medicaments and methods involving therapeutic administration of such rAAV nucleic vectors, rAAV particles, and host cells.

Another important aspect of the present invention concerns methods of use of the disclosed nucleic acid vectors, virions, expression systems, compositions, and host cells described herein in the preparation of medicaments for treating or ameliorating the symptoms of various autoimmune diseases, such as MS, in a mammal, and in particular one or more such diseases in a human.

Thus, some embodiments contemplate a method for preventing an autoimmune disease or inhibiting progression of the disease in a mammal (e.g., a human), the method comprising systemically administering to the mammal the first composition and the second composition in accordance with the methods disclosed herein in an amount and for a time sufficient to prevent or inhibit progression of the autoimmune disease in the mammal (e.g., a human). In some embodiments, the mammal has, is suspected of having, is at risk for developing, or has been diagnosed with the autoimmune disease. In some embodiments, the mammal is a newborn, an infant, a juvenile, an adult, or a young adult.

In some embodiments, the first composition is administered before the second composition, the first composition is administered after the second composition, or the first composition and the second composition are administered simultaneously. In some embodiments, the first composition and the second composition are administered simultaneously, e.g., in a single medical visit. In some embodiments, the first composition may be administered to a subject previously treated with an immunosuppressive agent. In some embodiments, an immunosuppressive agent may be administered to a subject that was previously treated with a composition comprising an rAAV vector or particle.

In some embodiments, the first composition and the second composition are admixed and administered as a single composition.

In some embodiments, the therapeutic molecule in the mammal reduces CNS inflammation, inhibits demyelination, re-establishes immune tolerance to one or more neuroproteins, stimulates the production of endogenous antigen-specific regulatory T cells, or any combination thereof. In some embodiments, the pharmaceutical composition comprising an effective amount of an rAAV vector of the disclosure is used as a medicament. In some embodiments, the rAAV vector is contemplated for use in treating or ameliorating one or more symptoms of multiple sclerosis in a mammal.

In some embodiments, the autoimmune disease is multiple sclerosis. In some embodiments, the progression of the autoimmune disease in the mammal is inhibited (e.g., the progression of one or more signs or symptoms of the disease is prevented) and/or reversed (e.g., one or more signs or symptoms of the disease is reversed) for at least 50 days, at least 75 days, at least 100 days, at least 125 days, at least 150 days, at least 175 days, at least 200 days, or more than 200 days after administration of the rAAV vector. In some embodiments, progression of the autoimmune disease in the mammal is inhibited and/or reversed for at least 150 days after administration of the rAAV vector. In some embodiments, the pharmaceutical composition comprising an effective amount of an rAAV vector of the disclosure is administered to the mammal in a single injection.

In some embodiments, expression of the therapeutic molecule in the mammal re-establishes immune tolerance to at least two different neuroproteins (e.g., after neuroprotein epitope spreading). In some embodiments, the at least two different neuroproteins comprise different epitopes of a single neuroprotein. In some embodiments, the single neuroprotein is a MOG protein. In some embodiments, the at least two different neuroproteins comprise at least one epitope of a MOG protein and at least one epitope of a PLP protein. In some embodiments, the at least two different neuroproteins comprise at least one epitope of a MOG protein and at least one epitope of an MBP protein.

In some embodiments, wherein the first nucleic acid encodes a full-length human MOG operably linked to a hepatocyte-specific promoter, further wherein the rAAV vector is of serotype AAV8.

In some embodiments, the rAAV nucleic acid vector is encapsidated by a rAAV particle as described herein. The rAAV particle may be of any AAV serotype (e.g., 1, 2, 3, 4, 5, 6, 7, 8, 9, or 10), including any derivative (including non-naturally occurring variants of a serotype) or pseudotype. In some embodiments, the rAAV particle is an AAV8 particle, which may be pseudotyped with AAV2 ITRs. Non-limiting examples of derivatives and pseudotypes include AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y➜F), AAV8 (Y733F), AAV2.15, AAV2.4, AAVM41, and AAVr3.45. In some embodiments, the rAAV vector is of serotype AAV8. In some embodiments, the rAAV vector is not of serotype AAV8. In some embodiments, the rAAV vector is pseudotyped. Such AAV serotypes and derivatives/pseudotypes, and methods of producing such derivatives/pseudotypes are known in the art (see, e.g., Mol Ther. 2012 Apr;20(4):699-708. doi: 10.1038/mt.2011.287. Epub 2012 Jan 24. The AAV vector toolkit: poised at the clinical crossroads. Asokan AI, Schaffer DV, Samulski RJ.). In some embodiments, the rAAV particle is a pseudotyped rAAV particle, which comprises (a) a nucleic acid vector comprising ITRs from one serotype (e.g., AAV2) and (b) a capsid comprised of capsid proteins derived from another serotype (e.g., AAV1, AAV3, AAV4, AAV5, AAV6, AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Exemplary rAAV nucleic acid vectors useful according to the disclosure include single-stranded (ss) or self-complementary (sc) AAV nucleic acid vectors, such as single-stranded or self-complementary recombinant viral genomes.

### Manufacture of rAAV particles

Methods of producing rAAV particles and nucleic acid vectors are also known in the art and commercially available (see, e.g., Zolotukhin et al. Production and purification of serotype 1, 2, and 5 recombinant adeno-associated viral vectors. Methods 28 (2002) 158-167; and U.S. Pat. Publication Nos. US20070015238 and US20120322861, which are incorporated herein by reference; and plasmids and kits available from ATCC and Cell Biolabs, Inc.). For example, a plasmid containing the nucleic acid vector sequence may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified.

In some embodiments, the one or more helper plasmids includes a first helper plasmid comprising a rep gene and a cap gene and a second helper plasmid comprising a E1a gene, a E1b gene, a E4 gene, a E2a gene, and a VA gene. In some embodiments, the rep gene is a rep gene derived from AAV2 and the cap gene is derived from AAV2 and includes modifications to the gene in order to produce a modified capsid protein described herein. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, PA; Cellbiolabs, San Diego, CA; Agilent Technologies, Santa Clara, Ca; and Addgene, Cambridge, MA; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R.O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap open reading frames (ORFs) for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV particle production. Alternatively, in another example Sf9-based producer stable cell lines are infected with a single recombinant baculovirus containing the nucleic acid vector. As a further alternative, in another example HEK293 or BHK cell lines are infected with a herpes simplex virus (HSV) containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

As used herein, the terms “engineered” and “recombinant” cells are intended to refer to a cell into which an exogenous polynucleotide segment (such as DNA segment that leads to the transcription of a biologically active molecule) has been introduced. Therefore, engineered cells are distinguishable from naturally occurring cells, which do not contain a recombinantly introduced exogenous DNA segment. Engineered cells are, therefore, cells that comprise at least one or more heterologous polynucleotide segments introduced through the hand of man.

To express a therapeutic agent in accordance with the present invention one may prepare a tyrosine capsid-modified rAAV particle containing an expression vector that comprises a therapeutic agent-encoding nucleic acid segment under the control of one or more promoters. To bring a sequence “under the control of” a promoter, one positions the 5′ end of the transcription initiation site of the transcriptional reading frame generally between about 1 and about 50 nucleotides “downstream” of (i.e., 3′ of) the chosen promoter. The “upstream” promoter stimulates transcription of the DNA and promotes expression of the encoded polypeptide. This is the meaning of “recombinant expression” in this context. Exemplary recombinant nucleic acid vector constructs are those that comprise an rAAV nucleic acid vector that contains a therapeutic gene of interest operably linked to one or more promoters that is capable of expressing the gene in one or more selected mammalian cells. Such nucleic acid vectors are described in detail herein.

### Pharmaceutical Compositions and Methods of Treatment

The genetic constructs of the present invention may be prepared in a variety of compositions, and may also be formulated in appropriate pharmaceutical vehicles for administration to human or animal subjects. The rAAV molecules of the present invention and compositions comprising them provide new and useful therapeutics for the treatment, control, and amelioration of symptoms of a variety of disorders, diseases, injury, and/or dysfunctions of the mammalian nervous system, and in particular, in the treatment or amelioration of MS. In some embodiments, the rAAV vectors of the present invention are used to treat an autoimmune disease. In some embodiments, the autoimmune disease is selected from multiple sclerosis, disseminated sclerosis, encephalomyelitis disseminata, optic neuritis, celiac disease, and/or an allergic disease. In some embodiments, the autoimmune disease is multiple sclerosis (MS). Thus, some embodiments contemplate a method of treating a mammal in need thereof comprising systemically administering to the mammal a therapeutically-effective amount of an rAAV vector as disclosed herein.

Some embodiments contemplate a method for preventing an autoimmune disease or inhibiting progression of the disease in a mammal, the method comprising systemically administering to the mammal an rAAV vector as disclosed herein in an amount and for a time sufficient to prevent or inhibit progression of the autoimmune disease in the mammal. In some embodiments, the mammal has, is suspected of having, is at risk for developing, or has been diagnosed with the autoimmune disease. In some embodiments, the autoimmune disease is multiple sclerosis, disseminated sclerosis, encephalomyelitis disseminata, optic neuritis, celiac disease, or an allergic disease. In some embodiments, the mammal is a newborn, an infant, a juvenile, an adult, or a young adult. In some embodiments, the mammal is a human.

In some embodiments, the expression of the therapeutic molecule in the mammal reduces CNS inflammation, inhibits demyelination, re-establishes immune tolerance to one or more neuroproteins, stimulates the production of endogenous antigen-specific regulatory T cells, or any combination thereof. In some embodiments, expression of the therapeutic molecule in the mammal re-establishes immune tolerance to at least two different neuroproteins. In some embodiments, the at least two different neuroproteins comprise multiple different epitopes of a single neuroproteins. In some embodiments, the rAAV vector comprises a nucleic acid segment that encodes a full-length mammalian MOG operably linked to a hepatocyte-specific promoter, wherein the rAAV vector is of serotype AAV8. In some embodiments, the rAAV vector is used as a medicament. In some embodiments, the rAAV is contemplated for use in treating or ameliorating one or more symptoms of multiple sclerosis in a mammal.

In some embodiments, the autoimmune disease is multiple sclerosis. In some embodiments, the progression of the autoimmune disease in the mammal is inhibited (e.g., the progression of one or more signs or symptoms of the disease is prevented) or reversed (e.g., reverse one or more signs or symptoms of the disease) for at least 50 days, at least 75 days, at least 100 days, at least 125 days, at least 150 days, at least 175 days, at least 200 days, or more than 200 days after administration of the rAAV vector. In some embodiments, progression of the autoimmune disease is inhibited or reversed for at least 180 days, 1 year, 1.25 years, 1.75 years, 2 years, 3 years, 4 years, 5 years, or more than 5 years in a subject (e.g., a human subject) after administration. In particular embodiments, progression of the autoimmune disease in the mammal is inhibited or reversed for at least 2 years after administration of the rAAV vector. In some embodiments, the rAAV vector is administered to the mammal in a single injection.

In some embodiments, this disclosure contemplates using the disclosed vectors to treat pre-existing neurological symptoms (e.g., muscle weakness in humans, or complete tail paralysis in mouse subjects) via the reversal of such symptoms. In some embodiments, the pre-existing neurological symptoms (for example those symptoms associated with the condition comprising MS in humans, or EAE in mice) are induced. In some embodiments, the subject having pre-existing neurological symptoms is treated with an rAAV vector (e.g., one or more rAAV vectors encoding one or more MOG, PLP, and/or MBP proteins, for example AAV8-MOG) as described herein. In some embodiments, the subject treated with an rAAV vector of the disclosure does not exhibit a harmful cytotoxic T cell response.

In some embodiments the subject is a human. In some embodiments, the human has, and/or has been diagnosed as having, one or more diseases or conditions. In some embodiments, the human has one or more symptoms of a disease or condition. In some embodiments, the human has the disease or condition for any length of time (for example recently diagnosed, long term chronic disease, recurring disease, etc.). In some embodiments, the one or more diseases or conditions comprises MS. In some embodiments, the subject is a non-transgenic mouse expressing pre-existing neurological symptoms.

In some embodiments, the number of rAAV particles administered to a subject may be on the order ranging from 10⁶ to 10¹⁴ particles/ml or 10³ to 10¹⁵ particles/ml, or any values therebetween for either range, such as for example, about 10⁶, 10⁷, 10⁸, 10⁹, 10¹⁰, 10¹¹, 10¹², 10¹³, or 10¹⁴ particles/ml. In one embodiment, rAAV particles of higher than 10¹³ particles/ml may be administered. In some embodiments, the number of rAAV particles administered to a subject may be on the order ranging from 10⁶ to 10¹⁴ vector genomes(vgs)/ml or 10³ to 10¹⁵ vgs/ml, or any values therebetween, such as for example, about 10⁶, 10⁷, 10⁸, 10⁹, 10¹⁰, 10¹¹, 10¹², 10¹³, or 10¹⁴ vgs/ml. In one embodiment, rAAV particles of higher than 10¹³ vgs/ml are administered. The rAAV particles can be administered as a single dose or divided into two or more administrations as may be required to achieve therapy of the particular disease or disorder being treated. In some embodiments, 0.0001 ml to 10 mls, e.g., 0.001ml, 0.01ml, 0.1ml, 1 ml, 2ml, 5ml or 10 ml, are delivered to a subject. In some embodiments, the number of rAAV particles administered to a subject may be on the order ranging from 10⁶-10¹⁴ vgs/kg weight of the subject, or any values therebetween, such as for example, about 10⁶, 10⁷, 10⁸, 10⁹, 10¹⁰, 10¹¹, 10¹², 10¹³, or 10¹⁴ vgs/kg.

In some embodiments, the disclosure provides formulations of one or more viral-based compositions disclosed herein in pharmaceutically acceptable solutions for administration to a cell or an animal, either alone or in combination with one or more other modalities of therapy, and in particular, for therapy of human cells, tissues, and diseases affecting man.

In some aspects, the rAAV particle compositions described herein are administered in a combination therapy or method with other agents as well, such as, fingolimod. In other embodiments, agents such as proteins or polypeptides or various pharmaceutically-active agents, including one or more systemic or topical administrations of therapeutic polypeptides, biologically active fragments, or variants thereof, are co-administered with the disclosed rAAV particle compositions. The rAAV particles may thus be delivered along with various other agents as required in the particular instance. Such compositions may be purified from host cells or other biological sources, or alternatively may be chemically synthesized as described herein.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravitreal, intraocular, intravenous, intranasal, intra-articular, and intramuscular administration and formulation.

Typically, these formulations may contain at least about 0.1% of the therapeutic agent (e.g., rAAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver rAAV particles in suitably formulated pharmaceutical compositions disclosed herein either subcutaneously, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection. The pharmaceutical forms of the compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

In certain circumstances it will be desirable to deliver compositions comprising an agent to a subject orally. In particular embodiments, it will be desirable to administer these compositions in accordance with FDA-prescribed guidelines (e.g., if the agent is FDA-approved). In some embodiments, the compositions are administered in an oral dosage form in accordance with an FDA-approved label.

The term “carrier” refers to a diluent, adjuvant, excipient, or vehicle with which the rAAV particle is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Other exemplary carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose.

The compositions of the present disclosure can be administered to the subject being treated by standard routes including, but not limited to, pulmonary, intranasal, oral, inhalation, parenteral such as intravenous, topical, transdermal, intradermal, transmucosal, intraperitoneal, intramuscular, intracapsular, intraorbital, intravitreal, intracardiac, transtracheal, subcutaneous, subcuticular, intraarticular, subcapsular, subarachnoid, intraspinal, epidural and intrasternal injection. In some embodiments, the composition is administered intravenously, by hepatic artery infusion, portal vein injection, or intrasplenic injection. In some embodiments, the composition comprises a AAV8 rAAV particle comprising a rAAV nucleic acid vector as described herein, and the composition is administered intravenously.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington’s Pharmaceutical Sciences” 15th Edition, pages 1035-1038 and 1570-1580). Some variation in dosage may occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions may be prepared by incorporating the rAAV particles in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, exemplary methods of preparation are vacuum-drying and freeze-drying techniques which yield a powder of the active ingredient plus any additional desired ingredient from a previously sterile-filtered solution thereof.

The amount of rAAV particle compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of viral particles to provide therapeutic benefit to the patient undergoing such treatment. Alternatively, in some circumstances, it may be desirable to provide multiple, or successive administrations of the compositions, either over a relatively short, or a relatively prolonged period of time, as may be determined by the medical practitioner overseeing the administration of such compositions.

The composition may include rAAV particles or nucleic acid vectors either alone, or in combination with one or more additional active ingredients, which may be obtained from natural or recombinant sources or chemically synthesized.

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs), RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. Although any methods and compositions similar or equivalent to those described herein can be used in the practice or testing of the present invention, exemplary methods and compositions are described herein. For purposes of the present invention, the following terms are defined below:

The term “subject,” as used herein, describes an organism, including mammals such as primates, to which treatment with the compositions according to the present invention can be provided. Mammalian species that can benefit from the disclosed methods of treatment include, but are not limited to, humans; apes; chimpanzees; orangutans; monkeys; domesticated animals such as dogs and cats; livestock such as horses, cattle, pigs, sheep, goats, and chickens; and other animals such as mice, rats, guinea pigs, and hamsters. In some embodiments, the subject has, is suspected of having, is at risk for developing, or has been diagnosed with an autoimmune disease or disorder, such as multiple sclerosis, disseminated sclerosis, or encephalomyelitis disseminata. In some embodiments, the subject has an autoimmune disease or disorder, such as multiple sclerosis, disseminated sclerosis, or encephalomyelitis disseminata. Other exemplary autoimmune diseases include type 1 diabetes, Grave’s disease, arthritis (e.g., rheumatoid arthritis or PGIA), autoimmune uveitis, Peripheral Neuropathy, Myasthenia gravis, Lupus, and Crohn’s disease. In some embodiments, an autoimmune disease or disorder is associated with an infection (e.g., a microbial or viral infection).

The term “treatment” or any grammatical variation thereof (e.g., treat, treating, and treatment etc.), as used herein, includes but is not limited to, alleviating a symptom of a disease or condition; and/or reducing, suppressing, inhibiting, lessening, ameliorating or affecting the progression, severity, and/or scope of a disease or condition.

The term “effective amount,” as used herein, refers to an amount that is capable of treating or ameliorating a disease or condition or otherwise capable of producing an intended therapeutic effect.

The term “promoter,” as used herein refers to a region or regions of a nucleic acid sequence that regulates transcription.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The tern “vector,” as used herein, refers to a nucleic acid molecule (typically comprised of DNA) capable of replication in a host cell and/or to which another nucleic acid segment can be operatively linked so as to bring about replication of the attached segment. A plasmid, cosmid, or a virus are each exemplary vectors.

The term “substantially corresponds to,” “substantially homologous,” or “substantial identity,” as used herein, denote a characteristic of a nucleic acid or an amino acid sequence, wherein a selected nucleic acid or amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared.

The percentage of sequence identity may be calculated over the entire length of the sequences to be compared, or may be calculated by excluding small deletions or additions which total less than about 25 percent or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides.

When highly-homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988).

The term “operably linked,” as used herein, refers to that the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

In some embodiments, the subject having pre-existing neurological symptoms exhibits a mean clinical score of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 at the time of the treatment. In some embodiments, the subject having pre-existing neurological symptoms exhibits a mean clinical score of 0.3 or 0.8 at the time of injection.

In some embodiments, the subject having pre-existing neurological symptoms is treated for a period of time, for example 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, 121 days, 122 days, 123 days, 124 days, 125 days, 126 days, 127 days, 128 days, 129 days, 130 days, 131 days, 132 days, 133 days, 134 days, 135 days, 136 days, 137 days, 138 days, 139 days, 140 days, 141 days, 142 days, 143 days, 144 days, 145 days, 146 days, 147 days, 148 days, 149 days, 150 days, etc. In some embodiments, the subject having pre-existing neurological symptoms is treated every other day per day for a period of time. In some embodiments, the subject having pre-existing neurological symptoms is treated once per week for a period of time. In some embodiments, the subject having pre-existing neurological symptoms is treated once per day for a period of time. In some embodiments, the subject having pre-existing neurological symptoms is treated multiple times per day (for example 2, 3, 4, 5, etc. times per day) for a period of time.

In an exemplary embodiment, the subject having pre-existing neurological symptoms is treated one time with a composition comprising i) any of the disclosed rAAV vectors, and ii) any of the disclosed immunosuppressive agents. In some embodiments, the subject treated one time with any of the disclosed rAAV vectors does not exhibit a harmful cytotoxic T cell response. In some embodiments, the subject having pre-existing neurological symptoms that is treated one time with any of the disclosed rAAV vectors shows reversal of the pre-existing neurological symptoms. In some embodiments, the subject having pre-existing neurological symptoms that is treated one time with any of the disclosed rAAV vectors shows reversal of the pre-existing neurological symptoms for a period of time, for example 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, 121 days, 122 days, 123 days, 124 days, 125 days, 126 days, 127 days, 128 days, 129 days, 130 days, 131 days, 132 days, 133 days, 134 days, 135 days, 136 days, 137 days, 138 days, 139 days, 140 days, 141 days, 142 days, 143 days, 144 days, 145 days, 146 days, 147 days, 148 days, 149 days, 150 days, or longer, etc.

In some embodiments, the subject having pre-existing neurological symptoms that is treated one time with a pharmaceutical composition comprising an rAAV vector of the disclosure exhibits complete remission (e.g., the neurological symptoms never return) and regains lost function (e.g., in human subjects: muscle strength and/or complete use of musculature; in mouse subjects: use of hind legs and/or ability to freely ambulate). In some embodiments, all subjects having pre-existing neurological symptoms that are treated one time with a pharmaceutical composition comprising an rAAV vector of the disclosure, and responded to such treatment, regained the ability to freely ambulate.

Some embodiments also contemplate the re-challenge (e.g., a second attempt to induce a disease state) of the subjects who were pre-treated by e.g., pre-tolerization, as described elsewhere herein. These embodiments indicate the robustness of the treatment therapies disclosed herein. Thus, in some embodiments, the subject pre-treated by e.g., pre-tolerization via administration of a vector prior to disease onset (as described herein), and who is thus immunized via the vector treatment against the first attempt to induce disease, undergoes a second attempt to induce a disease state. In some embodiments, the induction of disease comprises administering antigenic peptides to the subject. In some embodiments, the antigenic peptides are EAE-inducing antigenic peptides.

### Pre-Tolerization, Pre-Treatment, and Re-Challenge of Subjects Using the Vectors and Pharmaceutical Compositions of the Disclosure

In some embodiments, the disclosure contemplates using the disclosed vectors to prevent disease by e.g. pre-tolerizing healthy subjects prior to disease onset. In some embodiments, the healthy subjects selected for preventative treatment by e.g. pre-tolerization are subjects with an established family history of the disease being treated. In some embodiments, the healthy subjects selected for preventative treatment by e.g. pre-tolerization are subjects who have tested positive for genetic or molecular markers known to be associated with the disease being treated.

In some embodiments, the subject selected for preventative treatment by e.g. pre-tolerization is administered any of the disclosed rAAV vector-containing particles or compositions prior to disease onset. In some embodiments, the subject treated with any of the disclosed rAAV vector compositions prior to disease onset does not exhibit a harmful cytotoxic T cell response. In some embodiments, the subject selected for preventative treatment by e.g. pre-tolerization is administered any of the disclosed rAAV vector compositions one time prior to disease onset. In some embodiments, the subject selected for preventative treatment by e.g. pre-tolerization is administered any of the disclosed rAAV vector compositions multiple times prior to disease onset (e.g., 1 time, 2 times, 3 times, 4 times, 5 times, 6 times, 7 times, 8 times, 9 times, 10 times, etc.). In some embodiments, any of the disclosed rAAV vector compositions is administered to a subject (e.g., a human subject) immediately before, simultaneously with, or immediately after administration of any of the disclosed compositions comprising an immunosuppressive agent. In particular embodiments, any of the disclosed rAAV vector compositions is administered at about the same time as any of the disclosed compositions comprising an immunosuppressive agent.

In some embodiments, the subject selected for preventative treatment by e.g. pre-tolerization who has been administered any of the disclosed rAAV vector-containing particles or compositions shows no symptoms (for example genetic, molecular, phenotypic, or any other symptoms) of disease for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, 121 days, 122 days, 123 days, 124 days, 125 days, 126 days, 127 days, 128 days, 129 days, 130 days, 131 days, 132 days, 133 days, 134 days, 135 days, 136 days, 137 days, 138 days, 139 days, 140 days, 141 days, 142 days, 143 days, 144 days, 145 days, 146 days, 147 days, 148 days, 149 days, 150 days, 151 days, 152 days, 153 days, 154 days, 155 days, 156 days, 157 days, 158 days, 159 days, 160 days, 161 days, 162 days, 163 days, 164 days, 165 days, 166 days, 167 days, 168 days, 169 days, 170 days, 171 days, 172 days, 173 days, 174 days, 175 days, 176 days, 177 days, 178 days, 179 days, 180 days, 181 days, 182 days, 183 days, 184 days, 185 days, 186 days, 187 days, 188 days, 189 days, 190 days, 191 days, 192 days, 193 days, 194 days, 195 days, 196 days, 197 days, 198 days, 199 days, 200 days, 201 days, 202 days, 203 days, 204 days, 205 days, 206 days, 207 days, 208 days, 209 days, 210 days, etc. following treatment.

In some embodiments, a state of disease is induced (for example EAE) in those subjects receiving preventative treatment by e.g. pre-tolerization using the vectors disclosed herein for the purpose of e.g. evaluating vector pre-treatment efficacy. In some embodiments, a subject is pre-treated by e.g. pre-tolerization via a single administration of rAAV vector-containing particle or composition before disease (for example EAE) is induced, for example 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, 121 days, 122 days, 123 days, 124 days, 125 days, 126 days, 127 days, 128 days, 129 days, 130 days, 131 days, 132 days, 133 days, 134 days, 135 days, 136 days, 137 days, 138 days, 139 days, 140 days, 141 days, 142 days, 143 days, 144 days, 145 days, 146 days, 147 days, 148 days, 149 days, 150 days, 151 days, 152 days, 153 days, 154 days, 155 days, 156 days, 157 days, 158 days, 159 days, 160 days, 161 days, 162 days, 163 days, 164 days, 165 days, 166 days, 167 days, 168 days, 169 days, 170 days, 171 days, 172 days, 173 days, 174 days, 175 days, 176 days, 177 days, 178 days, 179 days, 180 days, 181 days, 182 days, 183 days, 184 days, 185 days, 186 days, 187 days, 188 days, 189 days, 190 days, 191 days, 192 days, 193 days, 194 days, 195 days, 196 days, 197 days, 198 days, 199 days, 200 days, 201 days, 202 days, 203 days, 204 days, 205 days, 206 days, 207 days, 208 days, 209 days, 210 days, etc. before disease is induced. In specific embodiments, a subject is pre-treated by e.g. pre-tolerization via a single administration of AAV8-MOG 200 days before disease (for example EAE) is induced.

In some embodiments, the subject pre-treated with AAV8-MOG does not exhibit a harmful cytotoxic T cell response. In some embodiments, the pre-treatment results in the complete prevention of disease onset (e.g., EAE). In some embodiments, the pre-treatment results in the complete prevention of disease onset (e.g., EAE) for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, 121 days, 122 days, 123 days, 124 days, 125 days, 126 days, 127 days, 128 days, 129 days, 130 days, 131 days, 132 days, 133 days, 134 days, 135 days, 136 days, 137 days, 138 days, 139 days, 140 days, 141 days, 142 days, 143 days, 144 days, 145 days, 146 days, 147 days, 148 days, 149 days, 150 days, 151 days, 152 days, 153 days, 154 days, 155 days, 156 days, 157 days, 158 days, 159 days, 160 days, 161 days, 162 days, 163 days, 164 days, 165 days, 166 days, 167 days, 168 days, 169 days, 170 days, 171 days, 172 days, 173 days, 174 days, 175 days, 176 days, 177 days, 178 days, 179 days, 180 days, 181 days, 182 days, 183 days, 184 days, 185 days, 186 days, 187 days, 188 days, 189 days, 190 days, 191 days, 192 days, 193 days, 194 days, 195 days, 196 days, 197 days, 198 days, 199 days, 200 days, 201 days, 202 days, 203 days, 204 days, 205 days, 206 days, 207 days, 208 days, 209 days, 210 days, etc. following the attempted induction of disease in the subject. In some embodiments, the pre-treatment results in the complete prevention of disease onset (e.g., EAE) for 30 days following the attempted induction of disease in the subject. In some embodiments, the pre-treatment results in the complete prevention of disease onset (e.g., EAE) for 75-120 days following the attempted induction of disease in the subject.

Some embodiments also contemplate the re-challenge (e.g., a second attempt to induce a disease state) of the subjects who were pre-treated by e.g. pre-tolerization. These embodiments seek to understand the robustness of the treatment therapies disclosed herein. Thus, in some embodiments, the subject pre-treated by e.g. pre-tolerization via administration of a vector as described herein prior to disease onset who is immunized via the vector treatment against the first attempt to induce disease undergoes a second attempt to induce a disease state. In some embodiments, the induction of disease comprises administering antigenic peptides to the subject. In some embodiments, the antigenic peptides and EAE-inducing antigenic peptides.

In some embodiments, the second attempt at inducing disease onset occurs after the first attempt at inducing disease, for example 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, 121 days, 122 days, 123 days, 124 days, 125 days, 126 days, 127 days, 128 days, 129 days, 130 days, 131 days, 132 days, 133 days, 134 days, 135 days, 136 days, 137 days, 138 days, 139 days, 140 days, 141 days, 142 days, 143 days, 144 days, 145 days, 146 days, 147 days, 148 days, 149 days, 150 days after the first attempt.

In some embodiments, the pre-treatment by e.g. pre-tolerization using the vector(s) of the instant disclosure results in the complete prevention (e.g., 100% of subjects pre-treated with the vector do not experience symptoms of disease) of disease onset (e.g., EAE). In some embodiments, pre-treatment by e.g. pre-tolerization is administered 200 days before the first attempted induction of disease (e.g., EAE) in the subject. In some embodiments, the pre-treatment of the subject results in complete prevention of disease for 75-120 days, for example 100 days, following the first attempted induction of disease in the subject. In some embodiments, the pre-treatment of the subject results in complete prevention of disease for 75-120 days, for example 100 days, following the first attempted induction of disease in the subject, even after a second attempt (“re-challenge”) to induce disease (e.g., the administration of an MS or EAE inducer) is conducted, in some embodiments. In some embodiments, the second attempt to induce disease occurs 84 days after the first attempt.

In some embodiments, subjects pre-treated by e.g. pre-tolerization survive following the attempted onset of disease. In some embodiments, 10%, 20%, 30%, 40%, 50%, 55%, 60%, 65%, 70% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of subjects pre-treated by e.g. pre-tolerization survive following the attempted onset of disease. In an exemplary embodiment, 100% of subjects receiving the preventative treatment survive for at least 150 days (e.g., for 150 days or longer) following the attempted onset of disease.

In some embodiments, the re-administration of the vector induces a full primary immune response in the subject. In some embodiments, the re-administration of the vector induces a recall response in the subject. In an exemplary embodiment, a subject is co-administered AAV8-MOG with an immunosuppressant (e.g., fingolimod) prior to disease onset, disease (e.g., EAE) is induced, and AAV8-MOG is re-administered to the same subject 84 days after disease onset, inducing a full primary immune response. In some embodiments, the subject administered and re-administered AAV8-MOG does not exhibit a harmful cytotoxic T cell response.

Thus, in some embodiments, the current disclosure contemplates the use of the vector(s) as disclosed herein to induce the stable expression of an epitope, which in turn induces the in vivo production of antigen-specific Tregs both prior to, and for a period of time (e.g., over 100 days) following, disease onset (for example, the induction of the EAE condition in a mouse subject). In some embodiments, the antigen-specific Tregs are MOG-specific Tregs. In some embodiments, the amount of anti-specific Tregs in a sample is measured using e.g. an assay. In some embodiments, the assay is an antigen-specific MHC tetramer flow cytometry assay. In some embodiments, the level of antigen-specific Tregs present after vector administration is increased relative to the level of the antigen-specific Tregs present prior to vector administration.

In some embodiments, the first and/or second nucleic acid segments is operably controlled by a promoter to drive its expression. In some embodiments, the promoter is a promoter that drives expression of the nucleic acid segment in the liver of the subject, e.g., a mammalian subject. In some embodiments, the promoter comprises a mammalian cell-specific or a mammalian tissue-specific promoter. In some embodiments, the promoter comprises a hepatocyte-specific promoter.

In some embodiments, the hepatocyte-specific promoter promoter comprises human apolipoprotein E (hapoE). In some embodiments, the hepatocyte-specific promoter comprises a hepatic combinatorial bundle (HCB) promoter. In other embodiments, the hepatocyte-specific promoter comprises an albumin promoter, a human α1-antitrypsin promoter, a transthyretin (TTR) promoter, or an apolipoprotein E (apoE) promoter.

In some embodiments, the vector is co-administered with an agent that induces immunosuppression. In some embodiments, the induced immunosuppression is transient. In some embodiments, the agent that induces immunosuppression is an mTOR inhibitor. In some embodiments, the mTOR inhibitor is rapamycin.

In sum, the present disclosures demonstrate sustained, antigen-specific disease prevention and reversal in an autoimmune disease after a single injection of the claimed vector. Thus, the embodiments of the instant invention show that the claimed vector is capable of complete prevention and strong therapeutic reversal of EAE, a mouse model of multiple sclerosis.

### Nucleic Acids, Proteins, and Variants Thereof

The genetic constructs of the present invention may be comprised within an appropriate viral vector, e.g., an rAAV vector. The embodiments of the present disclosure provide for the targeted delivery of certain nucleic acid sequences using viral vector delivery for the treatment of disease. In some embodiments, the nucleic acid sequences encode a therapeutic molecule. In some embodiments the therapeutic molecule comprises a protein. In some embodiments, the therapeutic molecule comprises one of a myelin oligodendrocyte glycoprotein (MOG), a proteolipid protein (PLP), and a myelin basic protein (MBP). In some embodiments, the therapeutic molecule encodes one or more transcript variants of MOG, MBP, and/or PLP.

Some embodiments therefore contemplate the targeted delivery of a nucleic acid segment (or sequence) encoding a MOG protein using viral vector delivery for the treatment of disease. MOGs are myelin proteins of the immunoglobulin superfamily that are expressed at the outermost surface of myelin sheaths and oligodendrocyte membranes, thus making MOGs a potential target of cellular and humoral immune responses in inflammatory demyelinating diseases such as multiple sclerosis (MS). In some embodiments, the nucleic acid sequence encodes a wild-type MOG protein, or a functional fragment thereof. In some embodiments, the nucleic acid sequence encoding the wild-type MOG protein, or a functional fragment thereof, is SEQ ID NO: 2 or 15.

In some embodiments, the pharmaceutical compositions and methods of treatment of the disclosure do not comprise a single rAAV particle agent, but rather comprise of both an rAAV agent and an immunosuppressive agent (e.g., an mTOR inhibitor, a sphingosine analog, a targeted biologic, or a glucocorticoid).

In some embodiments, the rAAV particles of the disclosure do not comprise an AAV8 capsid. In some embodiments, the rAAV particles of the disclosure do not comprise a capsid selected from AAVrh.10 or AAVrh.74. In some embodiments, an AAV particle of the disclosure does not comprise a capsid selected from AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShHIO, AAV2(Y➔F), AAV8(Y733F), AAV2.15, AAV2.4, AAVM41, or AAVr3.45.

In various embodiments, any of the rAAV particles, compositions, and methods of treatment of the disclosure are intended for use in treatment of multiple sclerosis. In some embodiments, any of the rAAV particles, compositions, and methods of treatment are intended for use in treatment of a disease other than multiple sclerosis. In some embodiments, any of the disclosed particles and compositions are intended for use in treatment of disseminated sclerosis, an encephalomyelitis, or an allergic disease.

In some embodiments, any of the rAAV particles, compositions, and methods of treatment of the disclosure induce tolerization in a manner that bypasses T helper cells. In some embodiments, any of the rAAV particles, compositions, and methods of treatment of the disclosure induce tolerization in a manner that raises the amount or activity of Treg cells. In other embodiments, any of the rAAV particles, compositions, and methods of treatment of the disclosure induce tolerization in a manner that bypasses activity of Treg cells.

In some embodiments, any of the rAAV vectors, and compositions thereof, of the disclosure do not contain a polynucleotide (e.g., a cDNA sequence) that has been codon-optimized for human expression. For instance, in some embodiments, the rAAV vectors do not comprise a cDNA sequence encoding a MOG, MBP, or PLP peptide that was codon-optimized for human expression.

In some embodiments of the methods of treatment provided herein, the methods do not comprise the administration of an mTOR inhibitor agent. In some embodiments, the disclosed methods do not comprise the administration of rapamycin.

### EXAMPLES

The following examples are included to demonstrate exemplary embodiments of the present disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1-Re-Establishing Immune Tolerance to Neuroantigens by AAV Gene Therapy

The inventor has demonstrated that hepatocyte-restricted expression of an AAV-delivered neuroantigen establishes persistent immunological tolerance mediated by antigen-specific Tregs capable of preventing and reversing EAE in mice. This example describes the development of a protocol that persistently induces Tregs in vivo and prevents disease development in a murine model of MS. The example also determines if tolerance can induce remission of pre-existing EAE disease and substantially reduce clinical and tissue-associated pathology.

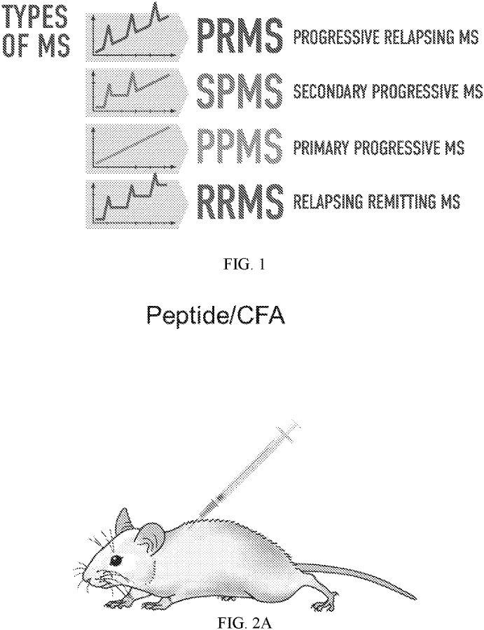

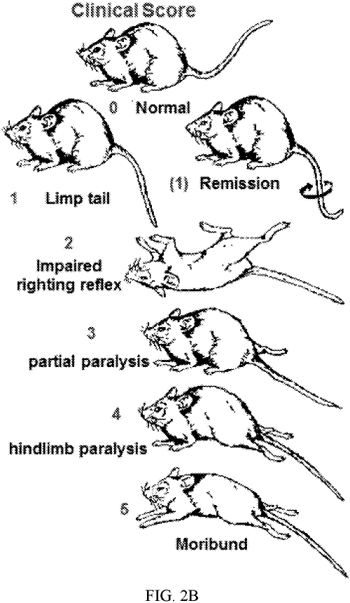

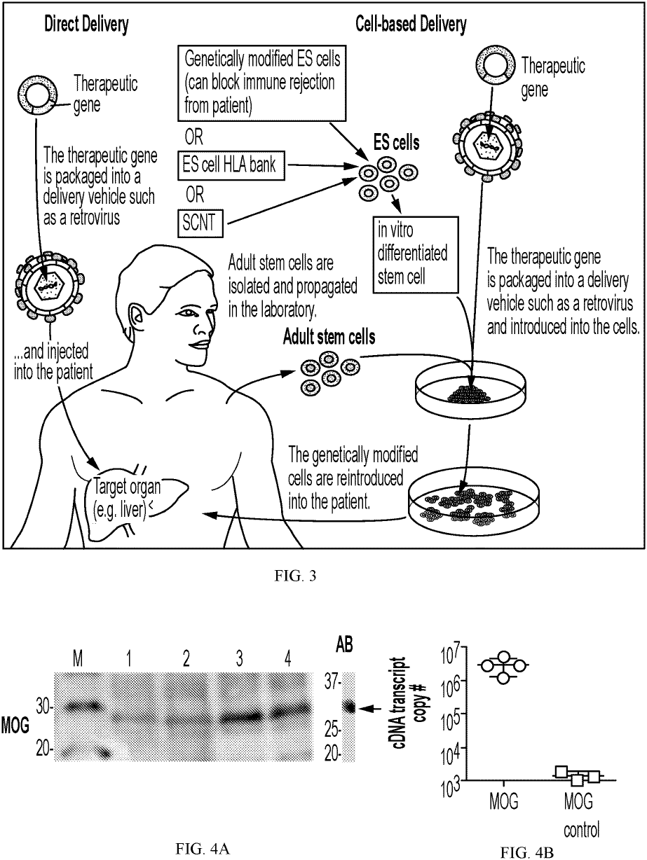

Neurodegenerative disease such as Multiple sclerosis (MS) is characterized by chronic infiltration of the CNS by pathogenic autoreactive lymphocytes that recognize neuroantigens. Functional defects in the endogenous regulatory T cells (Tregs) leading to a failure of central and/or peripheral mechanisms required for maintaining immunological tolerance combined with T cells recognizing myelin protein peptides are implicated in the pathogenesis of the disease. In C57BL/6 mice, experimental autoimmune encephalomyelitis (EAE) induced by myelin oligodendrocyte glycoprotein (MOG) produces a CD4 T cell-mediated inflammatory CNS disease that serves as a relevant model for MS (FIGS. 1, 2A and 2B).

Hepatic gene transfer with AAV vectors containing liver specific promoters can produce stable transgene expression and induce a robust antigen-specific immune tolerance to a variety of therapeutic proteins. It has been reported that induced Tregs not only suppress cellular immune responses against the transgene product but can also suppress humoral responses. Importantly, it has been shown that immune tolerance established by antigen expression in the liver is maintained even when the antigen was subsequently expressed in a highly immunogenic manner in other organs, such as skeletal muscle or intravenously.

The development of protocols that stimulate an increase in Treg numbers and/or their function has become a focus in treating autoimmune disease. Many of the beneficial effects of currently approved immunomodulators used in the treatment of MS are associated with restoring Treg homeostasis. This example demonstrates that liver-directed AAV gene therapy represents a novel approach to halt disease progression by restoring normal Treg function at disease onset.

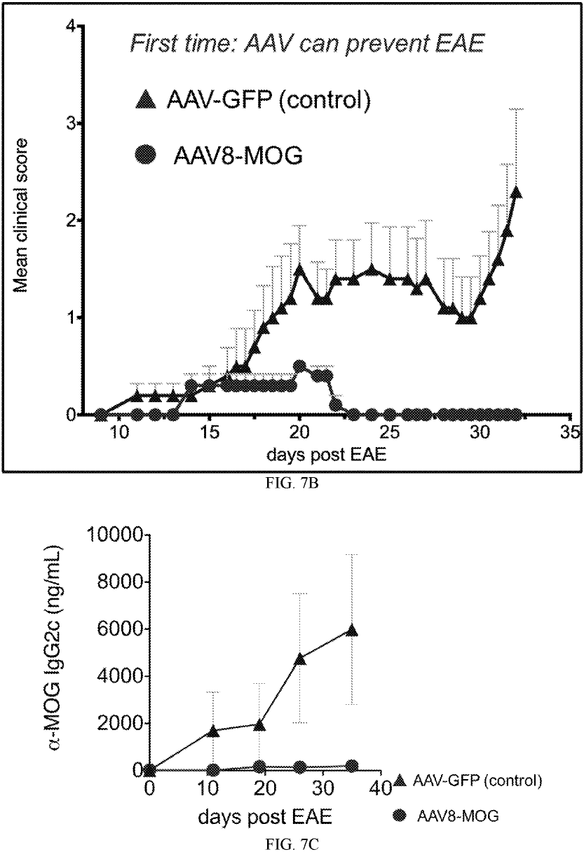

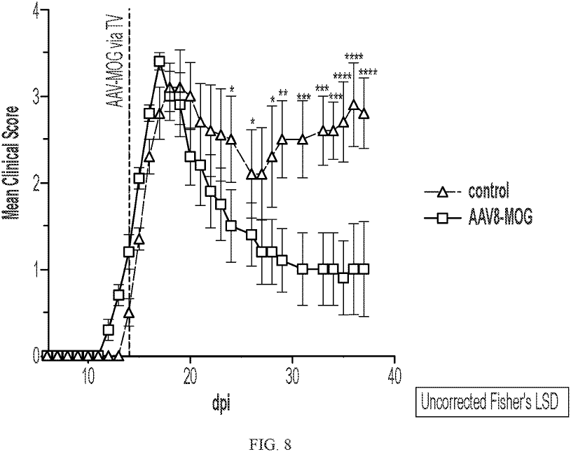

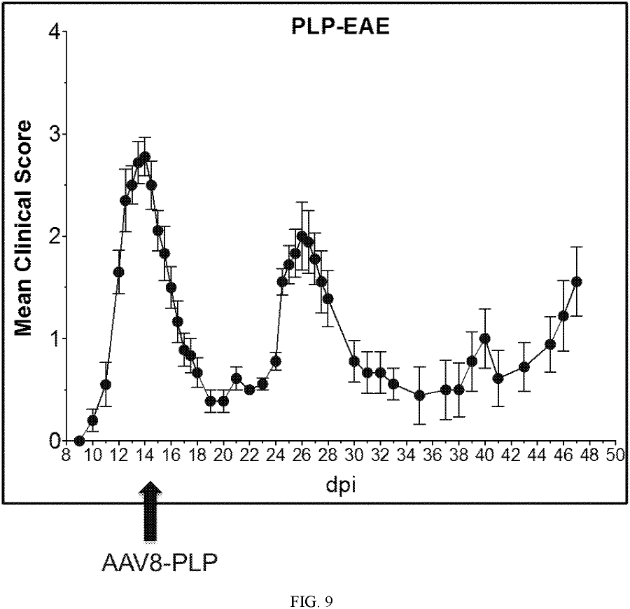

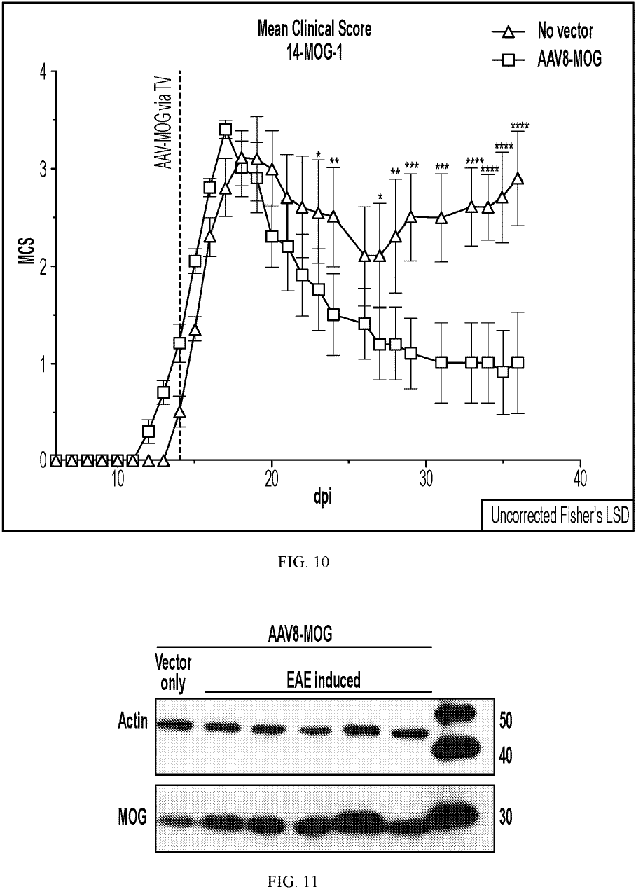

First, an AAV8-MOG vector was generated, and hepatic expression of the transgene in mice was validated by western blot and qPCR analysis (FIGS. 4). Next, to determine if hepatic expression of MOG can provide protection against the development of EAE mice were injected with either AAV8-MOG or -GFP vector. 2 weeks later EAE was induced and the mice were monitored and scored according to the classic scale for clinical signs of EAE. Plasma was obtained at 0-, 7-, and 14-days post EAE or at 0, 11-, 19-, 26-, and 35-days post EAE. The results revealed that mice receiving AAV8-MOG were clearly protected from developing EAE. Furthermore, these mice also did not produce any anti-MOG IgG1 or IgG2c autoantibodies. In contrast, those mice receiving the control vector developed severe EAE with elevated antibody titers (FIGS. 7 and 8).

### Example 2 - Therapeutic Molecules for AAV-Based Gene Therapy of MS

This example shows that liver directed gene transfer using an AAV vector expressing a neuro-antigen is capable of suppressing inflammation in the CNS and preventing EAE. Importantly, using AAV to express a full-length neuropeptide (or myelin-associated peptide) will enable greater applicability across MS-associated HLA haplotypes. Ongoing plans are to evaluate reversal of pre-existing EAE and functional analysis of the interplay of effector (Th1/Th17) cells and Tregs.

Using the following sequences for full length proteins, HLA/MHC restrictions were avoided.

MBP sequence in vector:

MGNHSGKRELSAEKASKDGEIHRGEAGKKRSVGKLSQTASEDSDVFGEAD
AIQNNGTSAEDTAVTDSKHTADPKNNWQGAHPADPGNRPHLIRLFSRDAP
GREDNTFKDRPSESDELQTIQEDPTAASGGLDVMASQKRPSQRSKYLATA
STMDHARHGFLPRHRDTGILDSIGRFFSGDRGAPKRGSGKVSSEP* (SE
Q ID NO: 1)

PLP sequence in vector:

MGLLECCARCLVGAPFASLVATGLCFFGVALFCGCGHEALTGTEKLIETY
FSKNYQDYEYLINVIHAFQYVIYGTASFFFLYGALLLAEGFYTTGAVRQI
FGDYKTTICGKGLSATVTGGQKGRGSRGQHQAHSLERVCHCLGKWLGHPD
KFVGITYALTVVWLLVFACSAVPVYIYFNTWTTCQSIAFPSKTSASIGSL
CADARMYGVLPWNAFPGKVCGSNLLSICKTAEFQMTFHLFIAAFVGAA (
SEQ ID NO: 2)

MOG sequence in vector:

MACLWSFSLPSCFLSLLLLLLLQLSCSYAGQFRVIGPGYPIRALVGDEAE
LPCRISPGKNATGMEVGWYRSPFSRVVHLYRNGKDQDAEQAPEYRGRTEL
LKETISEGKVTLRIQNVRFSDEGGYTCFFRDHSYQEEAAMELKVEDPFYW
VNPGVLTLIALVPTILLQVSVGLVFLFLQHRLRGKLRAEVENLHRTFDPH
FLRVPCWKITLFVIVPVLGPLVALIICYNWLHRRLAGQFLEELRNPL (S
EQ ID NO: 3)

EAE inducing peptide in SJL mice PLP₁₃₉₋₁₅₁: HCLGKWLGHPDKF (SEQ ID NO: 4).

EAE inducing peptide in C57BL mice: NTWTCQSIAFP (SEQ ID NO: 5) or PLP₁₇₈₋₁₉₁: NTWTTCQSIAFPSK (SEQ ID NO: 13).

C57BL: MOG₃₅₋₅₅: MEVGWYRSPFSRVVHLYRNGK (SEQ ID NO: 6).

SJL: MOG₉₂₋₁₀₆: DEGGYTCFFRDHSYQ (SEQ ID NO: 7).

### Example 3 - RAAV8 Vectors for Gene Therapy of MS

AAV8 vectors can stably express a neuro-protein in hepatocytes. AAV8-MOG can prevent the development of EAE, and AAV8-MOG can abrogate clinical symptoms of established EAE.

This example describes the development of a (pre)clinically relevant therapy using viral gene transfer that will result in the induction and expansion of antigen-specific T cells, re-establishing immunological tolerance as a treatment for multiple sclerosis. The approach has broad application as it uses full length myelin oligodendrocyte glycoprotein (MOG) protein and thus abrogates the need to identify HLA/MHC specific epitopes for inducing antigen specific Tregs. The knowledge gained from the work presented here will have the potential of creating a new line of treatment protocols for patients with MS as well as advance the research for both the MS and gene therapy fields.

Collectively, there is clear rationale for therapeutic approaches that are multifactorial. The present invention provides a novel therapy that not only focuses on reducing CD4⁺ T cells, but that can also target the effect of CD8⁺ T cells, B cells, and B cell derived components of the immune system.

### EXPERIMENTAL METHODS

Prevention of EAE: C57B1/6 mice will be injected with AAV8-MOG or control vector for hepatocyte-specific expression. At 4 weeks after gene transfer, induction of EAE will begin. Mice will be monitored daily and neurological impairment will be recorded on a detailed clinical scale. Weekly, blood/serum will be collected and analyzed for frequency of activated CD4, CD8, and Tregs by flow cytometric analysis, and α-MOG antibody formation will be quantified via ELISA. Upon termination, histology of harvested tissues will be evaluated for transgene expression (liver), magnitude and phenotype of infiltration of inflammatory cells, demyelination, and white matter damage (CNS). Establishing a baseline correlation of histology to clinical score (especially in control mice) will be essential for identifying success in subsequent aims.

Reversal of EAE: In this disclosure, induction of EAE in mice is first performed. Upon the first signs of EAE, mice will be randomly selected to receive hepatic gene transfer using AAV8-MOG or control vector. A detailed clinical assessment will be recorded daily. At various time points, blood/serum will be collected and analyzed as above. Upon sacrificing, liver and CNS tissue will be harvested and preserved for pathological and histochemical analysis.

Ex vivo functional analysis of tolerogenic Tregs: To determine if the AAV8-MOG induced Tg-specific Tregs are immunosuppressive, GFP⁺Tregs isolated by FACS from transgenic mice (“Foxp3^{EGFP}” B6.Cg-Foxp3^{tm2Tch}/J) that received vector 4 weeks earlier will be co-cultured with allogeneic splenocytes obtained from 2D2-TCR mice (MOG specific TCR) in the presence of MOG peptide. Cells and culture supernatant will be analyzed for activation, apoptosis, Th1/Th2/Th17 cytokines, or CTL activity via specific assays.

In vivo adoptive transfer of Tregs: To test whether the immunosuppressive function of Tg-specific Tregs is able to attenuate disease progression, GFP⁺Tregs, isolated as above, will be adoptively transferred into (a) naive mice that will subjected to EAE induction 24 hours later and (b) mice that have undergone MOG-induced EAE. At various time points, blood/serum will be collected, and liver and CNS tissue will be harvested and analyzed as above.

The overall theme of the present invention is the development of a gene therapybased method for in vivo induction of endogenous antigen (Ag)-specific regulatory T-cells (Tregs) using liver-directed Adeno-associated virus (AAV) gene therapy, as a novel treatment strategy for autoimmune diseases, e.g., multiple sclerosis (MS).

As noted above, MS is an autoimmune neurodegenerative disease of the central nervous system (CNS) in which the etiology is not well understood. Although auto-aggressive CD4⁺ T cells play a central role, the breakdown of immune tolerance mechanisms that permit activation of naive myelin-specific T cells is considered an initial step in the pathogenesis of MS. A number of pivotal studies in rodent models have substantiated that Ag-specific Tregs have a significant role in modulating autoimmune CNS disease and can be highly effective at treating MS.¹⁻⁵ Consequentially, there has been a major focus in developing protocols that stimulate Treg numbers and their function. Unfortunately, successful therapeutic use of Tregs has been limited by the lack of safe and effective Ag-specific protocols for isolation and expansion that are suitable for translation.

Using the AAV gene transfer platform, it has been clearly demonstrated that hepatocyte-restricted transgene expression from an optimized AAV vector can reliably induce immune tolerance to various therapeutic proteins, including coagulation factor IX (F.IX), α-1-antitrypsin, erythropoietin, and lysosomal storage enzymes, among others.⁶ Tolerance induction after hepatic gene transfer involves a combination of mechanisms. Importantly, AAV induced tolerance is mediated by Ag-specific CD4⁺CD25⁺FoxP3⁺ Tregs, which is critically dependent on achieving and maintaining adequate hepatocyte-restricted transgene expression.⁷⁻⁹ It has also been demonstrated that AAV induced Tregs can actively suppress antibody formation and cytotoxic CD8⁺ T cell responses against the transgene product.^{7,} ^{10,} ¹¹ Tolerized animals fail to form antibodies to the transgene even after subsequent attempts to immunize with protein formulated in adjuvant.¹⁰⁻¹² Efficient hepatic gene transfer induces a TGF-β dependent CD4⁺CD25⁺FoxP3⁺ Treg response that confers a dominant state of Ag-specific immune tolerance that is maintained even when the antigen was later introduced in other tissues in a highly immunogenic manner.^{7,} ¹² Induction of programmed cell death of effector T cells further tilts the balance toward tolerance, which is effectively enforced by induced Treg.^{13,} ¹⁴ Published data have demonstrated that hepatic AAV tolerance can also reverse pre-existing immune responses to F.IX in a hemophilia B mouse model.¹⁵ These now well-established concepts have been further supported by results from other laboratories and have led to the development of several immune tolerance protocols for genetic diseases.¹⁶⁻³⁷

Over 400,000 people in the United States currently are living with MS, and 10,000 new cases are diagnosed each year. With a 1:600-800 lifetime risk of developing the disease, MS is the most common cause of neurologic disability in young adults between 18 and 45 years of age. This demographic represents the majority of the adult workforce in the United States; therefore, the direct and indirect costs of health care for this population currently are estimated at $12 billion annually.³⁸

Multiple sclerosis (MS) is a protracted, immune-mediated disease of the CNS. MS is a neuroinflammatory autoimmune disease in which T cell-driven inflammation leads to demyelination and damage of axons. Although the exact pathogenesis of MS remains unknown, it is believed that myelin-specific CD4⁺ T cells play a central role in initiating and orchestrating CNS inflammation. A failure of central and peripheral mechanisms (particularly Tregs) to maintain self-tolerance and control potentially pathogenic auto-reactive lymphocytes is thought to be a key event in the development and pathogenesis of MS.^{4, 39-41} Several studies using in vitro suppression assays have documented functional impairments of Tregs from MS patients.^{42, 43} Experiments in mice using adoptive transfer of myelin-specific Tregs or Treg depletion have also provided evidence that Tregs can control the development and severity of experimental autoimmune encephalomyelitis (EAE) and accumulate within the CNS during the recovery.⁴⁴ It has also been shown that transgenic mice expressing myelin basic protein (MBP) could prevent the onset of EAE disease in mice in a Treg dependent process.^{45,} ⁴⁶ In fact, the mechanism-of-action for several of the currently approved immune-modulators used in the treatment of MS are associated with restoring Treg homeostasis.^{39,} ^{47,} ⁴⁸

Cumulatively, the literature clearly supports the concept that Treg cells influence the susceptibility and progression of disease. Recent advances have led to the recognition that Ag-specific Tregs represent an ideal form of cell therapy for MS. However, Tregs are still among the least understood T cell subsets, and consequently the most difficult to use for therapeutic applications.

Gene therapy with AAV vectors induces antigen-specific immune tolerance. Gene therapy continues to be a proven and powerful new tool for the treatment of a broad spectrum of diseases.⁴⁹ AAV vectors specifically have had great successes with in vivo gene transfer to a variety of target tissues.¹² For example, AAV gene transfer to retinal epithelial cells restores vision in children with Leber Congenital Amaurosis (LCA) and with Choroideremia.^{50,} ⁵¹ An AAV vector for treatment of lipoprotein lipase is the first gene therapy drug approved in the Western world (“Glybera”).⁵² Gene therapy by hepatic AAV administration has resulted in sustained expression of factor IX (F.IX) at levels of >5% of normal in hemophilia B patients, changing their bleeding phenotype from severe to mild.⁴⁸ Hepatic AAV gene transfer promotes tolerance via induction of transgene product-specific Treg, a phenomenon that can be exploited for the treatment of MS.^{6,} ^{21,} ^{34,} ^{35,} ^{49,} ⁵³

Effective therapy for established EAE needs to consider induction of multiple direct and indirect (cross-tolerance) regulatory mechanisms, including the induction of antigen (Ag)-specific CD4⁺CD25⁺FoxP3⁺ Tregs across multiple endogenous myelin epitopes (epitope spreading). Neutralization of epitope spreading remains one of the most elusive aspects of developing effective MS treatments. See Zhang et al., J. Immunol., 184:6629-6636 (2010), herein incorporated by reference.

The literature supports the idea that Tregs are potent suppressors of EAE, and essential to establish disease remission. However, very few studies have addressed how to generate such Ag-specific Tregs in a manner that is both reliable and translatable. The present disclosure demonstrates that hepatic gene transfer using AAV8 vectors to express full-length myelin-associated proteins will induce Ag-specific Tregs across multiple endogenous epitopes in a manner that has been shown to be safe, feasible, and long lasting. This disclosure is innovative in several respects: (i) This is the first time a clinically proven AAV vector technique is used to re-establish immunological tolerance in the context of an autoimmune disease; (ii) exemplary AAV8 vectors have been designed to express a full-length neuro-protein (myelin oligodendrocyte glycoprotein (MOG) or proteolipid proteins (PLP)), thus abrogating the need for identifying HLA/MHC specific epitopes and enhancing the potential for success; and (iii) Based on published data, incorporating transient immune modulation using the FDA approved mTOR inhibitor rapamycin should provide a synergistic effect, facilitating tolerance induction to neuroantigens by further tipping the balance from Teff to Treg in vivo.⁵⁴⁻⁵⁶

The inventor has shown that AAV8 liver gene transfer of a neural protein (PLP or MOG) induces activation of Ag-specific Tregs, and is sufficient to re-establish immune tolerance and abrogate disease progression in the CNS of a murine model for MS.

Hepatic gene transfer with AAV vectors can reliably induce a robust antigen-specific immune tolerance in experimental animals to a variety of therapeutic proteins.^{7,} ^{9, 13,} ^{54,57} Here, tolerance was characterized by lack of antibody formation, helper T cell response, or CTL response to the transgene product, even after subsequent challenge with protein in adjuvant. Using mice transgenic for a T cell receptor, evidence of anergy and deletion of transgene product-specific CD4 T cells was found.

That immune tolerance established by hepatic transgene expression is maintained even when the antigen was subsequently expressed in a highly immunogenic manner in other organs, such as skeletal muscle, or even delivered intravenously.⁷ These results revealed that liver directed gene therapy could abrogate potential cytotoxic CD8 T cell responses, indicating that the range of immune tolerance extends beyond the level of antigen expression initially achieved by hepatic gene transfer.

Hepatic AAV gene transfer efficiently and rapidly reversed pre-existing high antibodies titers and provided long-term correction of haemostasis in a murine hemophilia B model. ^{15,} ⁵⁶ High levels of transgene protein suppressed memory B cells and increased Treg induction, indicating direct and indirect mechanisms of suppression of inhibitor formation. There is an increasing body of evidence that B cells and autoantibodies may play a pathogenic role in demyelinating disease. ^{58,} ⁵⁹

Immune tolerance induction by hepatic AAV gene transfer does not require protein to be secreted. Although hepatic expression is crucial for tolerance induction, secretion from hepatocytes for systemic delivery of the transgene product is not required. Expression of a cytoplasmic a neo-antigen in as few as 3% of the hepatocytes is sufficient to induce Tregs and provide long-term suppression of inflammatory responses.⁵⁷

### RESULTS

Successfully establishing multiple models of EAE induction: EAE is a widely accepted experimental mouse model of multiple sclerosis that is induced in susceptible animals by immunization with central nervous system antigens. EAE is an autoimmune disease that is mediated by CD4⁺ T helper 1 (T_{H}1) cells and interleukin-17 producing T_{H}17 cells that are reactive to components of the myelin sheath. The cells infiltrate the nervous parenchyma, release pro-inflammatory cytokines and chemokines, promote leukocyte infiltration and contribute to demyelination.

EAE can be induced in various strains of mice using different neuro-proteins emulsified in complete Freud’s adjuvant (CFA). Disease progression and pathology manifests differently with each combination. For example, EAE induced by MOG produces encephalitogenic T-cells and demyelinating autoantibodies in C57BL/6 mice. The resulting disease is a chronic-progressive disease characterized by axonal demyelination and white matter lesions in the spinal cord, and is generally considered to be a relevant model for human immune-mediated demyelinating disease.⁶⁰ EAE can also be induced in SJL (H-2s) mice using the major encephalitogenic PLP peptide (PLP₁₃₉₋₁₅₁). Here the disease is characterized by a relapsing-remitting course of paralysis, which allows assessment of the efficacy of various immune regulatory strategies in a re-occurring disease setting.

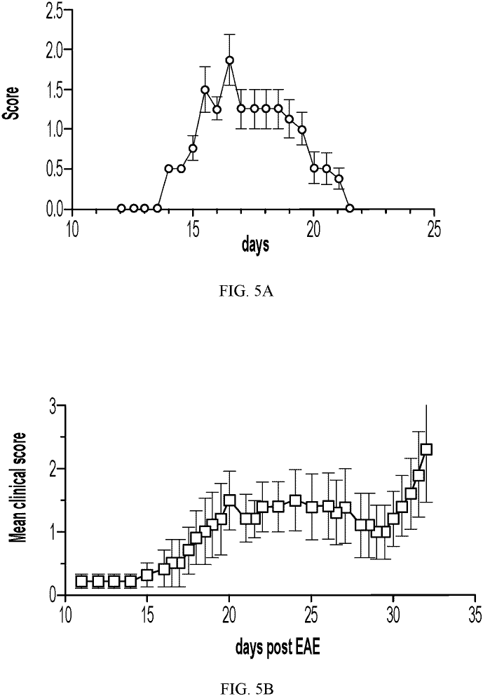

In this disclosure, the inventor demonstrates the timeline and clinical scoring for successful induction of EAE disease in two different mouse strains. In one experiment, 8-week-old female mice were injected subcutaneously with 200 µg myelin peptide emulsified in CFA containing 4 mg/ml Mycobacterium tuberculosis. Clinical signs of EAE began 12 days later at which time mice were evaluated twice daily. Mice were scored according to the severity of the clinical signs (FIG. 5A). In a similar experiment, EAE was induced in C57BL/6 mice (n = 5) using MOG in order to develop a chronic progressive EAE disease (FIG. 5B)

Novel AAV8 vectors transduce mouse hepatocytes efficiently and express the delivered neural protein: AAV is a non-pathogenic single stranded DNA parvovirus with a genome size of approximately 4.7 kb. Serotypes with distinct tissue tropisms have been isolated from multiple vertebrate species, including humans. Viral vectors derived from AAV are devoid of viral genes and instead contain an expression cassette for the gene of interest, which is limited to ~5 kb in length. In this disclosure, an AAV8 serotype vector was chosen because it has strong natural tropism for hepatocytes after peripheral vein administration, avoiding the need for an invasive procedure. Additionally, it fails to transduce professional antigen presenting cells (APCs). The engineered vector constructs include a strong and highly hepatocyte-specific promoter.¹⁰

The newly synthesized vectors were evaluated for transduction efficiency. To demonstrate efficacy, the inventor assessed whether mouse hepatocytes could be transduced and express the neuro-protein transgene following tail vein injection. A group of mice was injected with 1 × 10¹¹ vector particles of AAV8-ApoE/hAAT-MOG. Two weeks later, using liver lysates, evidence of hepatic expression of MOG was probed by both western blot and qPCR analysis. The results demonstrate the ability of this novel vector to stably produce hepatic expression of the neuro-antigen after liver gene transfer (FIG. 4A and FIG. 4B).

AAV8-MOG produces hepatic transgene expression that can prevent the establishment of EAE: Previously, others have shown that ectopic expression of a myelin-associated protein using various transient methodologies promoted resistance to EAE.^{18,} ^{28,} ^{45,} ⁶¹ Unfortunately, these prior approaches have not developed into practical therapies for human autoimmune disease. Prior to this invention, the ability of AAV liver gene transfer to induce antigen specific suppression of autoimmune disease went untested in the scientific community.

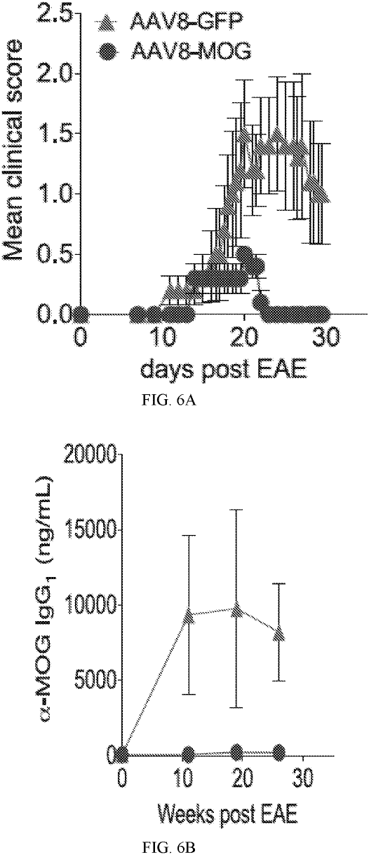

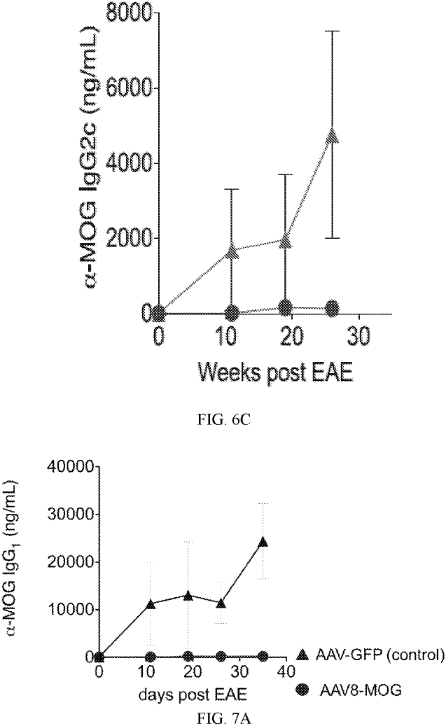

To further support this invention, a pilot study was performed. A small number of mice (n=5) were intravenously injected with 10¹¹ vector particles via the tail vein with either AAV8-MOG or AAV8-GFP (control) vector. Two weeks later, EAE was induced using MOG in CFA as previously performed. Plasma samples were obtained at 0-, 7- and 14-days post EAE induction or at 0, 11-, 19-, 26-, and 35-days post EAE induction. The mice that received AAV8-MOG were essentially protected from developing EAE (FIG. 6A, FIG. 6B, and FIG. 6C). In contrast, those mice receiving the control vector developed severe EAE with elevated antibody titers. This data indicates that the vectors described herein not only express in the liver, but also had an immune modulatory effect.

Active suppression by Tregs plays a key role in the control of auto-reactive T cells and the induction of peripheral tolerance in vivo. In particular, the significance of Ag-specific Tregs in conferring resistance to organ-specific autoimmunity and in limiting autoimmune tissue damage has been documented in many disease models, including MS.⁴⁴ However, a safe and clinically feasible method for sustained expansion of endogenous Tregs has yet been identified.^{41,} ^{44,} ^{60,} ⁶³ a treatment protocol based on liver-directed AAV gene therapy can durably induce Ag-specific tolerance, thus having the potential of blocking the pathogenic autoimmune response present in MS and inhibiting disease activity; while avoiding the severe side effects associated with many of the currently used immunotherapies. Based on these and related studies, AAV8-liver gene transfer can restore immunological tolerance against myelin-sheath antigens, such as MOG and PLP, by inducing Ag-specific Tregs in vivo.

Experimental approach and methods of analyses: This set of experiments tests vector constructs in order to verify efficiency of liver transduction and hepatic expression without adverse effects. Groups of (i) C57BL/6 or (ii) SJL/J mice (7-8 weeks old) will be injected with the 10¹¹ vector particles (vp) (effective dose of vector as previously determined) of (i) AAV8-MOG or (ii) AAV8-PLP (respectively), or control (irrelevant transgene, GFP) intravenously via the tail vein. Beginning on day 0, blood will be collected every 2 weeks and analyzed for the frequency of various T cell populations using standard markers of T cell phenotype (including, but not limited to, CD4, CD8, FoxP3, CD25, CD62L, CD44). Humoral immune responses (e.g., α-IgG1, -IgG2a, -IgG2c responses) may be determined via antigen specific ELISA. At 14 days post gene transfer, half of the mice from each group may be randomly selected and humanely euthanized. Tissues (blood, liver, spleen, and CNS (brain/spinal cord)) may be harvested for analysis. Hepatic transgene expression levels may be determined at the mRNA level using real-time quantitative PCR. Absolute and relative hepatic protein levels of the transgene will also be determined via western blot using liver lysates. At 90 days post injection, the remainder of the mice may be processed similarly to establish sustained transgene expression. Additionally, some mice may be subjected to EAE induction at various time points after vector administration and evaluated for prevention of disease, as described in preliminary data. Aliquots of the collected tissue samples may be archived as a reference material.

In vitro functional suppression analysis of Ag-specific Tregs induced by AAV8 hepatic gene transfer. Splenic Tregs (CD4⁺CD25⁺) may be magnetically sorted from mice that received (i) AAV8-MOG or (ii) AAV8-PLP or AAV8-GFP (control) vector and co-cultured with graded numbers of CFSE labeled cells obtained from 2D2-TCR mice (this C57B⅙ mouse line expresses a TCR which recognize MOG₃₅₋₅₅ in the context of H-2 IA^{b}) or splenocytes harvested and labeled from SJL mice that have been previously immunized with PLP/adjuvant in the presence of anti-CD3/CD28 coated beads (provides APC independent/non-specific activation of Teff). Treg mediated suppression of proliferating effector cells may be determined by flow cytometry. Cell-culture supernatants may be analyzed for Th1/Th2/Th17 cytokines via specific assays. Results may be compared with data from naive and EAE induced mice (in which many CD4⁺CD25⁺ cells should represent activated effector rather than Treg). This disclosure demonstrates Ag-specific functional suppression from the vector induced Tregs compared to controls.

Based on the initial data and published studies, maximal transgene expression may occur by 2 weeks, which remains fairly unchanged over time, thus indicating stable transduction of hepatocytes.¹⁰ Since the vector constructs have been purposely designed to express full length MOG or PLP and include a strong hepatocyte promoter, it is also expected that AAV8 vector-mediated expression may be constrained to the hepatocytes and not secreted. Sequestering the transgene will constrain pathological consequences of freely circulating AAV-derived neuroantigen. Lastly, the inventor does not expect inflammatory responses in any tissues and analysis of liver enzymes (ALT/AST) should demonstrate an absence of hepatotoxicity. Furthermore, based on pilot studies, it is expected that vector administration prior to EAE induction will prevent disease development. A positive outcome would also be the absence/significant reduction in antigen specific antibody responses. Lastly, results from the Treg suppression assays are expected to show that suppression induced by hepatic transgene expression is facilitated by activation of Ag-specific Tregs.

Even though the literature overwhelmingly supports the idea that Tregs are potent suppressors of EAE and are the driving force to switch from disease progression to remission, very few studies in the past have addressed a method by which to generate such Ag-specific Tregs that is both safe and effective.⁶⁴ In theory, this could be achieved by two approaches. The first would be to isolate Tregs, expand their numbers ex vivo, and then reintroduce them, with the idea that an increase in overall frequency of polyclonal Tregs might influence ongoing disease. In 2004, Bluestone’s group in a type-1 diabetes model provided initial proof of principle for this approach.⁶⁵ More recently, others have further shown that using expanded Tregs from myelin-specific transgenic TCR mice is more effective.⁶⁴ The second approach is to administer a suitable treatment that promotes the expansion of Treg numbers and/or function in vivo. Recent reports have described the use of various compounds (e.g., nano-particles/small molecules) to enhance Treg function in EAE, while others try to augment antigen presentation in order to generate Tregs.^{64,} ⁶⁶ In the end, a reliable and translatable method for induction of the disease relevant Ag-specific Tregs is still lacking— until now. This proposal presents a methodology that will provide a durable method for the continued in vivo induction of endogenous Ag-specific Tregs. Based on previous work, hepatic gene transfer using AAV8 vectors expressing full-length MOG or PLP should induce Ag-specific Tregs across multiple endogenous myelin epitopes in a manner that has been shown to be safe, feasible, and long-lasting.

Experimental approach and methods of analyses. Here, mice will first undergo active induction of EAE using either (i) MOG or (ii) PLP. At the first clinical signs of EAE, in MOG-induced chronic-progressive mice, or at the peak of disease, in PLP-induced relapsing-remitting mice, AAV8-MOG or AAV8-PLP vector (respectively) or AAV8-GFP for control mice may be given. Mice may be clinically scored by weight and neurological deficit 2x daily. Blood may be collected and analyzed for humoral (IgG) responses as before. At ~45 days, each cohort of mice may be perfused and randomly subdivided into 2 groups. Group 1 will have brain, spinal cord, and liver tissues harvested and preserved for histopathological and immunofluorescent analysis. Infiltrating lymphocytes may be isolated from the brain and spinal cords from mice in Group 2 (as previously described⁶⁷). The frequency of various T cell populations may be analyzed using standard markers of T cells (including, but not limited to, CD4, CD8, FoxP3, CD25, CD62L, CD44, CTLA-4, CD103). Liver tissue may be subjected to transcriptional and protein analysis as shown. Results may be compared to control mice and reference material. Portions of the tissue may also be archived for future studies.

It is expected that therapeutic treatment with a single injection of AAV8-MOG or -PLP vector at the onset or peak of the disease will result in a dramatic remission in clinical impairment. There should be a concurrent reduction in antibody titers and/or frequency of B cell responses to the EAE inducing peptide, as compared to control-vector treated mice. CNS inflammation is characteristic of EAE, and the degree of lymphocyte infiltration correlates with disease progression; whereas, the presence of Tregs in the CNS during EAE has been associated with diminished inflammation and resolution of clinical disease.⁶⁸ Hence, it is expected that a significant reduction of inflammatory infiltrates in the CNS of vector-treated mice will be observable upon histopathological analysis. This would suggest that the induced Ag-specific Tregs migrating to the site of CNS damage are protective and are capable limiting damage mediated by effector T cells. Additionally, the natural relapsing-remitting nature of PLP-induced EAE may be exploited by timing the injection of the AAV8-PLP vector so that the peak effects of induced tolerance correspond to when the disease is relatively quiescent (remitting).

On the other hand, mice that receive MOG for EAE induction begin showing neurological impairments after ~12 days, which progressively escalate. In this scenario, it is possible that some level of inflammation will still be present, although the phenotypic analysis of the T cell populations show that absolute numbers of T cells infiltrating the CNS is lower, with a greater Treg:Teff ratio.

Transient immunosuppression using rapamycin. Rapamycin readily crosses the BBB thus exerting direct effects within the CNS. Blocking the activation of the mTOR pathway, rapamycin prevents activation of T cells by inhibiting their response to IL-2 thus preventing Ag-induced proliferation of Teff, while selectively allowing expansion of functional CD4⁺CD25⁺FoxP3⁺ Tregs. In EAE, rapamycin is effective in preventing the onset of disease; however, suppression of established disease is only maintained with continued use.⁶⁹ In a further series of experiments, vector-treated mice are transiently immunosuppressed. Groups of mice are then injected with AAV8-MOG, -PLP, -GFP or PBS at specific time-points that correspond to either initial onset or peak of disease. Concurrently, mice receive intraperitoneal rapamycin (1 mg/kg), or PBS (sham control) daily for 14 consecutive days.⁶⁹ At specific time points corresponding to pre- and post-treatment and significant changes in clinical scoring, tissues and lymphocytes may be harvested from the CNS and spleen from randomly selected mice. Histopathological changes within the tissues can then be identified. Isolated cells are then phenotyped and the frequency of Tregs and Teffs from the different compartments may be determined and compared to control groups to validate the efficacy of rapamycin co-treatment.

Regardless of AAV8 administration, treatment with rapamycin alone is expected to transiently produce a rapid reduction in the clinical presentation of EAE because it selectively inhibits Teff proliferation.⁶⁹ However, when used in conjunction with AAV8 liver gene transfer, rapamycin treatment has a synergistic effect that results in an increase in vector induced Ag-specific FoxP3⁺ Tregs (since they are less sensitive to mTOR signaling inhibition) with a corresponding decrease in effector T cells.⁷⁰ The shift to tolerance is further potentiated by the fact Tregs have been shown to mediate selective inhibition of antigen-specific Th1 cells in the CNS of EAE.⁷¹

The data clearly supports the ability of AAV liver gene transfer to induce Ag-specific Tregs and invoke immune tolerance. Because accumulation of Tregs in the CNS during the recovery phase of EAE has been a consistent finding in actively induced models, it seems unlikely that the present therapy would not have, at least to some degree, a clinical or pathological benefit.^{3,} ^{64,} ^{71,} ⁷²

### Therapeutic Regimens

The therapeutic regimens presented herein address an unmet need by providing an effective treatment for diseases such as MS using a gene therapy approach. Using the AAV vector platform disclosed herein to deliver full-length proteins offers a superior HLA-independent approach for Ag-specific Treg induction compared to other ex vivo or epitope-restricted Treg mediated therapies. Additionally, AAV gene transfer results in continuous Treg generation because of the long-term hepatocyte expression of transgene.⁷³

In some embodiments, progression of an autoimmune disease (e.g., multiple sclerosis) in the mammal is inhibited or reversed for at least 50 days, at least 100 days, at least 150 days, at least 175 days, at least 200 days, or more than 200 days after administration of any of the disclosed rAAV particles or compositions comprising any of the disclosed rAAV nucleic acid vectors to the mammal. In particular embodiments, progression of the autoimmune disease in the mammal is inhibited or reversed for at least 125-150 days. In some embodiments, the mammal is an experimental animal, such as a rodent. In some embodiments, the mammal is a human.

In some embodiments, progression of an autoimmune disease (e.g., multiple sclerosis) in a mammal at risk of developing symptoms is prevented, either partially or completely. In some embodiments, progression is prevented for at least 50 days, at least 100 days, at least 150 days, at least 175 days, at least 200 days, or more than 200 days after administration of any of the disclosed rAAV particles or compositions comprising any of the disclosed rAAV nucleic acid vectors to the mammal.

In some embodiments, the composition or particle comprising the rAAV nucleic acid vector is administered to a mammal diagnosed with and/or suffering from an autoimmune disease such as multiple sclerosis (MS). In some embodiments, the mammal suffers from symptoms of the disease. In some embodiments, the mammal suffers from an early stage of the disease. In some embodiments, the mammal suffers from a late stage of the disease.

In some embodiments, the composition or particle comprising the rAAV nucleic acid vector is administered to the mammal in a single injection. In some embodiments, the particle is administered in two or more injections in a single doctor’s (physician) visit. In some embodiments, the particle is administered in two or more injections among multiple doctor’s visits, or throughout the course of a therapeutic regimen.

In some embodiments, the mammal is already receiving a course of AAV therapy at the time of administration of any of the compositions comprising an immunosuppressant (or DMT) described herein, e.g., fingolimod or prednisolone. In some embodiments, the composition comprising immunosuppressant is withdrawn from treatment after 1, 2, 3, 4, 5, 5-10, 10-15, 15-20, or more than 20 days of administration. In some embodiments, the mammal is already receiving a course of immunosuppressant at the time of administration of a composition comprising any of the rAAV particles described herein. In some embodiments, the composition comprising any of the the rAAV particles disclosed herein is administered before the immunosuppressant composition. In some embodiments, any of the immunosuppressant compositions disclosed herein is administered before administration of of the rAAV particle composition.

In some embodiments, the therapeutically-effective amount of the rAAV nucleic acid vector in any of the disclosed compositions is an amount of between 10⁶ and 10¹⁴ vector genomes (vgs)/kg of the subject. In some embodiments, the therapeutically-effective amount is greater than 10¹⁴ vector genomes (vgs)/kg subject. In some embodiments, the therapeutically-effective amount is about 10¹¹ vector genomes (vgs)/kg. In some embodiments, the therapeutically-effective amount is 10⁶, 10⁷, 10⁸, 10⁹, 10¹⁰, 10¹¹, 10¹², 10¹³, or 10¹⁴ vgs/kg.

In some embodiments, progression of the autoimmune disease in the mammal is inhibited or reversed for at least 150 days in a subject suffering therefrom that is refractory to other MS therapies, such as one or more standard-of-care MS therapies. In some embodiments, progression of the autoimmune disease in the mammal is inhibited or reversed for at least 150 days, in a subject that is refractory to a small-molecule MS therapy, such as a BTK inhibitor or a pyrimidine synthesis inhibitor. In some embodiments, the subject is human. In some embodiments, the composition or particle comprising the rAAV nucleic acid vector is administered to the subject in a single injection.

In some embodiments, progression of relapse-remitting forms of MS in the mammal is inhibited or reversed for at least 50 days, at least 100 days, at least 150 days, at least 175 days, at least 200 days, or more than 200 days after administration of any of the disclosed rAAV particles or compositions comprising any of the disclosed rAAV nucleic acid vectors to the subject suffering therefrom.

### Example 4 - Further Data From EAE Mouse Model and Assessment of Other Proteins

Animals were injected intravenously via tail vein with 10¹¹ vector particles of AAV8-apoE/hAAT-MOG. The MOG sequence used was murine MOG. It was shown that MOG transgene was expressed in the liver as evidenced by increased amounts of MOG protein (FIG. 11) in samples from the liver.

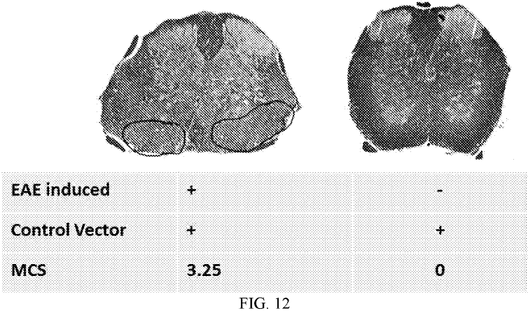

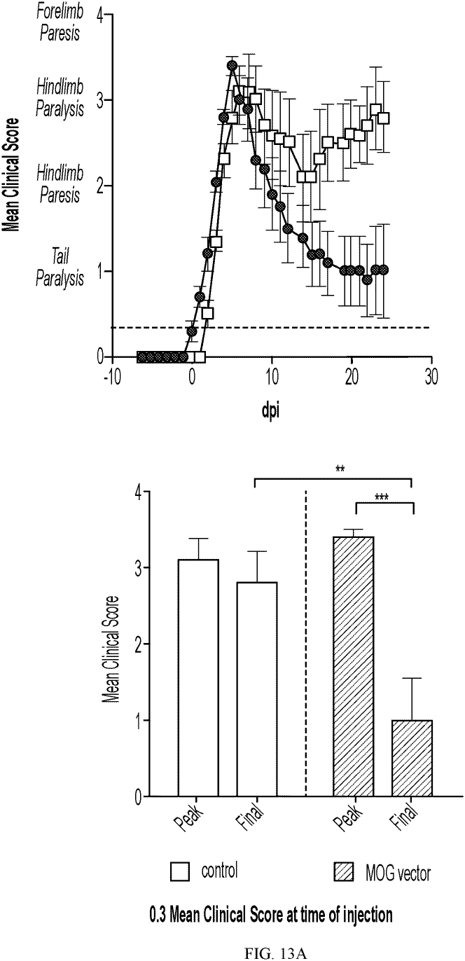

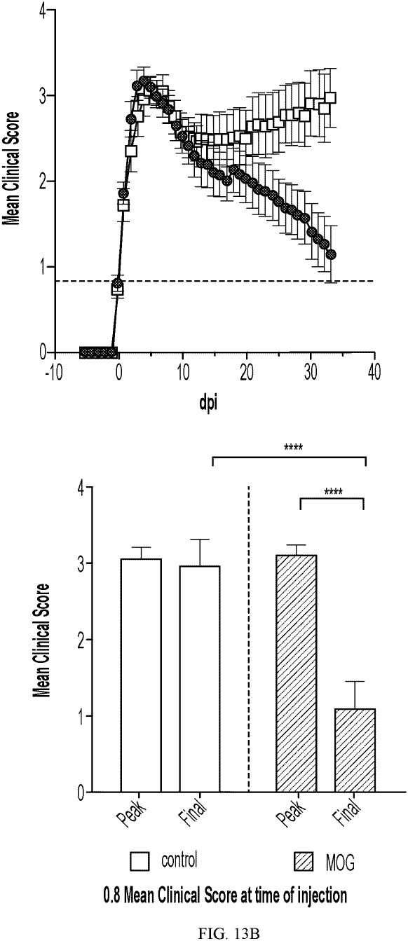

To ensure that AAV did not interfere with the development or progression of EAE in the mouse model described in the other Examples, control AAV8-GFP was injected intravenously into mice. Two weeks later, EAE was induced or not induced. The AAV control vector did not appear to interfere with development or progression of EAE (FIG. 12).

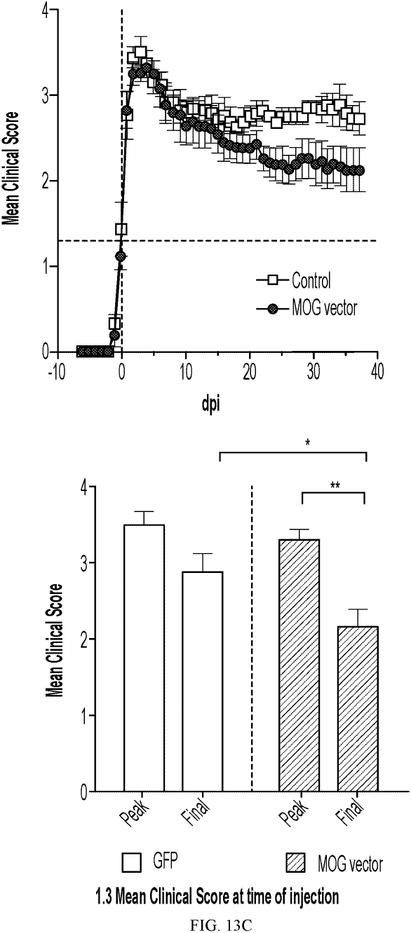

In another study, EAE was induced in C57BL/6 mice. At various times of neurological deficit of mean clinical score (MCS) ~0.3, -0.8, or -1.3, mice received AAV8-MOG or control vector. Mean clinical score was recorded. Even at increasing disease pathology, AAV-MOG vector had significantly reduced neurological deficit compared to control vector treated mice (FIGS. 13A-13C). Bar graphs show statistical significance between final scores and peak-to-final scores.

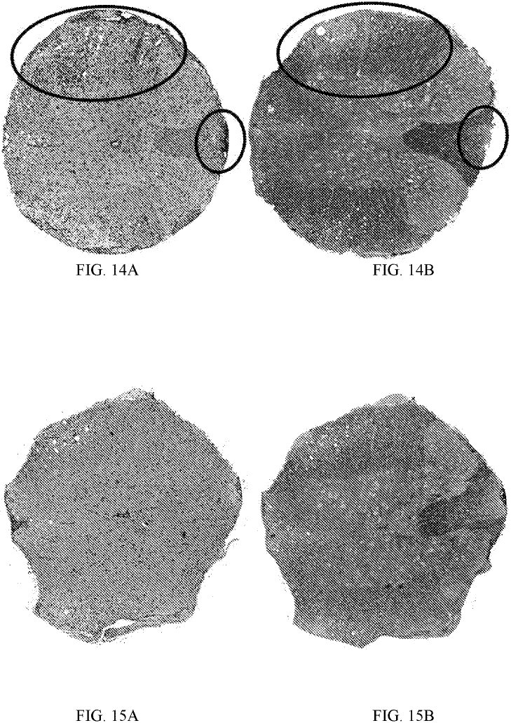

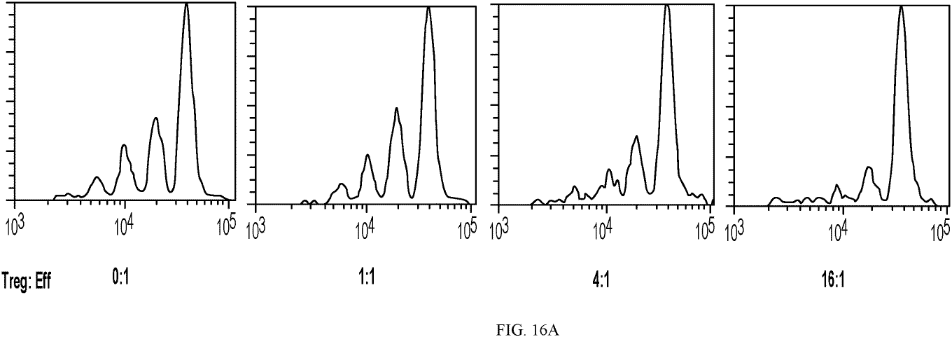

In a further study, serial sections of spinal cord were taken from an EAE-induced female mouse ~35 days after receiving control vector (MCS=4.0). Hematoxylin and eosin stain showed areas of high inflammatory infiltration (FIG. 14A). Luxol fast blue stain showed areas of demyelination (FIG. 14B). In contrast, serial sections of spinal cord from an EAE-induced female mouse ~35 days after receiving AAV-MOG vector (MCS =1.25) showed suppression of inflammation. Hematoxylin and eosin stain showed diminished infiltration (FIG. 15A). Luxol fast blue stain appeared to have less areas of demyelination as a result of the suppression of the inflammation (FIG. 15B).

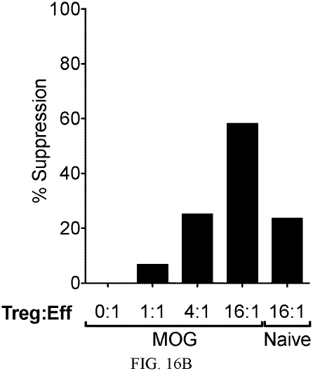

In another study, regulatory T cell (Treg)-mediated suppression was measured by CFDA-SE Cell Tracer. Effector T cells (Teff) were isolated from C57BL/6 mice and labeled with CFDA. Cells were cultured either alone or in the presence of Tregs at a various Treg:Teff ratios. After 72 hours, proliferation was determined by CFDA dilution and flow cytometric analysis. Tregs isolated from spleens of AAV-MOG treated mice were found to be functionally suppressive (FIGS. 16A and 16B).

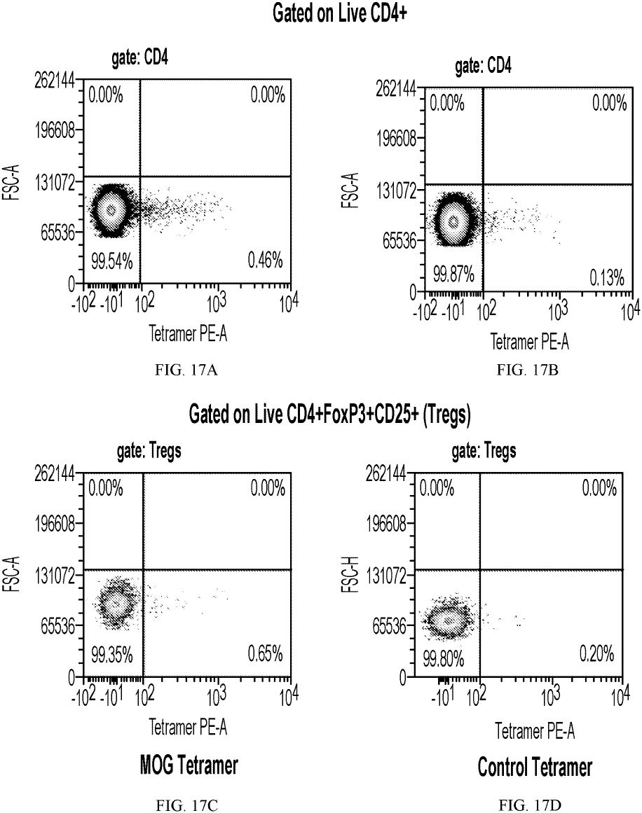

In a further study, the ability of AAV-MOG to induce antigen specific Tregs was assessed. Splenocytes from mice injected with AAV-MOG vector 8 weeks prior showed an increase in frequencies of I-Ab MOG₃₅₋₅₅ Tetramer positive CD4⁺ and Treg+ compared to control tetramer (FIGS. 17A-17D), indicating that AAV-MOG vector induced antigen-specific Tregs.

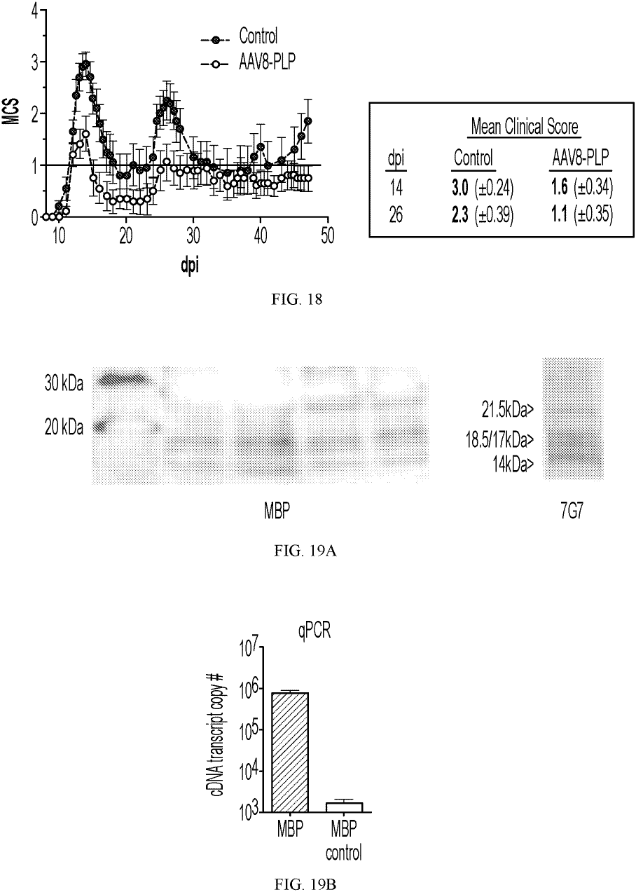

Next, a PLP vector was tested. AAV8-PLP was used for this part of the study. The PLP used was murine PLP. This initial proof-of-concept experiment demonstrated the timeline and clinical scoring for successful induction of PLP/EAE and the potential therapeutic benefit of liver gene transfer. Female SJL mice were injected with AAV8-PLP or control 2 weeks before immunization with 200 µg PLP emulsified in CFA containing 4 mg/ml Mycobacterium tuberculosis. Clinical signs of EAE began ~10 days later, at which time mice were evaluated twice daily. Mice were scored according to the severity of the clinical signs (FIGS. 2). Clearly, mice receiving AAV8-PLP vector had a significant reduction in disease at the peak of onset (FIG. 18). There was also a significant decrease in MCS during relapse (day 26) with fewer relapses overall (FIG. 18). These results show that AAV8-PLP reduced clinical severity in mice with PLP-induced relapsing-remitting EAE. Increased reduction or complete prevention is anticipated with optimization of vector dose and timing.

Lastly, a MBP vector was tested. AAV8-MBP was used for this part of the study. The MBP used was murine MBP. Western blot analysis from protein extracted from liver of mice injected with AAV8-MBP showed an increase in MBP expression (FIG. 19A), which was consistent with an increase in mRNA levels (FIG. 19B).

### Example 5 - Gene Therapy-Induced Antigen-Specific Tregs Inhibit NeuroInflammation and Reverse Disease in a Mouse Model of Multiple Sclerosis

The devastating neurodegenerative disease multiple sclerosis (MS) could substantially benefit from an adeno-associated virus (AAV) immunotherapy designed to restore a robust and durable antigen-specific tolerance. However, developing a sufficiently potent and lasting immune-regulatory therapy that can intervene in ongoing disease is a major challenge and has thus been elusive. This problem was addressed herein by developing a highly effective and robust tolerance-inducing in vivo gene therapy. Using a pre-clinical animal model, a liver-targeting gene transfer vector that expresses full-length myelin oligodendrocyte glycoprotein (MOG) in hepatocytes was designed. It is shown here that by harnessing the tolerogenic nature of the liver, this powerful gene immunotherapy restores immune tolerance by inducing functional MOG-specific regulatory T cells (Tregs) in vivo, independent of major histocompatibility complex (MHC) restrictions. It is demonstrated herein that mice treated prophylactically are protected from developing disease and neurological deficits. More importantly, it is also demonstrated herein that when given to mice with preexisting disease, ranging from mild neurological deficits to severe paralysis, the gene immunotherapy abrogated CNS inflammation and significantly reversed clinical symptoms of disease. This specialized approach for inducing antigen-specific immune tolerance has significant therapeutic potential for treating MS and other autoimmune disorders.

### INTRODUCTION

Multiple sclerosis (MS) is a complex T cell-driven autoimmune disease of the CNS for which there is no known cure. Although the exact etiology is unknown, the disease is thought to result from peripheral activation of myelin-reactive CD4⁺ effector T cells that have escaped immune-regulatory mechanisms.^{39-41,4,74}

Active suppression by regulatory T cells (Tregs) plays a key role in the control of self-antigen-reactive T cells and the induction of peripheral tolerance in vivo.⁴⁰ Unfortunately, abnormalities in the frequency or suppressive function of peripheral CD4⁺CD25⁺FOXP3⁺ Tregs have been observed in various autoimmune diseases, including MS.^{75,76}

An attractive therapeutic strategy for restoring self-tolerance and controlling disease is to selectively induce autoantigen-specific CD4+CD25+FOXP3+ Tregs. Numerous studies have demonstrated the power of Treg-based immunotherapies.^{75,77,78} For example, it has been shown that adoptive transfer of polyclonal CD4+CD25+ Tregs can temporarily prevent or reduce the neurological symptoms of experimental autoimmune encephalomyelitis (EAE), the murine model of MS.⁷⁹ Recent clinical studies have reported that injection of CD4+CD25+ Tregs appears to be a safe and effective cellular treatment in patients with type 1 diabetes and graft-versus-host disease.^{80,81} In an attempt to generate sufficient cells, several ex vivo approaches for expanding CD4+CD25+ Tregs or in vitro induction of Tregs have been explored.⁷⁸ Polyclonal and in vitro antigen-specific Treg expansion are two well-known methods that have been used to generate an adequate amount of CD4+CD25+ Tregs. Unfortunately, there are several obstacles blocking the development of large-scale ex vivo or in vitro antigen-specific Treg expansion techniques.⁸²

An alternative and efficient in vivo approach for inducing Ag-specific tolerance is through ectopic expression of an antigen in the liver.^{46,83} Leveraging the tolerogenic nature of the liver, hepatic gene transfer has successfully been used to induce robust transgene tolerance in large- and small-animal disease models.^{6,84} Viral vectors such as adeno-associated virus (AAV) have emerged as an effective vehicle for in vivo delivery of therapeutic genes to various tissues and are currently being used in multiple phase I/II clinical trials (see ClinicalTrials.gov).

In this disclosure, it was demonstrated that hepatic gene therapy with an AAV vector containing the full DNA coding sequence for the neuroprotein, myelin oligodendrocyte glycoprotein (MOG), can prevent development of and reverse preexisting EAE. The vector therapy resulted in the induction/expansion of antigen-specific FOXP3+ Tregs. When vector is administered prophylactically, mice were protected from developing EAE disease. When administered to mice exhibiting mild-to-moderate neurological deficits, vector alone was effective at reversing both clinical and pathological signs of disease. When combined with a short course of immune suppression, the AAV immunotherapy can rescue mice from fatal end-stage EAE disease and restore mobility after exhibiting severe paralysis.

### Experimental Methods

Animal Strains. Female (9- to 12-week-old) inbred C57BL/6 and C57BL/6-Tg (Tcra2D2,Tcrb2D2), 1Kuch/J (MOGTCR 2D2), and B6.129(Cg)-Foxp3tm3(DTR/GFP)Ayr/J (FOXP3gfp+) mice were purchased from Jackson Laboratories (Bar Harbor, ME, USA). All procedures involving animals were carried out in accordance with the guidelines of the University of Florida Institutional Animal Care and Use Committee (IACUC).

Vector Production. A recombinant AAV8 vector expressing full-length MOG under a hepatocyte-specific promoter was produced by the method of transfection using anionic liposomes as a transfection reagent into human embryonic kidney (HEK293) cells, below passage 50. Two plasmid DNAs—recombinant construct flanked by the AAV inverted terminal repeats (iTRs), pAAV-Apolipoprotein E (ApoE)/hAAT-MOG, and a helper plasmid for AAV8 serotype (pDG8) mixed in equimolar amount—totaling 90 µg per 15 cm plate were added to each plate containing ~1 × 107 cells. Virus was recovered from both cells and medium. Medium was collected on days 2 and 4 post-transfection, with consequent virus precipitation with 40% polyethylene glycol (PEG)8000/2.5 M NaCl solution. Cells were resuspended in 20 mM Tris/HCl (pH 8.5)/15 mM NaCl lysis buffer, 10 mL per 1-2 × 108 cells. Cells were lysed by one-time freeze/thaw cycle and three rounds, 1 min each, of sonication on ice. Virus pelleted by PEG/NaCl was processed similarly to the virus recovered from the cells and combined. Clarified lysates ran on a step iodixanol density gradient⁸⁵ and dialyzed/concentrated on Apollo 20 spinning devices. The titer of each preparation was estimated using a dot-blot assay.

Induction of EAE. Mice were immunized by subcutaneous injection of MOG₃₅₋₅₅ in CFA (Hooke Labs, Lawrence, MA, USA). Pertussis toxin (PT) 200 ng (Hooke Labs, Lawrence, MA, USA) was injected intraperitoneally (i.p.) 6 and 24 hr later. The clinical symptoms of EAE were checked daily and graded on a clinical score of 0-5: 0, no clinical signs; 0.5, partially limp tail; 1.0, paralyzed tail; 2.0, loss of coordinated movement and hind-limb paresis; 2.5, one hind limb paralyzed; 3.0, both hind limbs paralyzed; 3.5, hind limbs paralyzed and weakness in forelimbs; 4.0, forelimbs paralyzed (quadriplegia); and 5.0, moribund. Mice had to reach inclusion criteria of an MCS ≥2.0 to be included in the study group. Mice would be euthanized if an MCS ≥4.0 was maintained for 48 hr, as per IACUC policy.

Vector Administration. To examine the prophylactic effect of vector administration, 7- to 9-week-old female C57BL/6 mice were injected with 1011 vector genomes (vg) of AAV8.MOG or control vector. Two weeks later, EAE was induced. To evaluate the therapeutic effect of vector administration, EAE was induced first in 9- to 11-week-old mice before the administration of vectors. As mice reached the targeted/indicated severity of disease they were injected with 1011 vg of AAV8.MOG or control vector (scAAV.GFP or sham/PBS) via the tail vein. Rapamycin (LC Laboratories, Woburn, MA, USA) was dissolved in a vehicle solution containing (0.2% w/v) carboxymethyl-cellulose sodium salt (C-5013) and (0.25% v/v) polysorbate-80 (P-8074) (Sigma, St. Louis, MO, USA) in distilled water and stored at 4° C. protected from light according to the manufacturer’s instructions. Rapamycin (5 mg/kg) was given i.p. as indicated for a total of three and five doses beginning on the day of vector administration.

Gene Expression. Messenger RNA was isolated from 30 mg of liver samples harvested from mice that had received vector 2 weeks earlier using the RNeasy kit (QIAGEN, Valencia, CA, USA). Real-time qPCR was performed in duplicate using RT2 qPCR Primer Assay for Mouse MOG (QIAGEN) according to the manufacturer’s protocols. A MyIQ iCycler fluorescent detection system with iQ5 operating software Version 2.0 (Bio-Rad Laboratories, Hercules, CA, USA) was used to generate and analyze data. All gene expression was compared with that of glyceraldehyde-3-phosphate dehydrogenase.⁸⁶

Flow Cytometry. Peripheral blood cells or splenocytes harvested from mice and processed to produce single-cell suspensions were stained with antibodies to CD3 (145-2C11), CD4 (RM4-5), CD25 (PC61), CD8 (53-6.7), B220 (RA3-6B2), CD44 (IM7), and CD62L (MEL14) (BD Biosciences, San Jose, CA, USA). Class II MHC tetramers included MOG38-49/I-Ab class II MHC (GWYRSPFSRVVH) and h.CLIP87-101 (PVSKMRMATPLLMQA), and were provided by the NIH Tetramer Core (Emory University, Atlanta, GA, USA). Red blood cell lysis was performed with VersaLyse (Beckman Coulter, Brea, CA, USA). Intracellular staining for FOXP3 was performed using the FOXP3 staining kit (eBioscience, San Diego, CA, USA). Samples were analyzed on an LSR-II flow cytometer (BD Biosciences) and post-analyzed using FCS Express 4 (Denovo Software, Los Angeles, CA, USA).

In Vitro Suppression Assay. Spleens from FOXP3gfp+ mice that received vector no less than 2 weeks earlier were homogenized and enriched for CD4+ T cells by magnetic depletion of non-target cells over an LS column (Miltenyi Biotec, San Diego, CA, USA). GFP+ cells, representing the Treg population of CD4+ T cells (~10%), were further isolated using the FACSAria II cell sorter (BD Biosciences). Splenocytes isolated from 2D2-MOGTCR mice were labeled with CellTrace Violet (Invitrogen, Carlsbad, CA, USA) according to the manufacturer’s protocol. CD4+FOXP3GFP+ Tregs and CellTrace Violetlabeled responder splenocytes were seeded at the indicated effector/responder ratios in complete 5% RPMI media containing 1 µg/mL MOG35-55 peptide for 72 hr at 37° C. Cells were resuspended and stained with anti-CD4 antibody to assess proliferation of responder CD4+ T cells. GFP was used to discriminate between responder cells and Tregs. Proliferation was determined by quantitating CellTrace Violet fluorescence intensity relative to the parent population of unstimulated responder cells (0% proliferation) and stimulated cells incubated without Tregs (100% proliferation). Percentage of CD4+ responder T cell proliferation was determined using FCS Express 4.

Analysis of Plasma Samples. Plasma was analyzed for anti-MOG IgG1 and IgG2c by ELISA as previously described.¹²

Western Blot. Protein was extracted from liver tissue using T-PER Tissue Extraction Reagent (Thermo) in the presence of Halt Protease Inhibitor (Thermo). Total protein concentration was measured using the bicinchoninic acid protein assay (Pierce). Samples were separated on 4%-20% Mini-PROTEAN TGX gels (Bio-Rad) and transferred to polyvinylidene fluoride (PVDF) membrane following standard protocols. After blocking, the membrane was incubated for 1 hr at room temperature with antibody against MOG or β-actin in 1% fat-free dry milk in 1× tris-buffered saline with tween (TBST). HRP-conjugated secondary antibody was used for signal detection with the ECL 2 Western Blotting Substrate (Pierce).

Histopathology. For histopathological analysis of the spinal cord, formalin-fixed, paraffin-embedded, 5 µm sections were stained with Luxol Fast Blue or H&E by standard procedures.

Statistical Analysis. Results are reported in figure legends as mean ± SEM, unless otherwise stated. Statistical significance was determined using GraphPad Prism software (La Jolla, CA, USA). The p values are reported as indicated.

Study Approval. All studies were in accordance with protocols approved by IACUC at the University of Florida, Gainesville.

### RESULTS

### Hepatic Gene Transfer With AAV8.MOG Induces Immunosuppressive MOG-Specific Tregs

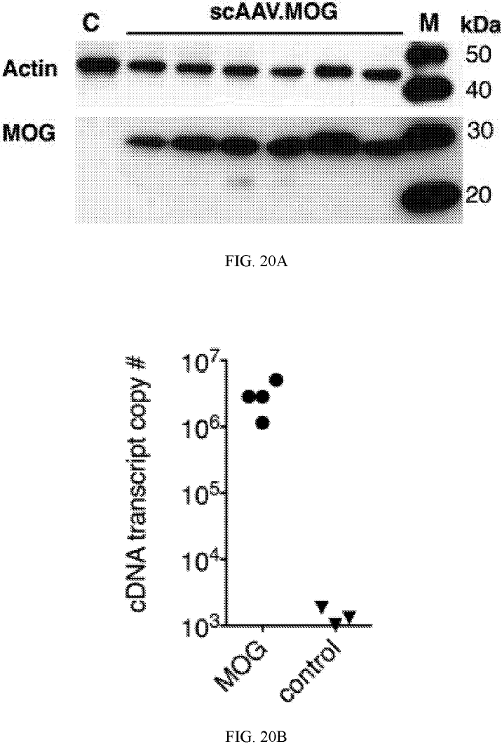

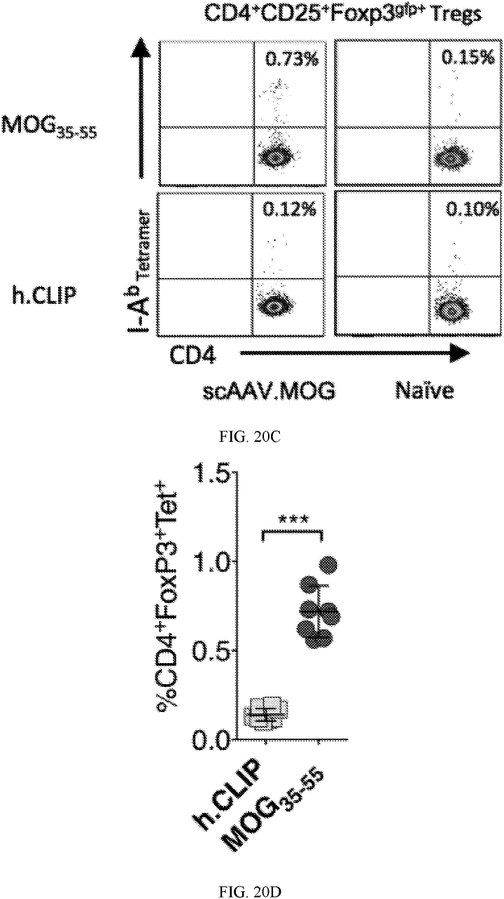

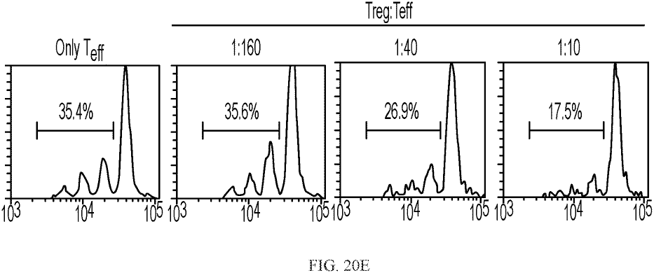

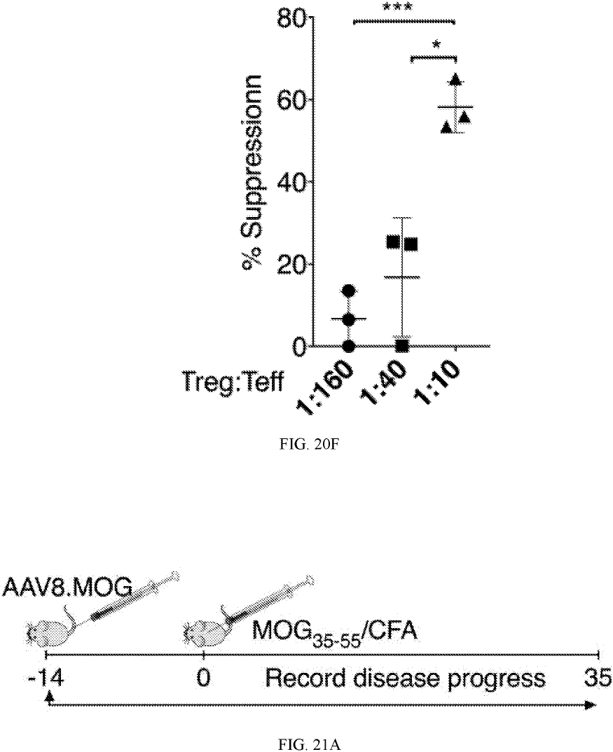

To study the ability of the liver to induce immune tolerance to a transgene protein, an AAV8 vector was engineered to contain the full coding sequence (CDS) of the neuroprotein MOG, which was placed under control of a liver-specific promoter. To establish that AAV8.MOG can transduce hepatocytes and will stably express the non-secreted neuroprotein, C57BL/6 mice were systemically injected with a single dose of the vector (10¹¹ vector genomes). Western blot and real-time qPCR analysis of liver lysates from tissue harvested 2 weeks later confirmed vector transduction and hepatocyte expression of MOG (FIGS. 20A and 20B).

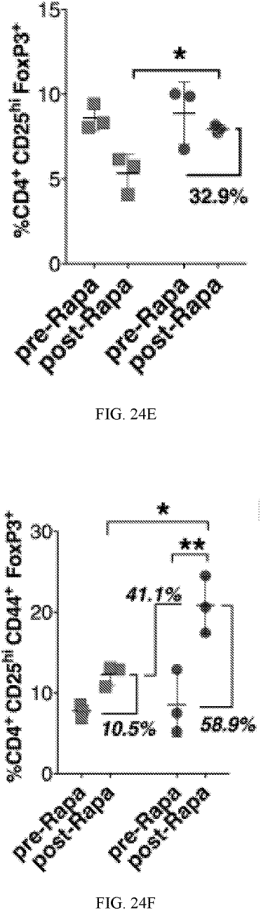

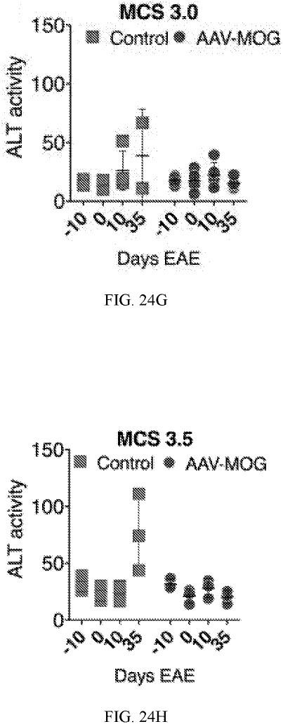

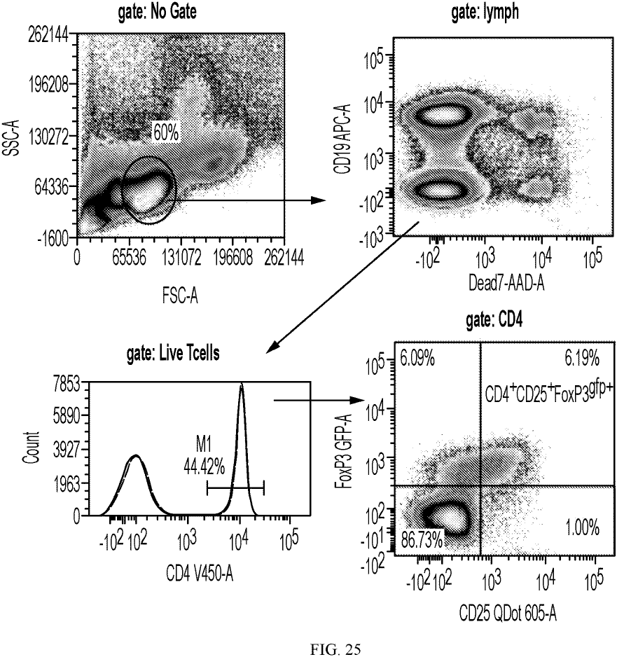

Although MOG protein accounts for only 0.05%-0.1% of total myelin proteins, it is reported to induce a more potent T cell response than other myelin antigens in patients with MS.^{87,88} To complicate matters, a loss of immune tolerance because of deficits in either Treg numbers or their function has been observed in autoimmune and inflammatory diseases, including MS.⁷⁴ Previously, in a model used for protein replacement therapy, the notion that hepatocyte expression induces transgene (Tg)-specific Tregs could only be indirectly established.⁷ Here, an experimental system was developed that allowed for direct determination of the frequency of MOG-specific FOXP3+ Tregs. Using a transgenic C57BL/6 Foxp3-EGFP reporter mouse that expresses EGFP under the control of the mouse Foxp3 promoter (FOXP3-gfp+), in combination with a MOG₃₈₋₄₉/I-Ab major histocompatibility complex (MHC) tetramer, allowed for the direct identification of AAV8.MOG-induced Tg-specific CD4+ Tregs (FIGS. 20C and 20D). To do so, freshly isolated splenocytes from mice that had previously received AAV8.MOG vector were chosen for analysis. This allowed for the determination of the real-time frequency of tetramer-specific cells as opposed to values amplified by ex vivo restimulation.⁷² Multi-parametric flow cytometry revealed a significantly higher I-Ab MOG35-55 tetramer+ frequency from CD4+CD25+FOXP3-gfp+ gated cells compared with the control (h.CLIP/I-Ab) tetramer (p < 0.0001) (FIGS. 20C and 20D; FIG. 25). Similarly, age-matched naive reporter mice failed to bind either tetramer at levels above that of control mice, ruling out potential non-specific binding (FIG. 20C). These findings provide direct and unambiguous evidence that liver-directed AAV induces transgene-specific Tregs in mice, further confirming that hepatic expression of a full-length transmembrane neuroprotein can indeed drive in vivo induction of antigen-specific Tregs.

Next, to test whether the vector-induced MOG-specific Tregs were functional, the capacity of FAC-sorted CD4+FOXP3gfp+ Tregs harvested from the spleens of AAV8.MOG tolerized mice, or age-matched naive mice, to suppress the proliferation of MOG-specific effector T cells was assessed when co-cultured in the presence of the immunodominant MOG₃₅₋₅₅ peptide. Indeed, at a 1:10 Treg/effector T cell (Teff) ratio, the vector-induced Tregs suppressed 58% of the effector cell proliferation. This was nearly three times more effective than naive polyclonal Tregs (FIGS. 20E and 20F). These results demonstrate that hepatocyte expression of a non-secreted transmembrane neuroprotein delivered by an AAV8 vector induces functionally suppressive MOG-specific Tregs in vivo.

### Pre-Treatment With AAV8MOG Vector Prevents EAE Induction

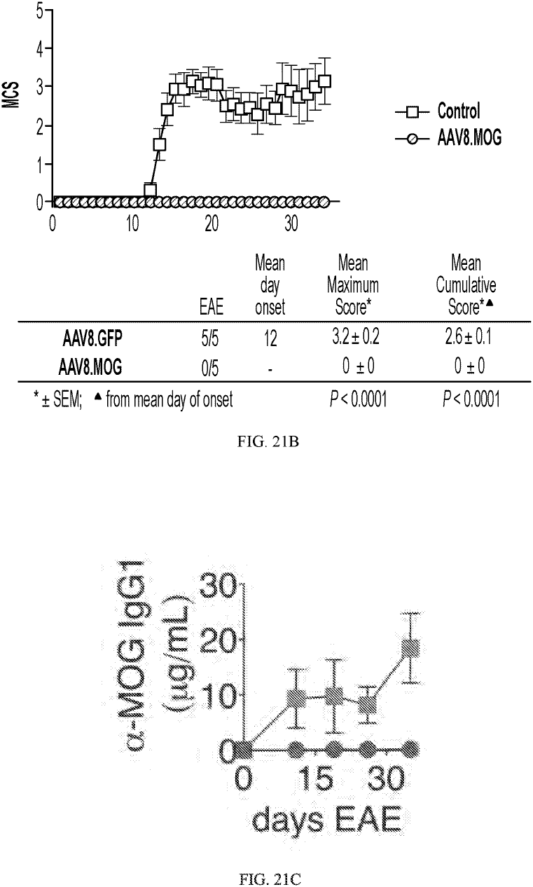

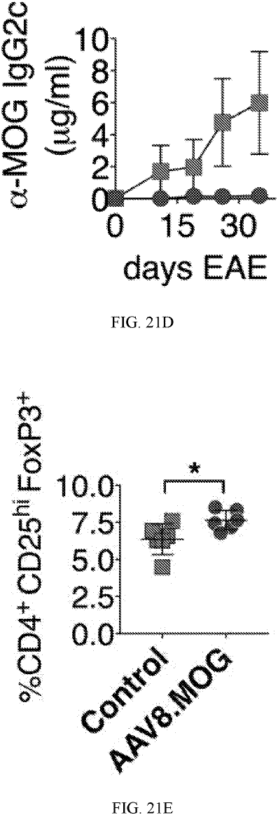

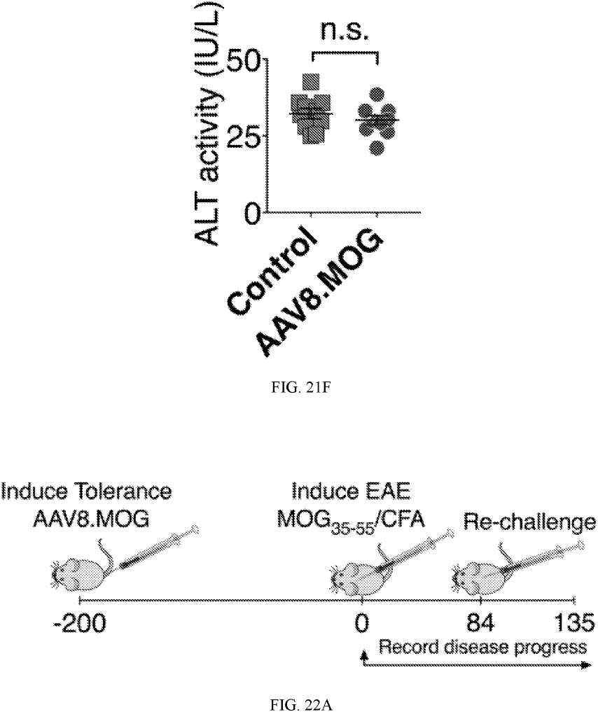

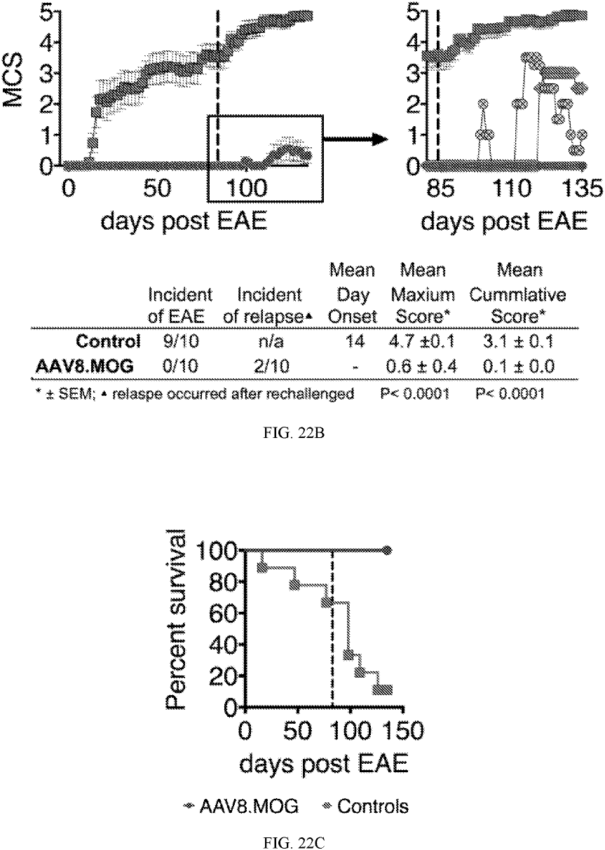

Next, experiments were conducted to examine whether pre-treatment with AAV8.MOG would induce transgene-specific immune tolerance and protect susceptible mice from developing EAE (FIG. 21A). AAV8.MOG or AAV8.GFP (irrelevant transgene control) vector was administered to cohorts of mice. Two weeks later, mice were immunized with MOG35-55 emulsified in complete Freund’s adjuvant (CFA) to induce EAE. Mice were monitored for signs of neurological deficits using a five-point scale as described (Table 1). Beginning 10 days after EAE induction, mice receiving control vector developed severe neurological impairments (maximum mean clinical score [MCS]: 3.15 ± 0.2) (FIG. 21B). Disease progression was also associated with increasing anti-MOG35-55 immunoglobulin (Ig) G1 and IgG2c antibody titers (FIGS. 21C and 21D). In contrast, mice that received AAV8.MOG were protected and failed to develop clinical signs of EAE or produce MOG35-55-specific antibody responses (FIGS. 21B-21D). Notably, neurological deficits in control mice continued to increase in terms of both maximum and cumulative EAE scores until they developed severe paralysis and needed to be humanely euthanized.

Next, the frequency of FOXP3+ Tregs in peripheral blood mononuclear cells was evaluated. Flow cytometry analysis results showed that mice treated with AAV8.MOG had a small but significant increased frequency of CD4+CD25hiFOXP3+ Tregs in peripheral blood mononuclear cells (PBMCs) compared with control mice (FIG. 21E), further supporting that AAV hepatic gene therapy administration selectively expands FOXP3+ Treg populations and induces tolerance to the encoded transgene antigen.⁶

Although various proteins have been safely expressed in the liver following AAV gene transfer,⁸⁹ evaluating the long-term stability of MOG expression in hepatocytes in the context of induced EAE was of interest. Elevations in serum/plasma alanine aminotransferase (ALT) enzyme level are routinely used in the clinic to screen for liver disease and cell-mediated immunity directed against AAV-transduced hepatocytes.^{90,91} Analysis of plasma ALT activity in mice ~4 months after receiving gene transfer revealed no significant difference between AAV8.MOG-treated and age-matched naive C57BL6 mice, indicating that AAV8.MOG did not induce chronic liver disease (FIG. 21F). Additionally, hepatocyte expression of MOG persisted in the mice that received AAV8.MOG until termination of experiment at 200 days after EAE induction (FIG. 20A). Notably, throughout this protracted timeline, mice never developed any observable signs of neurological disability or general distress, suggesting that hepatocyte expression of MOG does not provoke any deleterious immune responses. Collectively, these data demonstrate that prophylactic administration of liver-directed AAV8.MOG produces long-term stable hepatocyte expression of MOG that has an immuno-suppressive effect capable of preventing the development of EAE.

TABLE 1

Description of the Mean Clinical Score (MCS) five-point scale

| Score | Clinical Presentation |
|---|---|
| 0.0 | no clinical signs |
| 0.5 | partial paralysis/limp tail |
| 1.0 | paralyzed tail |
| 1.5 | impaired coordination/balance |
| 2.0 | hind-limb paresis |
| 2.5 | one hind limb paralyzed |
| 3.0 | hind-limb paralysis (paraplegia) |
| 3.5 | hind limbs paralyzed and forelimb paresis |
| 4.0 | hind-limb and forelimb paralysis (quadriplegia) |
| 5.0 | moribund/dead* |

*Mice euthanized or found deceased were recorded as 5 for remainder of time.

### The Immune Tolerance Induced by AAV8MOG is Robust

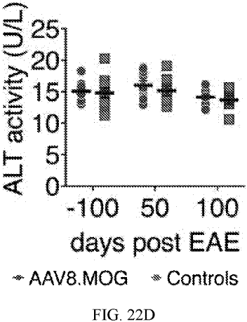

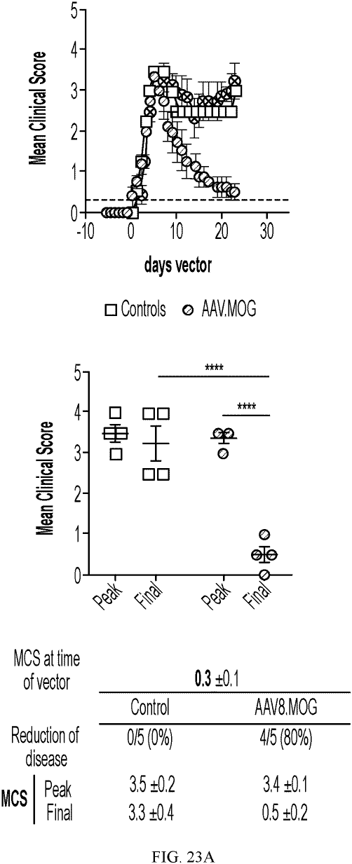

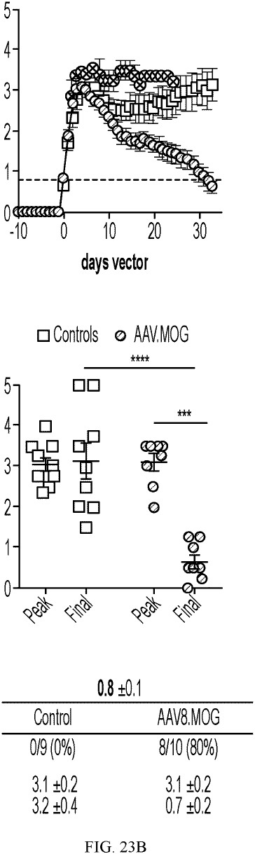

Next, the possibility that a single vector injection could provide long-term hepatic transgene expression and still induce immune tolerance was evaluated. Two cohorts of mice were injected, intravenously, with either AAV8.MOG or PBS/sham (FIG. 22A). EAE was induced in both cohorts of animals ~200 days later with MOG₃₅₋₅₅/CFA. Mice were then monitored daily, and collections of plasma and lymphocytes were obtained every 2 weeks for analysis and Treg staining. Even though vector was given over 7 months earlier, mice that received AAV8.MOG failed to develop any signs of EAE disease, whereas the age-matched control mice began exhibiting neurological deficits at day 14, which rapidly increased in severity (FIG. 22B), and began to succumb to disease as early as 16 days after EAE induction (FIG. 22C). These results demonstrate that vector-induced immune tolerance is stable and can be maintained long term.

In EAE⁹²⁻⁹⁵ and other models of protein replacement gene therapy,^{6,15} long-term induction of tolerance is often confirmed by re-challenging the mice. To further demonstrate the robustness of the immunotherapy described herein, the ability of the AAV8.MOG treatment to maintain tolerance and prevent disease following a second attempt to induce MOG-specific EAE was evaluated. Almost 3 months after the initial EAE induction (9.5 months from AAV8.MOG induction of tolerance), both groups of mice were re-challenged with MOG35-55 and monitored for development of or change in clinical signs. In the control mice, disease escalation occurred rapidly (FIG. 22B). Within 15 days, half of the re-challenged control mice succumbed to disease, whereas 100% of the AAV8.MOG-treated mice survived (FIG. 22C). However, at 16 and 37 days after secondary challenge, two mice developed a slow relapsing-remitting disease (FIG. 22B, right). Nonetheless, 80% of the mice that received AAV8.MOG vector months earlier showed absolutely no signs of EAE or liver disease over the course of the experiment (FIGS. 22B-22D). The disease escalation in the control mice confirmed that vector-treated mice were indeed tolerized and not simply protected via a vaccination mechanism. Thus, these data clearly demonstrated that AAV8.MOG protection is indeed stable and robust.

### AAV8MOG Immunotherapy Reverses Established EAE Disease

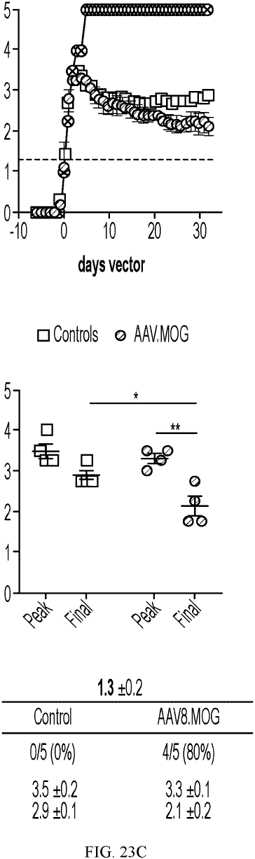

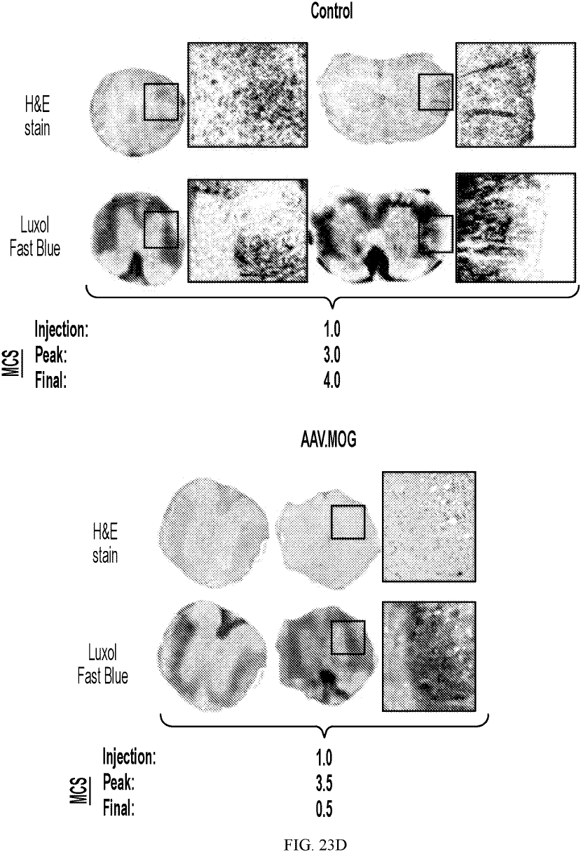

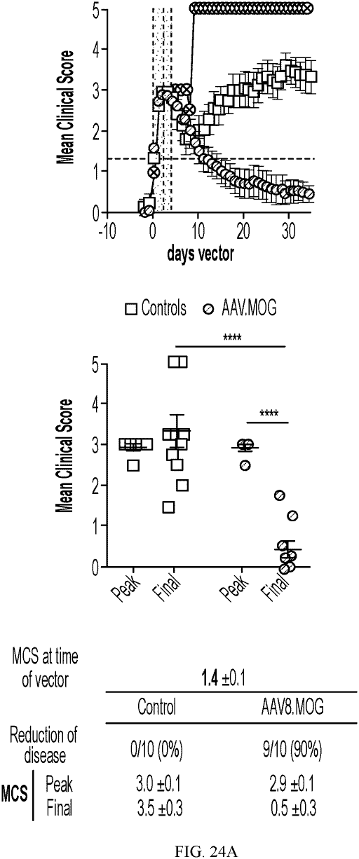

The early symptoms of MS are often minor and overlooked. Diagnosis is usually made after the first clinically isolated syndrome (CIS), which is defined as an episode of neurological deficit that lasts at least 24 hours and is caused by inflammation or demyelination.⁹⁶ In terms of rate and severity of disability, disease progression is highly variable and difficult to predict, which often results in a diagnosis well after disease has been established. Therefore, experiments were conducted to investigate whether induction of Ag-specific tolerance following AVV8.MOG immunotherapy would be effective in diminishing or reversing disease in mice during progressive stages of neurological impairment. In this series of experiments, EAE was induced in age-matched mice before being treated with vector. As the mice developed signs of neurological impairment, they were divided in an alternating fashion into two different groups so that the baseline clinical scores would be comparable between the groups (referred to as rolling enrollment). As mice reached the target MCS, they were injected with either AAV8.MOG or PBS/sham vector (FIGS. 23A-23C). In the first cohort, mice received treatment early in the disease process as they began to lose tail tonality (FIG. 23A). Both groups of mice continued to develop severe paralyzing EAE by day 7 (peak MCS: ~3.5). Strikingly, beginning around day 8, all but one mouse that was treated with a single injection of AAV8.MOG began to exhibit a significant reversal of clinical symptoms (final MCS: 0.5 ± 0.3). In contrast, control mice proceeded to develop severe neurological disabilities (final MCS: 3.3 ± 0.4). In the next iteration, the ability of AAV8.MOG immunotherapy to reverse moderate disease was evaluated by withholding treatment until mice exhibited complete tail paralysis (MCS: ~1). Like before, both groups of mice rapidly developed severe EAE with hind-leg paralysis (FIG. 23B). After a brief remission, control mice relapsed and developed severe ascending paralysis (final MCS: 3.2 ± 0.4). In contrast, AAV8.MOG-treated mice went into a nearly complete remission and regained use of their hind legs (final MCS: 0.7 ± 0.2) (FIG. 23B).

Lastly, the ability of AAV8.MOG to induce tolerance and abrogate disease in mice with even more advanced preexisting disease was further probed. Following induction of EAE, AAV8.MOG immunotherapy was withheld until disease advanced and mice presented with complete tail paralysis with hind-leg inhibition and loss of fine motor coordination that affected their gait and balance (combined MCS: 1.3 ± 0.2) (FIG. 23C). Mice continued to develop severe EAE with hind-leg paralysis, which critically impeded their ability to freely move around the cage and obtain food (peak MCS: ≥3.3). By day 30, mice that received AAV8.MOG immunotherapy had a significantly greater reduction in clinical scores compared to control mice (p = 0.0412). Although not as robust as previously seen, these results have substantial clinical relevance. Notably, all the mice that responded to the gene immunotherapy regained the ability to freely ambulate, whereas control mice continued to have hind-leg paralysis.

### Inflammation in the Spinal Cord of Treated Mice

In EAE, the spinal cord is the primary site of encephalitogenic effector cells and demyelination, and the degree of neurological impairment is related to the magnitude of inflammation during the early stages of the disease.¹ To determine whether the amelioration of neurological deficits was associated with a reduction in encephalitogenic inflammation and/or demyelination, serial sections from multiple regions of spinal cords from mice that received AAV8.MOG were compared to control mice for pathological differences 35 days after receiving vector. Histological examination showed that non-tolerized control mice had numerous foci of cellular infiltrates that were co-localized to areas of demyelination within the white matter (FIG. 23D). In contrast, there was an absence of inflammatory lesions within the spinal cord of the mice treated with AAV8.MOG. These findings were consistent across the other treatment groups, as well as with prior literature.^{46,97} These results suggest that AAV gene immunotherapy reverses the clinical symptoms associated with EAE disease through a mechanism that suppresses tissue-specific inflammation.

### Transient Immune Suppression Enhances AAV8MOG Immunotherapy in EAE

It has been shown in an EAE model that Tregs may accumulate in the target tissue but are non-suppressive.⁷² The failure to suppress the effector response is believed to be associated with the localized inflammation causing a Th1/Th17 microenvironment within the CNS. As suggested by the diminished impact of AAV8.MOG treatment in mice with delayed treatment seen in FIG. 23C, this pro-inflammatory microenvironment may limit the effectiveness of the induced Tregs, especially at the height of inflammation. To overcome this limitation, it was hypothesized that successful treatment may require adjunct immune suppression to modulate the pro-inflammatory environment within the CNS.^{98,99} To address this, the immunosuppressive drug rapamycin was investigated. Rapamycin has been used to suppress graft refection in organ transplantation, and its safety and efficacy have been evaluated for use in humans with MS.⁶⁹ In general terms, rapamycin has a potent anti-proliferative effect on antigen-stimulated effector T cells, while simultaneously allowing expansion of CD4+CD25+FOXP3+ Tregs, making it an ideal choice.^{100,101}

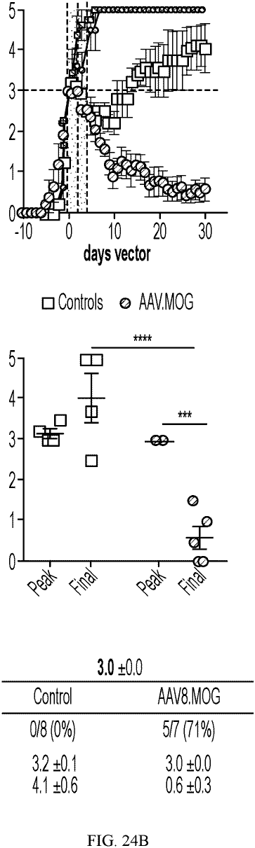

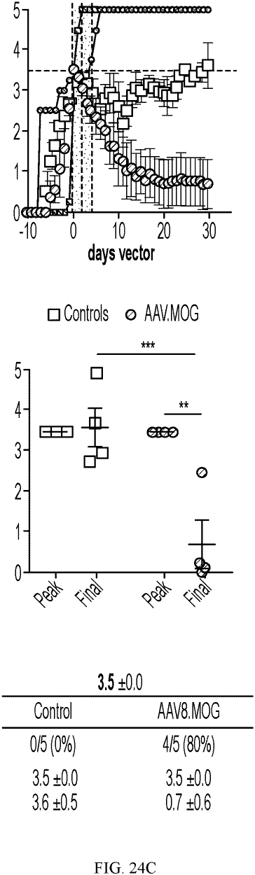

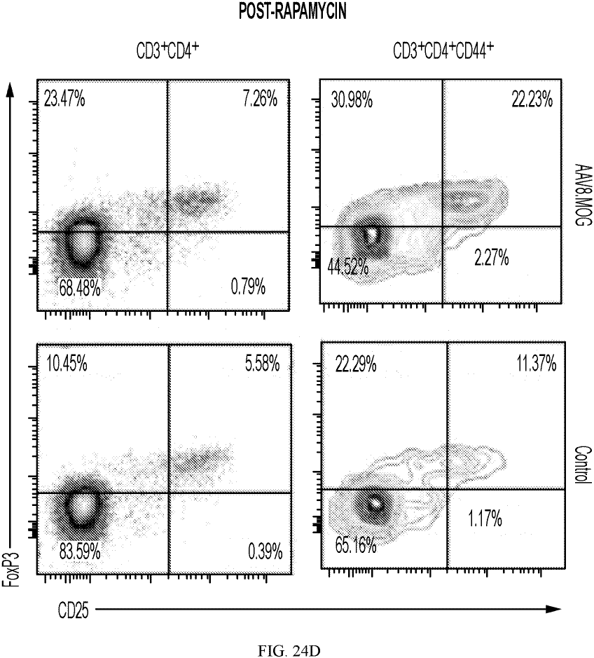

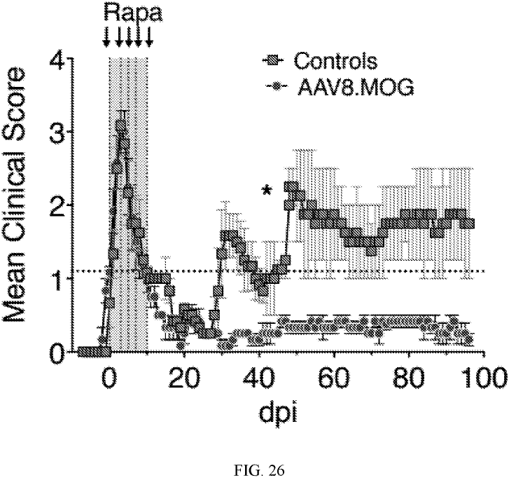

To test the hypothesis, the experimental parameters that previously produced the smallest degree of disease reversal were reestablished (FIG. 23C). EAE was induced and AAV8.MOG treatment was withheld until mice developed complete tail paralysis with hind-leg paresis (MCS: 1.4 ± 0.1, combined). Immediately after being treated with either AAV8.MOG or PBS/control, all mice received an intraperitoneal injection of rapamycin (5 mg/kg). Subsequently, mice received two additional doses of rapamycin (5 mg/kg) 48 hr apart (FIGS. 24). As expected, EAE disease progressed quickly and both groups of mice developed severe neurological deficits and paralysis (peak MCS: 2.9-3.0) (FIG. 24A). Within 72 hr of receiving the rapamycin, both AAV8.MOG-treated and control mice responded to the immunosuppression and displayed signs of remission (a sustained reduction in MCS ≥1).⁶⁹ However, by day 10, 100% of the control mice had relapsed and rapidly developed end-stage EAE disease (final MCS: 3.5 ± 0.3). In contrast, neurological deficits in AAV8.MOG-treated mice continued to decrease, and all but one animal (90%) achieved complete remission (final MCS: 0.5 ± 0.3) (FIG. 24A). Additionally, in a separate experiment, mice that received the AAV8.MOG vector/rapamycin combination remained symptom free (final MCS: 0.2 ± 0.1) until termination of the experiment at ~100 days after EAE (FIG. 26).

Next, the effectiveness of the combined immunotherapy in late and end-stage EAE disease was tested. In these cohorts, EAE disease was induced as before and allowed to develop until the mice began exhibiting complete tail and hind-limb paralysis (MCS: 3.0 ± 0.0) (FIG. 24B) or borderline quadriplegia (hind-limb paralysis with forearm paresis that prevents the mouse from righting itself when placed on its back) (MCS: 3.5 ± 0.0) (FIG. 24C) before AAV8.MOG/rapamycin treatment was administered. Remarkably, mice that received the AAV8.MOG/rapamycin immunotherapy, 71% (FIG. 24B) and 80% (FIG. 24C), respectively, responded to the treatment and went into near-complete remission (MCS: >1) by day ~30. In contrast, after transiently responding to the rapamycin, the control mice relapsed into severe paralyzing or fatal EAE disease (FIGS. 24B and 24C). Notably, in both groups, a limited number of the animals failed to respond to rapamycin immunosuppression, suggesting the disease process was beyond the point of rescue.

### Hepatocyte Expression of MOG in Combination With Rapamycin Promotes Treg Expansion of Peripheral Tregs and Reverses EAE

Rapamycin blocks the activation of a serine/threonine protein kinase called mammalian target of rapamycin (mTOR), which has a potent anti-proliferative effect on antigen-stimulated effector T and B cells. This results in selective reduction of T helper (Th) 1, Th2, and Th17 cells while simultaneously allowing the expansion of Ag-specific Tregs.¹⁰⁰ To determine whether rapamycin treatment enhanced the induction of tolerance and cellular responses during AAV8.MOG immunotherapy, the frequency of Tregs from AAV8.MOG/rapamycin-treated mice was compared with rapamycin-only control mice (FIGS. 24D-24F). Phenotypic analysis revealed no significant difference in the percentage of total CD4+CD25hiFOXP3+ Tregs obtained from peripheral blood of AAV8.MOG tolerized mice, compared with that of control mice before rapamycin treatment. In contrast, when analyzed after the final rapamycin dose on day 10, there was an ~33% difference in total Tregs between control mice receiving rapamycin alone and AAV8.MOG-treated animals (FIGS. 24D and 24E).

CD44 is a cell-surface glycoprotein involved in cell-to-cell interactions that are important in activation, migration, and apoptosis. Its relative expression has been associated with FOXP3 expression and Treg function, and can be used to identify activated Tregs.^{102,103} Similar to activated effector or memory CD4+ T cells, activated Tregs also express high levels CD44.¹⁰³ Restricting the analysis to activated Tregs (CD4+CD44+ CD25hiFOXP3+) revealed a 58.9% increase in Tregs in mice that received rapamycin and AAV8.MOG immunotherapy (FIGS. 24D and 24F). In contrast, only a 10.5% increase was seen in rapamycin-only-treated mice.

Plasma ALT levels were also monitored as an indicator of liver damage and failure of therapy. As reported above, the level of ALT activity detected in AAV8.MOG-treated mice and control mice was unremarkable throughout the rapamycin treatment window (FIGS. 24G and 24H). However, at 35 days post-treatment the control mice had a significant increase in plasma ALT levels that corresponded with an increase in clinical score (MCS: 3.6 ± 0.5, final). Based on the profound level of neurological impairment the control mice were experiencing, the significant rise in ALT is indicative of liver toxicity associated with end-stage organ failure (FIGS. 24B and 24C).

Collectively, these findings demonstrate that transient immunosuppression with rapamycin has a synergistic effect on AAV8.MOG immunotherapy that selectively induces in vivo expansion of Tregs and restores tolerance in an antigen-dependent manner.¹⁰⁰

MS is a complex autoimmune disease that has no cure. Early diagnosis and aggressive treatment with immunomodulating agents can lower the relapse rate and slow progression. However, these treatments are generally non-specific and risk significant side effects with long-term use.¹⁰⁴ Newer disease-modifying therapies that target specific immune responses or target specific CNS antigens have shown potential, but various experimental limitations have prevented clinical translation.^{77,105,106}

Tregs are an essential component in preventing autoimmunity and controlling responses to alloantigens. A disruption in the homeostasis of tolerance in a variety of autoimmune diseases, including MS, may result from a substantial decrease in the number or functional impairment of Tregs.^{76,107} Using the EAE model, studies have shown that adoptive transfer of polyclonal Tregs is able to attenuate the development of autoimmune diseases.⁷⁹ In contrast, disease was exacerbated when CD4+CD25+ Tregs were depleted.³ Additionally, adoptive transfer of autologous ova-specific ex vivo-expanded Tregs has been evaluated in a clinical trial for Crohn’s disease.¹⁰⁸ Although the treatment was well tolerated and showed efficacy, the results were only transient, lasting about 5 weeks, which is supported by in vivo and in vitro data suggesting that ova-Tregs have a limited survival capacity upon chronic activation.¹⁰⁸ Other difficulties with ex vivo expansion of antigen-specific Tregs include proper identification of antigens, long culture times, and overall expense.

Although the mechanism is not yet elucidated, various in vivo techniques, such as transgenic expression, liver-targeting nanoparticles, and lentivirus (LV)-mediated gene transfer, have been shown to leverage the natural ability of the liver to induce specific tolerance to an ectopically expressed autoantigen.^{46,83,84,7,12,66,64} However, even though these and other studies have provided mechanistic insight, their clinical value is currently being evaluated. Rather, an approach is needed that is translatable to the clinic and achieves robust in vivo induction of a durable Treg response, capable of reversing established autoimmune disease.

Addressing these requirements, the liver-directed AAV immunotherapy procedure presented here is based on the clinically tested AAV gene therapy platform. Overall, it provides a less complex approach for inducing antigen-specific Tregs in vivo.^{6,109} It is shown herein that a single dose of vector established a durable source of antigen needed for sustained induction and activation of autoreactive Tregs. Additionally, having engineered the vector to include the full coding sequence of MOG, it is likely to induce multiple immunodominant and sub-dominant antigen-specific Tregs, independent of MHC restrictions and without compromising long-term immune homeostasis. This is supported by previous work in a hemophilia model where an AAV vector expressing clotting factor IX was used to induce tolerance to the same transgene in multiple strains of mice.^{15,12,110,10}

As explained above, Treg immunotherapy for MS has to be capable of reversing established disease in order to be clinically feasible. The data presented clearly demonstrate that AAV.MOG immunotherapy not only prevents induction of the autoimmune disease, but more importantly clearly reverses preexisting disease if administered during early onset. However, AAV immunotherapy alone was not sufficient to fully reverse end-stage EAE disease. However, when augmented with transient immunosuppression, a potent synergistic effect was revealed that rescued mice with rapidly progressing paralysis. The use of rapamycin was specifically chosen because it induces de novo expression of FOXP3 and expands functional FOXP3+ Tregs from naive cells in vivo, while inhibiting the proliferation and trafficking of conventional CD4+ and CD8+ T cells.^{101,111,112,54} Rapamycin has also been shown to be effective at modulating EAE. Esposito et al.⁶⁹ demonstrated that continuous rapamycin monotherapy can effectively inhibit the induction and the progression of established disease; however, upon withdrawal of the drug, mice rapidly developed a relapsing-remitting form of EAE. Clearly, mitigating the inflammation in the CNS was necessary for the AAV8.MOG immunotherapy to be maximally effective.

In summary, a novel immunotherapy has been developed herein that reverses debilitating paralysis in an animal model of MS that is superior to the traditional non-specific immunosuppression therapies currently available. Accordingly, in some embodiments, this approach is used as a clinical therapy for treating MS and/or other human autoimmune diseases.

### Example 6 - Evaluation Against Challenge With Multiple Immunogenic Epitopes

Data provided herein demonstrates that a single administration of any pf the vectors described herein can provide protection against multiple epitopes, including de novo epitopes that are presented during disease progression through epitope spreading.

For example, in the C57BL/6, SJL, DB-1A and related mouse models, an experimental autoimmune encephalomyelitis (EAE) condition can be induced by injection of any one of three protein antigens—MOG, proteolipid protein (PLP), and myelin basic protein (MBP). Induction by one of these antigens produces CD4⁺ T cell-mediated inflammation in the central nervous system that serves as a relevant model for MS in humans (see, e.g., FIGS. 1, 2A, and 2B). Each of these three proteins presents different immunogenic epitopes to immune cells.

It is demonstrated herein that a single injection of the claimed AAV-MOG vector confers protection against secondary endogenous myelin epitopes (FIGS. 27A and 27B). This finding has major implications for treatment of MS, in which multiple epitope antigens are presented simultaneously in a phenomenon known as epitope spreading. This secondary protection was shown to both prevent and reverse disease conditions in EAE mice.

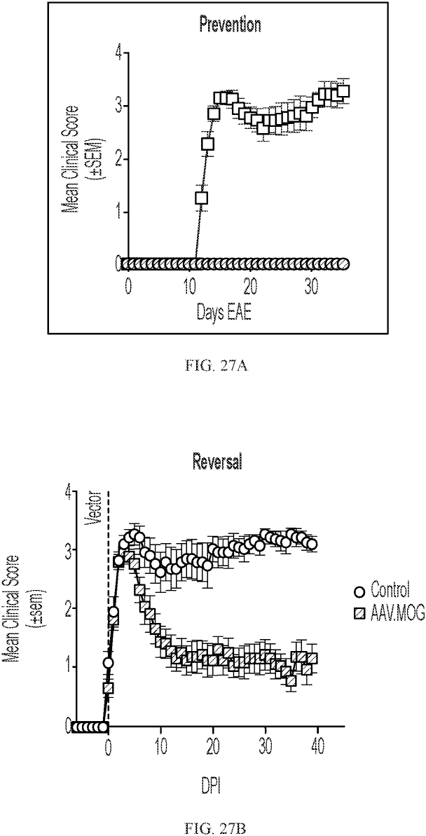

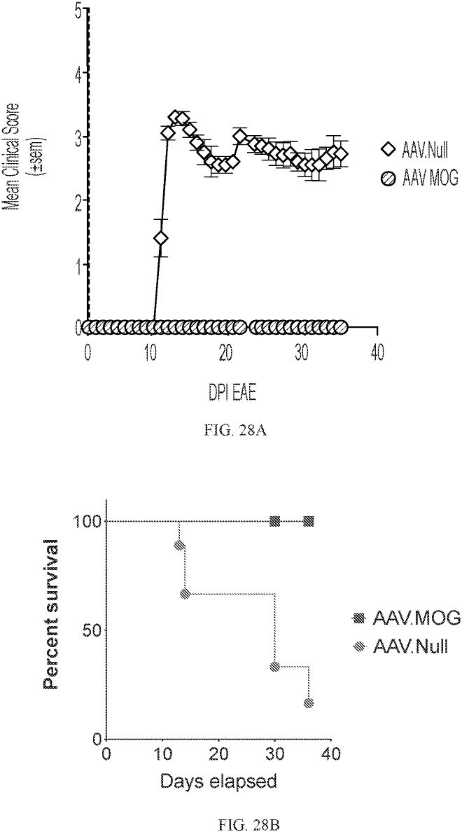

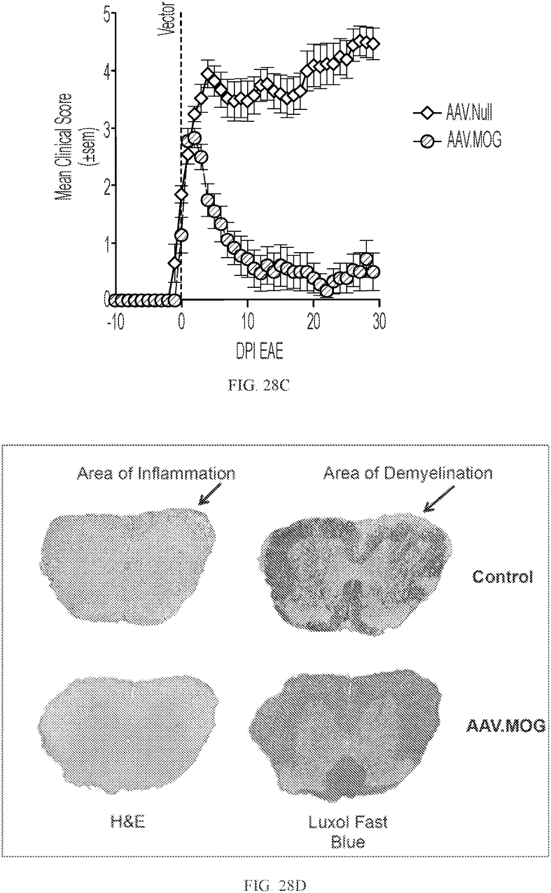

In particular, AAV8-MOG administration to mice prevented EAE (FIG. 27A) and abrogated (FIG. 27B) pre-existing EAE induced by multiple immunogenic MOG epitopes simultaneously. EAE was induced by injecting simultaneously the MOG₃₅₋₅₅ and MOG₁₁₉₋₁₃₂ epitopes suspended in complete Freund’s adjuvant (CFA). EAE symptoms were either prevented or substantially lowered in the treated group as compared to an untreated group of mice. And FIGS. 28A-28D shows that a single administration of AAV-MOG is effective at both reversing pre-existing EAE and preventing EAE onset in mice having different immunogenic epitope backgrounds. AAV8-MOG was administered to genetically diverse DBA-1 mice two weeks prior to EAE induction by the MOG₇₉₋₉₆ epitope (FIG. 28A). Vector administration prevented EAE in treated mice, while control mice developed severe EAE (FIG. 28B). AAV8-MOG was also administered to DBA-1 mice in which early EAE onset had been triggered. These mice recovered rapidly (FIG. 28C).

This data represents the first known demonstration that delivery of a Treg-inducing AAV vector is capable both of preventing and reversing MS disease phenotypes in vivo after conferring protection against multiple simultaneously-presented MOG epitopes and in mice having different immunogenic backgrounds. It establishes robust results in both chronic-progressive and relapsing-remitting models of EAE. This data indicates that the ability of the AAV-MOG vector of the claims to induce tolerance to clinically relevant epitopes in a subject is comprehensive. Additional data demonstrates that AAV-MOG administration in SJL mice did not cause any appreciable liver inflammation. The conferral of protection against multiple epitopes is contemplated herein.

Neutralization of epitope spreading remains one of the most elusive aspects of developing effective MS treatments. In 2010, it was shown that, after an initial challenge in the relapsing-remitting EAE mouse with the proteolipid protein epitope PLP₁₃₉₋₁₅₁, a second antigenic epitope, PLP₁₇₈₋₁₉₁, was revealed to immune cells during the following relapse. See Zhang et al., J. Immunol., 184:6629-6636 (2010). Subsequently, a third epitope was revealed during subsequent relapses in the EAE model against a myelin basic protein epitope. In particular, Zhang stated that, “upon transfer into SJL/J mice, [PLP₁₃₉₋₁₅₁-specific induced Tregs] undergo [antigen]-driven proliferation and are effective at suppressing induction of experimental autoimmune encephalomyelitis induced by the cognate PLP₁₃₉₋₁₅₁ peptide, but not _{PLP178-191} or a mixture of the two peptides.” Tregs specific to the PLP₁₃₉₋₁₅₁ epitope were unable to provide bystander or cross-suppression against effector T cells specific for PLP₁₇₈₋ ₁₉₁. Thus, there is substantial difficulty in reversing the EAE phenotype when EAE-induced mice are challenged with multiple immunogenic epitopes simultaneously. That a single administration of this vector could provide the efficacy of this kind without an adverse response indicates the safety and feasibility of this therapy.

Accordingly, presented herein are rAAV vectors and compsitions and particles comprising these rAAV vectors, that provide expression of an encoded therapeutic molecule in the mammal that re-establishes immune tolerance to at least two different neuroprotein epitopes. In some embodiments, these at least two different neuroproteins comprise multiple different epitopes of a single neuroprotein, such as MOG, MBP, or PLP. In particular embodiments, these at least two different neuroproteins comprise at least one epitope of a MOG protein and at least one epitope of a PLP protein. In various embodiments, the encoded therapeutic molecule is a full-length MOG, full-length MBP, or full-length PLP. In some embodiments, the encoded therapeutic molecule has a length that is less than a full-length MOG, full-length MBP, or full-length PLP.

### Example 7 - Enhanced/Synergistic Effects of Gene-Immunotherapy and Fingolimod in Models for Multiple Sclerosis

While there is no cure for Multiple Sclerosis (MS), current Disease Modifying Therapies (DMTs) focus on generalized immune suppression to slow disease progression. One such drug is Fingolimod. Fingolimod is a sphingosine analogue that modulates the sphingosine-1-phosphate (S1P) receptor and thereby alters lymphocyte migration, resulting in sequestration of lymphocytes in lymph nodes. Recent studies suggest that Tregs are not only more resistant to the fingolimod-induced sequestration from the blood and spleen to secondary lymphoid organs, but that it also induces an increased suppressive activity of Tregs. Although the mechanisms leading to an enhanced Treg activity after fingolimod treatment is currently not well understood, previous studies have demonstrated that S1P has the ability to alter the activity of Tregs by enhancing IL-10 production and that S1P was found to be required for the optimal activity of CD4+/CD25+ T-regulatory cells.

To increase the specificity of the otherwise generalized immune suppression of DMTs, a novel gene immunotherapy has been developed that can selectively induce antigen specific regulatory T cells (Tregs). This in turn can modulate the adverse immune response against specific myelin proteins in autoimmune diseases. This novel approach has been demonstrated to not only prevent, but also reverse Experimental Autoimmune Encephalomyelitis (EAE), an autoimmune animal model of Multiple Sclerosis (MS) using an Adeno-associated virus (AAV) vector expressing a specific neuropeptide.

When considering the development of a clinical trial, patients are likely to be receiving a DMT or will start a treatment protocol as the established standard of care. The prevailing goal of gene-immunotherapy is to restore immune tolerance so that long-term continuous use of a DMT such as fingolimod is unnecessary. As such, it is equally vital to ensure that the medical treatment currently being administered to the patient does not adversely affect the ability of the gene-immunotherapy to re-establish and maintain tolerance. Thus, it was necessary to assess the ability of the gene-immunotherapy disclosed herein (e.g., the AAV vector expressing myelin oligodendrocyte glycoprotein (AAV.MOG)) to induce immunological tolerance while a patient is currently being treated with the drug Fingolimod, an established immunosuppressant used in the treatment of MS patients.

Using 8-week-old Female C57BL/6 mice, EAE was induced with a myelin oligodendrocyte glycoprotein epitope (MOG₃₅₋₅₅). At first sign of clinical disease, mice were injected with a hepatocyte directed viral vector encoding MOG (e.g., AAV.MOG vector) and began daily administration of fingolimod via oral gavage until day 24. Control mice were treated with fingolimod only, without the administration of vector or with Null vector. Initially, all mice treated with fingolimod had recovered from EAE symptoms. At day 24, fingolimod treatment was discontinued, except for the half of the control group receiving treatment which continued receiving fingolimod.

By day 20 post treatment, all treated mice had recovered. However, after treatment was stopped mice that were only receiving fingolimod relapsed and developed severe EAE (FIG. 29A). Whereas mice treated with AAV.MOG and fingolimod remained nearly disease free. The data herein showing concomitant administration of vector and fingolimod demonstrates a synergistic effect that results in a significant long-term reversal of disease even upon withdrawal of DMT treatment.

Within days, non-MOG vectored mice treated with fingolimod relapsed and developed severe EAE. Whereas mice treated with MOG vector and Fingolimod remained nearly disease free (FIG. 29B). Furthermore, the data demonstrates a synergistic effect of vector and fingolimod that results in a significant long-term reversal of disease upon withdrawal of DMT treatment (FIG. 29C). The cessation of Fingolimod treatment in MS patients has been associated with cases of severe rebound syndrome leading to severe relapses or high MRI activity.

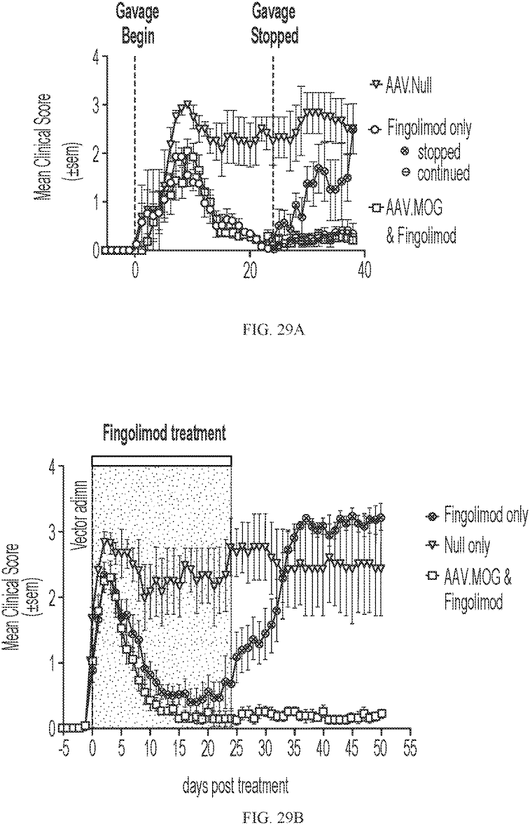

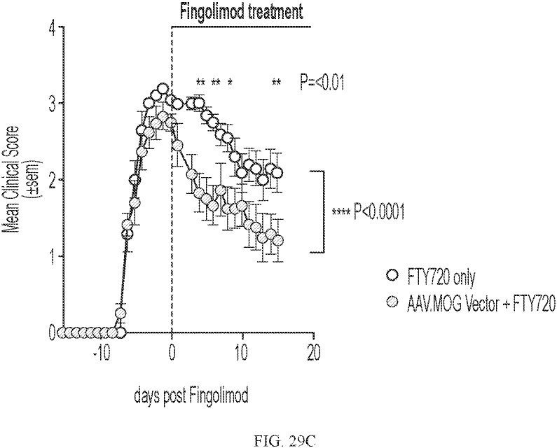

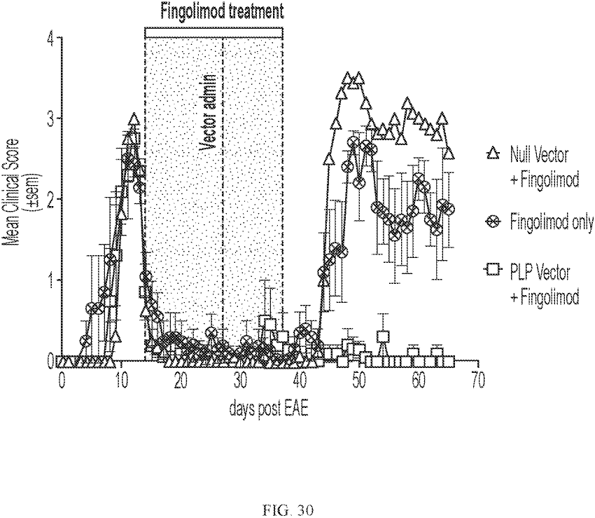

In another experiment, the use of Fingolimod and gene-immunotherapy was tested in the relapsing-remitting model of MS using proteolipid protein (PLP)-induced EAE in cohorts of SJL mice. Here, Fingolimod treatment was administered following the initial remittance of disease. Therapeutic vector expressing PLP transgene was subsequently given 2-weeks later, and Fingolimod treatment continued for another 2-weeks. As before, shortly after Fingolimod treatment was stopped, non-therapeutically vectored mice quickly developed severe EAE disease, whereas treated mice remained disease free (FIG. 30).

In sum, simultaneous treatment using directed gene-immunotherapy (e.g., one or more rAAV vectors encoding one or more MOG, PLP, and/or MBP proteins, for example AAV8-MOG) and the DMT fingolimod shows no apparent inhibitory effect on the function of the vector. In fact, it appears that, when given as a combination, the two may work synergistically to reverse disease progression and increase the effectiveness of gene-immunotherapy. The AAV gene-immunotherapy disclosed herein has significant clinical relevance as it restores a persistent and continuous immune tolerance such that long-term continuous DMT may be unnecessary for MS patients.

### Example 8 - Synergistic Effects of Gene-Immunotherapy and Prednisolone in Models for Multiple Sclerosis

Another DMT evaluated in combination therapies with the disclosed vectors is prednisolone. Prednisolone, a glucocorticoid alters polymorphonuclear leukocyte migration, resulting in sequestration in lymph nodes, and reduction of inflammation. Prednisone, another DMT being evaluated in these combination therapies, is a prodrug to prednisolone, and specifically is converted to prednisolone in the liver. Prednisone and prednisolone are capable of binding to glucocorticoid receptors (GCRs). Tregs may be resistant to the glucocorticoidinduced sequestration from the blood and spleen to secondary lymphoid organs. As with fingolimod, prednisolone an established immunosuppressant used in the treatment of patients of MS and other autoimmune diseases. Recent studies have shown that subjects in clinical trials for other AAV therapies that begin to have an adverse immune response to the AAV are often given prednisolone to prevent failure of the gene therapy.

As with fingolimod, it is vital to ensure that the medical treatment currently being administered to the patient does not adversely affect the ability of the gene-immunotherapy to re-establish and maintain tolerance. Thus, it was necessary to assess the ability of the AAV vectors disclosed herein (e.g., the AAV vector expressing myelin oligodendrocyte glycoprotein (AAV.MOG)) to induce immunological tolerance while a patient is currently being treated with the drug prednisolone.

Using 8-week-old Female C57BL/6 mice, EAE was induced with a myelin oligodendrocyte glycoprotein epitope (MOG₃₅₋₅₅) in CFA. As mice developed disease (MCS 2.0± 0.5), mice were injected with a hepatocyte-directed viral vector encoding MOG (e.g., AAV.MOG vector) and began daily administration of prednisolone (PRDL) via oral gavage (10 mg/kg animal) every 24 hours until day 7. Control mice were treated with prednisolone only, without the administration of vector or with Null vector. At day 7, prednisolone treatment was discontinued, except for the half of the control group receiving treatment which continued receiving prednisolone.

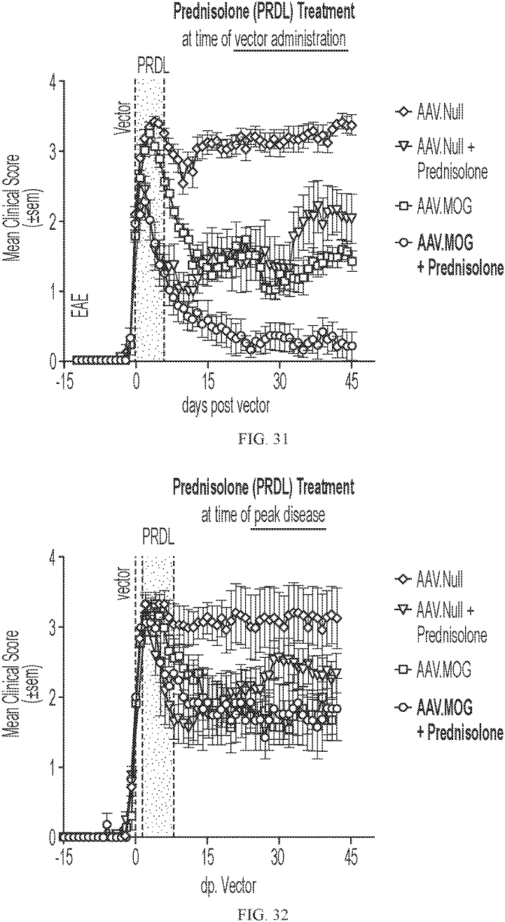

As shown in FIG. 31, AAV.MOG +Prednisolone was able to maintain EAE suppression. Maintenance of suppression was surpsingly superior to AAV.MOG alone, following withdrawal of PRDL.

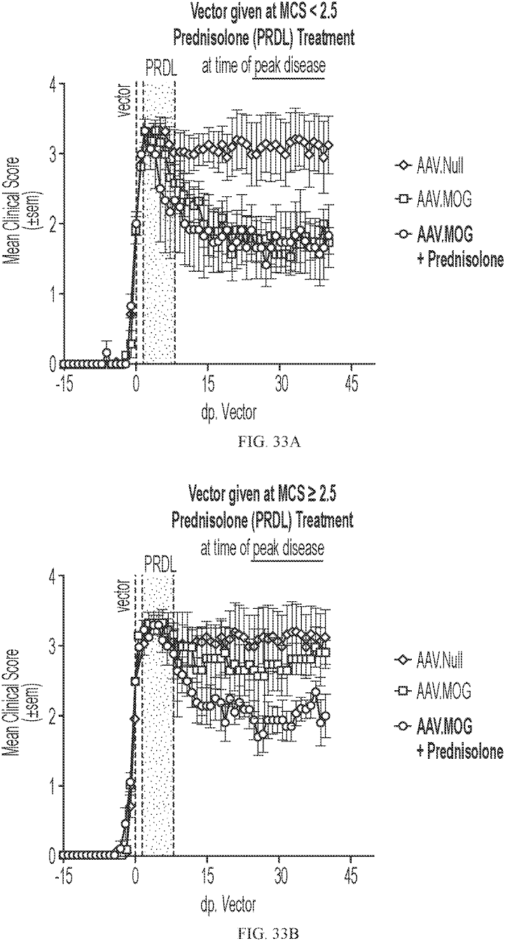

An experiment was performed to assess the effect of supplementing rAAV therapy with PRDL in high clinical score mice. C57BL/6 mice were administered AAV.MOG or AAV.Null control at disease onset, or a mean clinical score of 2.0±0.5. Subsequently, at the peak of disease, or an MCS ≥ 3.0, mice were administered oral gavage of PRDL (10 mg/kg). As shown in FIG. 32, AAV.MOG+Prednisolone provided EAE suppression. In a similar experiment, mice were administered AAV.MOG or AAV.Null control at early disease onset (before disease severity), or an MCS of <2.5. Subsequently, at an MCS ≥ 3.0, mice were administered oral gavage of PRDL (10 mg/kg). The results shown in in FIG. 33A corroborate the earlier results that if AAV vector was initially adminstered to subjects at a clinical score less than 2.5 and the PRDL administered at peak MCS of 3.0, the PRDL did not interfere with induction of tolerance.

In an experiment in which the order of administrations was essentially reversed, C57BL/6 mice received AAV.MOG or AAV.Null after EAE developed to an MCS of 2.5 or higher. At the peak of disease (MCS ≥3.0), mice were started on oral gavage of PRDL as before. AAV.MOG+Prednisolone maintained suppression after withdrawal of PRDL (as shown in FIG. 33B). Collectively, these results indicate when vector was administered during early disease onset, PDRL did not interfere with, nor supplement, the ability of AAV.MOG to suppress EAE. However, if AAV.MOG vector was withheld until more severe developed, the combination of PDRL with vector was much more effective than AAV.MOG alone.

The data herein showing concomitant administration of vector and prednisolone demonstrates a synergistic effect that results in long-term reversal of disease even upon withdrawal of prednisolone treatment. In sum, simultaneous treatment using directed gene-immunotherapy (e.g., one or more rAAV vectors encoding one or more MOG, PLP, and/or MBP proteins, for example AAV8-MOG) and the DMT prednisolone shows no apparent inhibitory effect on the function of the vector. In fact, it appears that, when given as a combination, the two therapeutics may work synergistically to reverse disease progression and increase the effectiveness of gene-immunotherapy.

### REFERENCES

The following references, to the extent that they provide exemplary procedural or other details supplementary to those set forth herein, are specifically incorporated herein by reference:

| | |
|---|---|
| 1. | Kohm AP, Carpentier PA, Anger HA, Miller SD. Cutting edge: CD4+CD25+ regulatory T cells suppress antigen-specific autoreactive immune responses and central nervous system inflammation during active experimental autoimmune encephalomyelitis. Journal of Immunology (Baltimore, MD). 169(9):4712-4716 (2002). |
| 2. | Zhang G-X, Yu S, Gran B, Li J, Calida D, Ventura E, Chen X, Rostami A. T cell and antibody responses in remitting-relapsing experimental autoimmune encephalomyelitis in (C57BL/6×SJL) Fl mice. Journal of Neuroimmunology. 2004;148(1-2):1-10. |
| 3. | McGeachy MJ, Stephens LA, Anderton SM. Natural recovery and protection from autoimmune encephalomyelitis: contribution of CD4+CD25+ regulatory cells within the central nervous system. Journal of Immunology (Baltimore, MD), 2005;175(5):3025-3032. |
| 4. | Paust S, Cantor H. Regulatory T cells and autoimmune disease. Immunological reviews. 2005;204:195-207. |
| 5. | Marusić S, Tonegawa S. Tolerance induction and autoimmune encephalomyelitis amelioration after administration of myelin basic protein-derived peptide. Journal of Experimental Medicine. 1997;186(4):507-515. |
| 6. | LoDuca PA, Hoffman BE, Herzog RW. Hepatic gene transfer as a means of tolerance induction to transgene products. Current gene therapy. 2009;9(2): 104-14. |
| 7. | Hoffman B, Dobrzynski E, Wang L, Hirao L, Mingozzi F, Cao O, Herzog RW. Muscle as a Target for Supplementary Factor IX Gene Transfer2007;18(7):603-13. |
| 8. | Dobrzynski E, Fitzgerald JC, Cao O, Mingozzi F, Wang L, Herzog RW. Prevention of cytotoxic T lymphocyte responses to factor IX-expressing hepatocytes by gene transferinduced regulatory T cells. Proceedings of the National Academy of Sciences of the United States of America. 2006;103(12):4592-7. |
| 9. | Dobrzynski E, Herzog RW. Tolerance induction by viral in vivo gene transfer. Clinical medicine & research. 2005;3(4):234-40. |
| 10. | Cooper M, Nayak S, Hoffman BE, Terhorst C, Cao O, Herzog RW. Improved induction of immune tolerance to factor IX by hepatic AAV-8 gene transfer. Human gene therapy. 2009;20(7):767-76. |
| 11. | Cao O, Furlan-Freguia C, Arruda VR, Herzog RW. Emerging role of regulatory T cells in gene transfer. Curr Gene Ther. 2007;7(5):381-90. |
| 12. | Hoffman BE, Martino AT, Sack BK, Cao O, Liao G, Terhorst C, Herzog RW. Nonredundant roles of IL-10 and TGF-beta in suppression of immune responses to hepatic AAV-factor IX gene transfer. Molecular therapy : the journal of the American Society of Gene Therapy. 2011;19(7):1263-72. |
| 13. | Mingozzi F, Liu YL, Dobrzynski E, Kaufhold A, Liu JH, Wang Y, Arruda VR, High KA, Herzog RW. Induction of immune tolerance to coagulation factor IX antigen by in vivo hepatic gene transfer. The Journal of clinical investigation. 2003;111(9):1347-56. doi: 10.1172/JCI16887. PMID: 12727926. |
| 14. | Faust SM, Bell P, Zhu Y, Sanmiguel J, Wilson JM. The role of apoptosis in immune hyporesponsiveness following AAV8 liver gene transfer. Molecular therapy : the journal of the American Society of Gene Therapy. 2013;21(12):2227-35. |
| 15. | Markusic DM, Hoffman BE, Perrin GQ, Nayak S, Wang X, LoDuca PA, High KA, Herzog RW. Effective gene therapy for haemophilic mice with pathogenic factor IX antibodies. EMBO Molecular Medicine. 2013:1-12. |
| 16. | Annoni A, Brown BD, Cantore A, Sergi LS, Naldini L, Roncarolo MG. In vivo delivery of a microRNA-regulated transgene induces antigen-specific regulatory T cells and promotes immunologic tolerance. Blood. 2009;114(25):5152-61. |
| 17. | Breous E, Somanathan S, Vandenberghe LH, Wilson JM. Hepatic regulatory T cells and Kupffer cells are crucial mediators of systemic T cell tolerance to antigens targeting murine liver. Hepatology. 2009;50(2):612-21. |
| 18. | Matrai J, Cantore A, Bartholomae CC, Annoni A, Wang W, Acosta-Sanchez A, Samara-Kuko E, De Waele L, Ma L, Genovese P, Damo M, Arens A, Goudy K, Nichols TC, von Kalle C, MK LC, Roncarolo MG, Schmidt M, Vandendriessche T, Naldini L. Hepatocyte-targeted expression by integrase-defective lentiviral vectors induces antigen-specific tolerance in mice with low genotoxic risk. Hepatology. 2011;53(5):1696-707. |
| 19. | Mingozzi F, Hasbrouck NC, Basner-Tschakarjan E, Edmonson SA, Hui DJ, Sabatino DE, Zhou S, Wright JF, Jiang H, Pierce GF, Arruda VR, High KA. Modulation of tolerance to the transgene product in a nonhuman primate model of AAV-mediated gene transfer to liver. Blood. 2007;110(7):2334-41. |
| 20. | Sun B, Kulis MD, Young SP, Hobeika AC, Li S, Bird A, Zhang H, Li Y, Clay TM, Burks W, Kishnani PS, Koeberl DD. Immunomodulatory gene therapy prevents antibody formation and lethal hypersensitivity reactions in murine pompe disease. Molecular therapy : the journal of the American Society of Gene Therapy. 2010;18(2):353-360. |
| 21. | Miao CH, Harmeling BR, Ziegler SF, Yen BC, Torgerson T, Chen L, Yau RJ, Peng B, Thompson AR, Ochs HD, Rawlings DJ. CD4+FOXP3+ regulatory T cells confer long-term regulation of factor VIII-specific immune responses in plasmid-mediated gene therapy-treated hemophilia mice. Blood. 2009;114(19):4034-44. |
| 22. | Brown BD, Cantore A, Annoni A, Sergi LS, Lombardo A, Della Valle P, D′Angelo A, Naldini L. A microRNA-regulated lentiviral vector mediates stable correction of hemophilia B mice. Blood. 2007;110(13):4144-52. |
| 23. | Brown BD, Venneri MA, Zingale A, Sergi L, Naldini L. Endogenous microRNA regulation suppresses transgene expression in hematopoietic lineages and enables stable gene transfer. Nature medicine. 2006;12(5):585-91. |
| 24. | Cerullo V, McCormack W, Seiler M, Mane V, Cela R, Clarke C, Rodgers JR, Lee B. Antigen-specific tolerance of human alpha1-antitrypsin induced by helper-dependent adenovirus. Human gene therapy. 2007;18(12):1215-24. |
| 25. | Follenzi A, Battaglia M, Lombardo A, Annoni A, Roncarolo MG, Naldini L. Targeting lentiviral vector expression to hepatocytes limits transgene-specific immune response and establishes long-term expression of human antihemophilic factor IX in mice. Blood. 2004;103(10):3700-9. |
| 26. | Franco LM, Sun B, Yang X, Bird A, Zhang H, Schneider A, Brown T, Young SP, Clay TM, Amalfitano A, Chen YT, Koeberl DD. Evasion of immune responses to introduced human acid alpha-glucosidase by liver-restricted expression in glycogen storage disease type II. Molecular therapy: the journal of the American Society of Gene Therapy. 2005;12(5):876-84. |
| 27. | Koeberl DD, Kishnani PS. Immunomodulatory gene therapy in lysosomal storage disorders. Curr Gene Ther. 2009;9(6):503-10. |
| 28. | Luth S, Huber S, Schramm C, Buch T, Zander S, Stadelmann C, Bruck W, Wraith DC, Herkel J, Lohse AW. Ectopic expression of neural autoantigen in mouse liver suppresses experimental autoimmune neuroinflammation by inducing antigen-specific Tregs. The Journal of clinical investigation. 2008;118(10):3403-10. |
| 29. | Matsui H, Hegadorn C, Ozelo M, Burnett E, Tuttle A, Labelle A, McCray PB, Jr., Naldini L, Brown B, Hough C, Lillicrap D. A microRNA-regulated and GP64-pseudotyped lentiviral vector mediates stable expression of FVIII in a murine model of Hemophilia A. Molecular therapy: the journal of the American Society of Gene Therapy. 2011;19(4):723-30. |
| 30. | Matsui H, Shibata M, Brown B, Labelle A, Hegadorn C, Andrews C, Chuah M, VandenDriessche T, Miao CH, Hough C, Lillicrap D. A murine model for induction of long-term immunologic tolerance to factor VIII does not require persistent detectable levels of plasma factor VIII and involves contributions from Foxp3+ T regulatory cells. Blood. 2009;114(3):677-85. |
| 31. | McEachern KA, Nietupski JB, Chuang WL, Armentano D, Johnson J, Hutto E, Grabowski GA, Cheng SH, Marshall J. AAV8-mediated expression of glucocerebrosidase ameliorates the storage pathology in the visceral organs of a mouse model of Gaucher disease. Journal of Gene Medicine, 2006;8(6):719-29. |
| 32. | Nietupski JB, Hurlbut GD, Ziegler RJ, Chu Q, Hodges BL, Ashe KM, Bree M, Cheng SH, Gregory RJ, Marshall J, Scheule RK. Systemic administration of AAV8-alpha-galactosidase A induces humoral tolerance in nonhuman primates despite low hepatic expression. Molecular therapy : the journal of the American Society of Gene Therapy. 2011;19(11):1999-2011. |
| 33. | Passini MA, Bu J, Fidler JA, Ziegler RJ, Foley JW, Dodge JC, Yang WW, Clarke J, Taksir TV, Griffiths DA, Zhao MA, O′Riordan CR, Schuchman EH, Shihabuddin LS, Cheng SH. |
| | Combination brain and systemic injections of AAV provide maximal functional and survival benefits in the Niemann-Pick mouse. Proceedings of the National Academy of Sciences of the United States of America. 2007;104(22):9505-10. |
| 34. | Sharland A, Logan GJ, Bishop A, Alexander IE. Liver-directed gene expression using recombinant AAV 2/8 vectors--a tolerogenic strategy for gene delivery? Discovery medicine. 2010;9(49):519-27. |
| 35. | Somanathan S, Breous E, Bell P, Wilson JM. AAV vectors avoid inflammatory signals necessary to render transduced hepatocyte targets for destructive T cells. Molecular therapy : the journal of the American Society of Gene Therapy. 2010;18(5):977-82. |
| 36. | Sun B, Bird A, Young SP, Kishnani PS, Chen YT, Koeberl DD. Enhanced response to enzyme replacement therapy in Pompe disease after the induction of immune tolerance. American journal of human genetics. 2007;81(5):1042-9. |
| 37. | Ziegler RJ, Lonning SM, Armentano D, Li C, Souza DW, Cherry M, Ford C, Barbon CM, Desnick RJ, Gao G, Wilson JM, Peluso R, Godwin S, Carter BJ, Gregory RJ, Wadsworth SC, Cheng SH. AAV2 vector harboring a liver-restricted promoter facilitates sustained expression of therapeutic levels of alpha-galactosidase A and the induction of immune tolerance in Fabry mice. Molecular therapy : the journal of the American Society of Gene Therapy. 2004;9(2):231-40. |
| 38. | Moses H, Picone MA, Smith V. An In-Depth Overview: Consensus Medical Communications; 2012. |
| 39. | Gonsette RE. Self-tolerance in multiple sclerosis. Acta neurologica Belgica. 2012;112(2): 133-40 |
| 40. | Viglietta V, Baecher-Allan C, Weiner HL, Hafler DA. Loss of functional suppression by CD4+CD25+ regulatory T cells in patients with multiple sclerosis. J Exp Med. 2004; 199(7) :971-9 |
| 41. | Zozulya AL, Wiendl H. The role of regulatory T cells in multiple sclerosis. Nature Clinical Practice Neurology. 2008;4(7):384-98. |
| 42. | Buckner JH. Mechanisms of impaired regulation by CD4(+)CD25(+)FOXP3(+) regulatory T cells in human autoimmune diseases. Nature Reviews Immunology. 2010;10(12):849-59. doi: 10.1038/nri2889. |
| 43. | Buc M. Role of Regulatory T Cells in Pathogenesis and Biological Therapy of Multiple Sclerosis. Mediators of Inflammation. 2013;2013(6):1-11. |
| 44. | Costantino CM, Baecher-Allan C, Hafler DA. Multiple Sclerosis and Regulatory T Cells. Journal of Clinical Immunology. 2008;28(6):697-706. |
| 45. | Hoffman BE, Herzog RW. Coaxing the liver into preventing autoimmune disease in the brain. The Journal of clinical investigation. 2008;118(10):3271-3. |
| 46. | Lüth S, Huber S, Schramm C, Buch T, Zander S, Stadelmann C, Brück W, Wraith DC, Herkel J, Lohse AW. Ectopic expression of neural autoantigen in mouse liver suppresses experimental autoimmune neuroinflammation by inducing antigen-specific Tregs. The Journal of clinical investigation. 2008;118(10):3403-10. |
| 47. | Haas J, Korporal M, Balint B, Fritzsching B, Schwarz A, Wildemann B. Glatiramer acetate improves regulatory T-cell function by expansion of naive CD4(+)CD25(+)FOXP3(+)CD31(+) T-cells in patients with multiple sclerosis. J Neuroimmunol. 2009;216(1-2):113-7. |
| 48. | Nathwani AC, Tuddenham EG, Rangarajan S, Rosales C, McIntosh J, Linch DC, Chowdary P, Riddell A, Pie AJ, Harrington C, O′Beirne J, Smith K, Pasi J, Glader B, Rustagi P, Ng CY, Kay MA, Zhou J, Spence Y, Morton CL, Allay J, Coleman J, Sleep S, Cunningham JM, Srivastava D, Basner-Tschakarjan E, Mingozzi F, High KA, Gray JT, Reiss UM, Nienhuis AW, Davidoff AM. Adenovirus-associated virus vector-mediated gene transfer in hemophilia B. The New England journal of medicine. 2011;365(25):2357-65. |
| 49. | Herzog RW, Cao O, Srivastava A. Two decades of clinical gene therapy--success is finally mounting. Discovery medicine. 2010;9(45):105-11. Epub 2010/03/03. |
| 50. | Mingozzi F, High KA. Therapeutic in vivo gene transfer for genetic disease using AAV: progress and challenges. Nat Rev Genet. 2011;12(5):341-55. |
| 51. | Maclaren RE, Groppe M, Barnard AR, Cottriall CL, Tolmachova T, Seymour L, Clark KR, During MJ, Cremers FP, Black GC, Lotery AJ, Downes SM, Webster AR, Seabra MC. Retinal gene therapy in patients with choroideremia: initial findings from a phase ½ clinical trial. Lancet. 2014. Epub 2014/01/21. |
| 52. | Bryant LM, Christopher DM, Giles AR, Hinderer C, Rodriguez JL, Smith JB, Traxler EA, Tycko J, Wojno AP, Wilson JM. Lessons learned from the clinical development and market authorization of Glybera. Human gene therapy Clinical development. 2013;24(2):55-64. |
| 53. | Byrne BJ, Falk DJ, Pacak CA, Nayak S, Herzog RW, Elder ME, Collins SW, Conlon TJ, Clement N, Cleaver BD, Cloutier DA, Porvasnik SL, Islam S, Elmallah MK, Martin A, Smith BK, Fuller DD, Lawson LA, Mah CS. Pompe disease gene therapy. Human molecular genetics. 2011;20(R1):R61-R68. |
| 54. | Moghimi B, Sack BK, Nayak S, Markusic DM, Mah CS, Herzog RW. Induction of tolerance to factor VIII by transient co-administration with rapamycin. Journal of thrombosis and haemostasis: JTH. 2011;9(8):1524-1533. |
| 55. | Nayak S, Cao O, Hoffman BE, Cooper M, Zhou S, Atkinson MA, Herzog RW. Prophylactic immune tolerance induced by changing the ratio of antigen-specific effector to regulatory T cells. Journal of thrombosis and haemostasis : JTH. 2009;7(9):1523-1532. |
| 56. | Nayak S, Sarkar D, Perrin GQ, Moghimi B, Hoffman BE, Zhou S, Byrne BJ, Herzog RW. Prevention and Reversal of Antibody Responses Against Factor IX in Gene Therapy for Hemophilia B. Frontiers in Microbiology. 2011;2. |
| 57. | Martino AT, Nayak S, Hoffman BE, Cooper M, Liao G, Markusic DM, Byrne BJ, Terhorst C, Herzog RW. Tolerance induction to cytoplasmic beta-galactosidase by hepatic AAV gene transfer: implications for antigen presentation and immunotoxicity. PloS one. 2009;4(8):e6376. |
| 58. | Lalive PH, Molnarfi N, Benkhoucha M, Weber MS, Santiago-Raber ML. Antibody response in MOG(35-55) induced EAE. J Neuroimmunol. 2011;240-241:28-33. doi: 10.1016/j.jneuroim.2011.09.005. |
| 59. | Mann MK, Ray A, Basu S, Karp CL, Dittel BN. Pathogenic and regulatory roles for B cells in experimental autoimmune encephalomyelitis. Autoimmunity. 2012;45(5):388-399. |
| 60. | Miller SD, Karpus WJ, Davidson TS. Experimental autoimmune encephalomyelitis in the mouse. Current protocols in immunology / edited by John E Coligan [et al]. 2010;Chapter 15:Unit 15.1. |
| 61. | Ko HJ, Chung JY, Nasa Z, Chan J, Siatskas C, Toh BH, Alderuccio F. Targeting MOG expression to dendritic cells delays onset of experimental autoimmune disease. Autoimmunity. 2011;44(3):177-87. |
| 62. | Dobrzynski E, Mingozzi F, Liu YL, Bendo E, Cao O, Wang L, Herzog RW. Induction of antigen-specific CD4+ T-cell anergy and deletion by in vivo viral gene transfer. Blood. 2004;104(4):969-77. |
| 63. | Fissolo N, Costa C, Nurtdinov RN, Bustamante MF, Llombart V, Mansilla MJ, Espejo C, Montalban X, Comabella M. Treatment with MOG-DNA vaccines induces CD4+CD25+FoxP3+ regulatory T cells and up-regulates genes with neuroprotective functions in experimental autoimmune encephalomyelitis. Journal of neuroinflammation. 2012;9:139. |
| 64. | O′Connor RA, Anderton SM. Foxp3+ regulatory T cells in the control of experimental CNS autoimmune disease. J Neuroimmunol. 2008;193(1-2):1-11. |
| 65. | Tang Q, Henriksen KJ, Bi M, Finger EB, Szot G, Ye J, Masteller EL, McDevitt H, Bonyhadi M, Bluestone JA. In vitro-expanded antigen-specific regulatory T cells suppress autoimmune diabetes. J Exp Med. 2004;199(11):1455-65. |
| 66. | Chen X, Oppenheim JJ, Winkler-Pickett RT, Ortaldo JR, Howard OM. Glucocorticoid amplifies IL-2-dependent expansion of functional FoxP3(+)CD4(+)CD25(+) T regulatory cells in vivo and enhances their capacity to suppress EAE. European journal of immunology. 2006;36(8):2139-49. |
| 67. | Oleszak EL, *Hoffman BE, Chang JR, Zaczynska E, Gaughan J, Katsetos CD, Platsoucas CD, Harvey N. Apoptosis of infiltrating T cells in the central nervous system of mice infected with Theiler’s murine encephalomyelitis virus. Virology. 2003;315(1):110-23. Epub 2003/11/01. |
| 68. | Selvaraj RK, Geiger TL. Mitigation of experimental allergic encephalomyelitis by TGF-beta induced Foxp3+ regulatory T lymphocytes through the induction of anergy and infectious tolerance. Journal of immunology. 2008;180(5):2830-8. |
| 69. | Esposito M, Ruffini F, Bellone M, Gagliani N, Battaglia M, Martino G, Furlan R. Rapamycin inhibits relapsing experimental autoimmune encephalomyelitis by both effector and regulatory T cells modulation. J. Neuroimmunol., 2010;220(1-2):52-63. |
| 70. | Bensinger SJ, Walsh PT, Zhang J, Carroll M, Parsons R, Rathmell JC, Thompson CB, Burchill MA, Farrar MA, Turka LA. Distinct IL-2 receptor signaling pattern in CD4+CD25+ regulatory T cells. Journal of immunology. 2004;172(9):5287-96. |
| 71. | O′Connor RA, Malpass KH, Anderton SM. The inflamed central nervous system drives the activation and rapid proliferation of Foxp3+ regulatory T cells. Journal of immunology. 2007;179(2):958-66. |
| 72. | Korn T, Reddy J, Gao W, Bettelli E, Awasthi A, Petersen TR, Backstrom BT, Sobel RA, Wucherpfennig KW, Strom TB, Oukka M, Kuchroo VK. Myelin-specific regulatory T cells accumulate in the CNS but fail to control autoimmune inflammation. Nature medicine. 2007;13(4):423-31. |
| 73. | Niemeyer GP, Herzog RW, Mount J, Arruda VR, Tillson DM, Hathcock J, van Ginkel FW, High KA, Lothrop CD, Jr. Long-term correction of inhibitor-prone hemophilia B dogs treated with liver-directed AAV2-mediated factor IX gene therapy. Blood. 2009 113(4):797-806. |
| 74. | Dalla Libera D., Di Mitri D., Bergami A., Centonze D., Gasperini C., Grasso M.G., Galgani S., Martinelli V., Comi G., Avolio C. T regulatory cells are markers of disease activity in multiple sclerosis patients. PLoS ONE. 2011;6:e21386. |
| 75. | Carbone F., De Rosa V., Carrieri P.B., Montella S., Bruzzese D., Porcellini A., Procaccini C., La Cava A., Matarese G. Regulatory T cell proliferative potential is impaired in human autoimmune disease. Nat. Med. 2014;20:69-74. |
| 76. | Vila J., Isaacs J.D., Anderson A.E. Regulatory T cells and autoimmunity. Curr. Opin. Hematol. 2009;16:274-279. |
| 77. | MacDonald K.G., Hoeppli R.E., Huang Q., Gillies J., Luciani D.S., Orban P.C., Broady R., Levings M.K. Alloantigen-specific regulatory T cells generated with a chimeric antigen receptor. J. Clin. Invest. 2016;126:1413-1424. |
| 78. | Rossetti M., Spreafico R., Saidin S., Chua C., Moshref M., Leong J.Y., Tan Y.K., Thumboo J., van Loosdregt J., Albani S. Ex vivo-expanded but not in vitro-induced human regulatory T cells are candidates for cell therapy in autoimmune diseases thanks to stable demethylation of the FOXP3 regulatory T cell-specific demethylated region. J. Immunol. 2015;194:113-124. |
| 79. | Beyersdorf N., Gaupp S., Balbach K., Schmidt J., Toyka K.V., Lin C.H., Hanke T., Hünig T., Kerkau T., Gold R. Selective targeting of regulatory T cells with CD28 superagonists allows effective therapy of experimental autoimmune encephalomyelitis. J. Exp. Med. 2005;202:445-455. |
| 80. | Thomas H.R., Gitelman S.E. Altering the course of type 1 diabetes: an update on prevention and new-onset clinical trials. Pediatr. Diabetes. 2013;14:311-321. |
| 81. | Trzonkowski P., Bieniaszewska M., Juścińska J., Dobyszuk A., Krzystyniak A., Marek N., Myśliwska J., Hellmann A. First-in-man clinical results of the treatment of patients with graft versus host disease with human ex vivo expanded CD4+CD25+CD127- T regulatory cells. Clin. Immunol. 2009;133:22-26. |
| 82. | Safinia N., Sagoo P., Lechler R., Lombardi G. Adoptive regulatory T cell therapy: challenges in clinical transplantation. Curr. Opin. Organ Transplant. 2010;15:427-434. |
| 83. | Akbarpour M., Goudy K.S., Cantore A., Russo F., Sanvito F., Naldini L., Annoni A., Roncarolo M.G. Insulin B chain 9-23 gene transfer to hepatocytes protects from type 1 diabetes by inducing Ag-specific FoxP3+ Tregs. Sci. Transl. Med. 2015;7:289ra81. |
| 84. | Sack B.K., Herzog R.W., Terhorst C., Markusic D.M. Development of gene transfer for induction of antigen-specific tolerance. Mol. Ther. Methods Clin. Dev. 2014;1:14013. |
| 85. | Zolotukhin S., Potter M., Zolotukhin I., Sakai Y., Loiler S., Fraites T.J., Jr., Chiodo V.A., Phillipsberg T., Muzyczka N., Hauswirth W.W. Production and purification of serotype 1, 2, and 5 recombinant adeno-associated viral vectors. Methods. 2002;28:158-167. |
| 86. | Martino A.T., Suzuki M., Markusic D.M., Zolotukhin I., Ryals R.C., Moghimi B., Ertl H.C., Muruve D.A., Lee B., Herzog R.W. The genome of self-complementary adeno-associated viral vectors increases Toll-like receptor 9-dependent innate immune responses in the liver. Blood. 2011;117:6459-6468. |
| 87. | Kerlero de Rosbo N., Milo R., Lees M.B., Burger D., Bernard C.C., Ben-Nun A. Reactivity to myelin antigens in multiple sclerosis. Peripheral blood lymphocytes respond predominantly to myelin oligodendrocyte glycoprotein. J. Clin. Invest. 1993;92:2602-2608. |
| 88. | Varrin-Doyer M., Shetty A., Spencer C.M., Schulze-Topphoff U., Weber M.S., Bernard C.C., Forsthuber T., Cree B.A., Slavin A.J., Zamvil S.S. MOG transmembrane and cytoplasmic domains contain highly stimulatory T-cell epitopes in MS. Neurol. Neuroimmunol. Neuroinflamm. 2014;1:e20. |
| 89. | Cao O., Dobrzynski E., Wang L., Nayak S., Mingle B., Terhorst C., Herzog R.W. Induction and role of regulatory CD4+CD25+ T cells in tolerance to the transgene product following hepatic in vivo gene transfer. Blood. 2007;110:1132-1140. |
| 90. | Mingozzi F., High K.A. Immune responses to AAV vectors: overcoming barriers to successful gene therapy. Blood. 2013;122:23-36. |
| 91. | Kim W.R., Flamm S.L., Di Bisceglie A.M., Bodenheimer H.C., Public Policy Committee of the American Association for the Study of Liver Disease Serum activity of alanine aminotransferase (ALT) as an indicator of health and disease. Hepatology. 2008;47:1363-1370. |
| 92. | Pedotti R., Mitchell D., Wedemeyer J., Karpuj M., Chabas D., Hattab E.M., Tsai M., Galli S.J., Steinman L. An unexpected version of horror autotoxicus: anaphylactic shock to a self-peptide. Nat. Immunol. 2001;2:216-222. |
| 93. | Zhang L., Guo Y., Xia C.Q. Infusion of sulfosuccinimidyl-4-[N-maleimidomethyl]cyclohexane-1-carboxylate-conjugated MOG35-55-coupled spleen cells effectively prevents and reverses experimental autoimmune encephalomyelitis in mice. J. Immunol. Res. 2015;2015:129682. |
| 94. | Tse H.Y., Li J., Zhao X., Chen F., Ho P.P., Shaw M.K. Lessons learned from studies of natural resistance in murine experimental autoimmune encephalomyelitis. Curr. Trends Immunol. 2012;13:1-12. |
| 95. | Raine C.S., Traugott U., Stone S.H. Applications of chronic relapsing experimental allergic encephalomyelitis to the study of multiple sclerosis. In: Bauer H.J., Poser S., Ritter G., editors. Progress in Multiple Sclerosis Research. Springer Berlin Heidelberg; 1980. pp. 3-10. |
| 96. | Marcus J.F., Waubant E.L. Updates on clinically isolated syndrome and diagnostic criteria for multiple sclerosis. Neurohospitalist. 2013;3:65-80. |
| 97. | Berard J.L., Wolak K., Fournier S., David S. Characterization of relapsing-remitting and chronic forms of experimental autoimmune encephalomyelitis in C57BL/6 mice. Glia. 2010;58:434-445. |
| 98. | Bluestone J.A., Bour-Jordan H., Cheng M., Anderson M. T cells in the control of organ-specific autoimmunity. J. Clin. Invest. 2015;125:2250-2260. |
| 99. | Nie H., Zheng Y., Li R., Guo T.B., He D., Fang L., Liu X., Xiao L., Chen X., Wan B. Phosphorylation of FOXP3 controls regulatory T cell function and is inhibited by TNF-α in rheumatoid arthritis. Nat. Med. 2013;19:322-328. |
| 100. | Biswas M., Sarkar D., Kumar S.R., Nayak S., Rogers G.L., Markusic D.M., Liao G., Terhorst C., Herzog R.W. Synergy between rapamycin and FLT3 ligand enhances plasmacytoid dendritic cell-dependent induction of CD4+CD25+FoxP3+ Treg. Blood. 2015;125:2937-2947. |
| 101. | Battaglia M., Stabilini A., Roncarolo M.G. Rapamycin selectively expands CD4+CD25+FoxP3₊ regulatory T cells. Blood. 2005;105:4743-4748. |
| 102. | Liu T., Soong L., Liu G., König R., Chopra A.K. CD44 expression positively correlates with Foxp3 expression and suppressive function of CD4+ Treg cells. Biol. Direct. 2009;4:40. |
| 103. | Li M.O., Rudensky A.Y. T cell receptor signalling in the control of regulatory T cell differentiation and function. Nat. Rev. Immunol. 2016;16:220-233. |
| 104. | Lallana E.C., Fadul C.E. Toxicities of immunosuppressive treatment of autoimmune neurologic diseases. Curr. Neuropharmacol. 2011;9:468-477. |
| 105. | Gregori S., Passerini L., Roncarolo M.G. Clinical outlook for type-1 and FOXP3(+) T regulatory cell-based therapy. Front. Immunol. 2015;6:593. |
| 106. | Brunstein C.G., Blazar B.R., Miller J.S., Cao Q., Hippen K.L., McKenna D.H., Curtsinger J., McGlave P.B., Wagner J.E. Adoptive transfer of umbilical cord blood-derived regulatory T cells and early viral reactivation. Biol. Blood Marrow Transplant. 2013;19:1271-1273. |
| 107. | Josefowicz S.Z., Lu L.F., Rudensky A.Y. Regulatory T cells: mechanisms of differentiation and function. Annu. Rev. Immunol. 2012;30:531-564. |
| 108. | Desreumaux P., Foussat A., Allez M., Beaugerie L., Hébuterne X., Bouhnik Y., Nachury M., Brun V., Bastian H., Belmonte N. Safety and efficacy of antigen-specific regulatory T-cell therapy for patients with refractory Crohn’s disease. Gastroenterology. 2012;143:1207-1217. e1-e2. |
| 109. | Hoffman B.E., Ertl H.C., Terhorst C., High K.A., Herzog R.W. Gene therapy research at the frontiers of viral immunology. Front. Microbiol. 2012;3:182. |
| 110. | Cao O., Hoffman B.E., Moghimi B., Nayak S., Cooper M., Zhou S., Ertl H.C., High K.A., Herzog R.W. Impact of the underlying mutation and the route of vector administration on immune responses to factor IX in gene therapy for hemophilia B. Mol. Ther. 2009;17:1733-1742. |
| 111. | Chapman N.M., Chi H. mTOR signaling, Tregs and immune modulation. Immunotherapy. 2014;6:1295-1311. |
| 112. | Battaglia M., Stabilini A., Migliavacca B., Horejs-Hoeck J., Kaupper T., Roncarolo M.G. Rapamycin promotes expansion of functional CD4+CD25+FOXP3+ regulatory T cells of both healthy subjects and type 1 diabetic patients. J. Immunol. 2006;177:8338-8347. |

It should be understood that the examples and embodiments described herein are for illustrative purposes only and that various modifications or changes in light thereof may be suggested to persons skilled in the art and are to be included within the spirit and purview of this application and the scope of the appended claims. All references, including publications, patent applications and patents, cited herein are hereby incorporated by reference to the same extent as if each reference was individually and specifically indicated to be incorporated by reference and was set forth in its entirety herein. Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the present disclosure using terms such as “comprising”, “having”, “including” or “containing” with reference to an element or elements is intended to provide support for a similar aspect or embodiment of the present disclosure that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein can be made and executed without undue experimentation in light of the present disclosure. While the compositions and methods of this invention have been described in terms of exemplary embodiments, it will be apparent to those of skill in the art that variations may be applied to the compositions and methods and in the steps or in the sequence of steps of the method described herein without departing from the concept, spirit and scope of the present disclosure. More specifically, it will be apparent that certain agents that are chemically and/or physiologically related may be substituted for the agents described herein while the same or similar results would be achieved. All such similar substitutes and modifications apparent to those skilled in the art are deemed to be within the spirit, scope and concept of the present disclosure as defined by the appended claims.

What is claimed is:

1. A method of treating a mammal in need thereof comprising administering to the mammal a therapeutically-effective amount of:
(a) a first composition comprising a recombinant adeno-associated viral (rAAV) vector comprising a polynucleotide that comprises a first nucleic acid segment that encodes a mammalian myelin basic protein (MBP), a proteolipid protein (PLP), or a myelin oligodendrocyte glycoprotein (MOG) operably linked to a promoter that is capable of expressing the first nucleic acid segment in one or more cells of a mammalian liver; and
(b) a second composition comprising an agent selected from an mTOR inhibitor, a sphingosine analog, a glucocorticoid, and a monoclonal antibody.

2. The method of claim 1, wherein the first nucleic acid segment of the first composition encodes a first therapeutic molecule that comprises one of a myelin basic protein (MBP), a myelin oligodendrocyte glycoprotein (MOG), and a proteolipid protein (PLP).

3. The method of claim 1 or 2, wherein the first nucleic acid segment encodes a myelin basic protein (MBP), a proteolipid protein (PLP), or a myelin oligodendrocyte glycoprotein (MOG) that comprises an amino acid sequence that is at least 90% identical to the sequence as set forth in SEQ ID NO:1, SEQ ID NO:2, or SEQ ID NO:3.

4. The method of any one of claims 1-3, wherein the first nucleic acid segment encodes a myelin basic protein (MBP), a proteolipid protein (PLP), or a myelin oligodendrocyte glycoprotein (MOG) that comprises an amino acid sequence as set forth in SEQ ID NO:1, SEQ ID NO:2, or SEQ ID NO:3.

5. The method of claim 1 or 2, wherein the first nucleic acid segment encodes a myelin basic protein (MBP), proteolipid protein (PLP), or myelin oligodendrocyte glycoprotein (MOG) that comprises an amino acid sequence that is at least 90% identical to the sequence as set forth in SEQ ID NO:17, SEQ ID NO:11, or SEQ ID NO:15.

6. The method of claim 2 or 5, wherein the first nucleic acid segment encodes a myelin basic protein (MBP), proteolipid protein (PLP), or myelin oligodendrocyte glycoprotein (MOG) that comprises an amino acid sequence as set forth in SEQ ID NO:17, SEQ ID NO:11, or SEQ ID NO:15.

7. The method of any one of claims 1-6, wherein the MBP, PLP or MOG are of human origin.

8. The method of any one of claims 1-7, wherein the promoter is a hepatocyte-specific promoter.

9. The method of claim 8, wherein the hepatocyte-specific promoter comprises an albumin promoter, a human α₁-antitrypsin promoter, a transthyretin (TTR) promoter, a hepatic combinatorial bundle (HCB) promoter, or an apolipoprotein E (apoE) promoter.

10. The method of claim 8 or 9, wherein the hepatocyte-specific promoter comprises a hepatic combinatorial bundle (HCB) promoter.

11. The method of claim 8 or 9, wherein the hepatocyte-specific promoter comprises a human apolipoprotein E (hapoE) promoter.

12. The method of any one of claims 1-11, wherein the polynucleotide further comprises an enhancer, a post-transcriptional regulatory sequence, a polyadenylation signal, or any combination thereof, operably linked to the first nucleic acid segment.

13. The method of any one of claims 1-12, wherein the polynucleotide comprises AAV2 inverted terminal repeat sequences (ITRs).

14. The method of any one of claims 1-13, wherein the polynucleotide comprises a second nucleic acid segment encoding a second therapeutic molecule.

15. The method of claim 14, wherein:
(a) the second therapeutic molecule is MBP or PLP if the first therapeutic molecule is MOG;
(b) the second therapeutic molecule is MBP or MOG if the first therapeutic molecule is PLP; or
(c) the second therapeutic molecule is PLP or MOG if the first therapeutic molecule is MBP.

16. The method of claim 14 or 15, wherein the polynucleotide comprises a third nucleic acid segment encoding a third therapeutic molecule.

17. The method of claim 16, wherein
(a) the third therapeutic molecule is MOG, if the first and second therapeutic molecules comprise MBP and PLP;
(b) the third therapeutic molecule is PLP, if the first and second therapeutic molecules comprises MBP and MOG; or
(c) the third therapeutic molecule is MBP, if the first and second therapeutic molecule comprises MOG and PLP.

18. The method of any one of claims 1-17, wherein the polynucleotide encodes MOG, MBP, and PLP.

19. The method of any one of claims 15-18, wherein the second therapeutic molecule and/or the third therapeutic molecule comprises an amino acid sequence as set forth in SEQ ID NO:1, SEQ ID NO:2, or SEQ ID NO:3.

20. The method of any one of claims 1-17, wherein the MOG, the PLP, and/or the MBP comprises a full-length polypeptide.

21. The method of claim 14, wherein the second nucleic acid segment encodes a polypeptide, a peptide, a ribozyme, a peptide nucleic acid, an siRNA, an RNAi, an antisense oligonucleotide, an antisense polynucleotide, an antibody, an antigen binding fragment, or any combination thereof.

22. The method of claim 21, wherein the second nucleic acid segment encodes a proteolipid protein, a myelin oligodendrocyte, a glycoprotein, a myelin-associated glycoprotein, insulin, an islet-specific glucose-6-phosphatase catalytic subunit-related protein, a Preproinsulin, a glutamic decarboxylase, a tyrosine phosphatase like autoantigen, an insulinoma antigen- 2, an Islet cell antigen, a thyroid stimulating hormone (TSH) receptor, a thyrotropin receptor, an Aggrecan, a CD4+T cell epitope, a porin, or an acetylcholine receptor.

23. The method of any one of claims 1-22, wherein the rAAV vector is used to treat an autoimmune disease.

24. The method of claim 23, wherein the autoimmune disease is selected from multiple sclerosis, disseminated sclerosis, encephalomyelitis disseminata, optic neuritis, celiac disease, or an allergic disease.

25. The method of claim 23, wherein the autoimmune disease is multiple sclerosis.

26. The method of any one of claims 1-25, wherein the rAAV vector is of serotype AAV1, AAV3, AAV4, AAV5, AAV6, AAV7, AAV8, AAV9, AAV10, AAV2-AAV3 hybrid, AAVrh.10, AAVrh.74, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShHIO, AAV2(Y➔F), AAV8(Y733F), AAV2.15, AAV2.4, AAVM41, or AAVr3.45; or a derivative thereof.

27. The method of any one of claims 1-25, wherein the rAAV vector is of serotype AAV8.

28. The method of claim 26 or 27 wherein the rAAV vector is pseudotyped.

29. The method of any one of claims 1-28, wherein the agent of the second composition is an mTOR inhibitor.

30. The method of any one of claims 1-29, wherein the agent is rapamycin.

31. The method of claim 29 or 30, wherein the mTOR inhibitor is administered in a dose of 0.5 mg, 1 mg, 1.25 mg, 1.5 mg, 1.75 mg, 2 mg, 2.25 mg, 2.5 mg, 2.75 mg, 3 mg, 4 mg, 5 mg, or 6 mg per day.

32. The method of any one of claims 29-31, wherein the mTOR inhibitor is administered in a dose of 0.1 mg per day.

33. The method of any one of claims 1-28, wherein the agent of the second composition is a sphingosine analog.

34. The method of claim 33, wherein the agent is fingolimod.

35. The method of claim 33 or 34, wherein the sphingosine analog is administered in a dose of 0.025 mg or 0.05 mg per day.

36. The method of any one of claims 1-28, wherein the agent of the second composition is a monoclonal antibody.

37. The method of claim 36, wherein the agent is natalizumab, alemtuzumab, or ocrelizumab.

38. The method of claim 36 or 37, wherein the monoclonal antibody is administered in a dose of 300 mg every 28 days.

39. The method of any one of claims 1-28, wherein the agent of the second composition is a glucocorticoid.

40. The method of claim 39, wherein the agent is prednisolone.

41. The method of claim 39 or 40, wherein the agent is administered in a dose of 5 mg, 10 mg, 15 mg, 20 mg, 25 mg, 30 mg, 35 mg, 40 mg, 45 mg, 50 mg, 55 mg, or 60 mg daily.

42. The method of any one of claims 1-41, wherein the first composition and/or the second composition further comprises one or more pharmaceutically acceptable excipients.

43. The method of any one of claims 1-42, wherein the first composition is administered before the second composition, the first composition is administered after the second composition, or the first composition and the second composition are administered simultaneously.

44. The method of claim 43, wherein the first composition and the second composition are administered simultaneously.

45. The method of any one of claims 1-44, wherein the first composition is administered by intravenous injection.

46. The method of any one of claims 1-45, wherein the second composition is administered orally.

47. The method of any one of claims 1-45, wherein the second composition is administered by intravenous injection.

48. A method for preventing an autoimmune disease or inhibiting progression of the disease in a mammal, the method comprising systemically administering to the mammal the first composition and the second composition in accordance with claims 1-44 in an amount and for a time sufficient to prevent or inhibit progression of the autoimmune disease in the mammal.

49. The method of claim 49, wherein the mammal has, is suspected of having, is at risk for developing, or has been diagnosed with the autoimmune disease.

50. The method of claim 49 or 50, wherein the mammal is a newborn, an infant, a juvenile, an adult, or a young adult.

51. The method of any one of claims 48-50, wherein the mammal is a human.

52. The method of any one of claims 48-51, whereby expression of the therapeutic molecule in the mammal reduces CNS inflammation, inhibits demyelination, re-establishes immune tolerance to one or more neuroproteins, stimulates the production of endogenous antigen-specific regulatory T cells, or any combination thereof.

53. The method of any one of claims 48-52, wherein the autoimmune disease is multiple sclerosis.

54. The method of any one of claims 48-53, whereby progression of the autoimmune disease in the mammal is inhibited or reversed for at least 40 days, at least 50 days, at least 75 days, at least 100 days, at least 125 days, at least 150 days, or more than 150 days after administration of the first composition.

55. The method of any one of claims 48-54, whereby progression of the autoimmune disease in the mammal is inhibited or reversed for at least 50 days after administration of the first composition.

56. The method of any one of claims 48-55, wherein the first composition is administered to the mammal in a single injection.

57. The method of any one of claims 48-56, whereby expression of the therapeutic molecule in the mammal re-establishes immune tolerance to at least two different neuroprotein epitopes.

58. The method of claim 57, wherein the at least two different neuroprotein epitopes comprise different epitopes of a single neuroprotein.

59. The method of claim 58, wherein the single neuroprotein is a MOG protein.

60. The method of claim 57, wherein the at least two different neuroproteins comprise at least one epitope of a MOG protein and at least one epitope of a PLP protein.

61. The method of any one of claims 1-60, wherein the first nucleic acid segment encodes a full-length human MOG operably linked to a hepatocyte-specific promoter, further wherein the rAAV vector is of serotype AAV8.

62. Use of the first composition and the second composition in accordance with claims 1-61 as a medicament.
